(19) United States
(12) Patent Application Publication
(10) Pub. No.: US 2024/0197753 A1
(43) Pub. Date: Jun. 20, 2024

(54) PHARMACEUTICAL COMPOSITION FOR IMPROVING CARDIAC FUNCTION

(71) Applicants: OSAKA UNIVERSITY, Osaka (JP); CUORIPS INC., Tokyo (JP)

(72) Inventors: Yoshiki SAWA, Osaka (JP); Shigeru MIYAGAWA, Osaka (JP); Yoshiki SAKAI, Osaka (JP); Yasuhiro YANAGI, Hyogo (JP)

(73) Assignees: OSAKA UNIVERSITY, Osaka (JP); CUORIPS INC., Tokyo (JP)

(21) Appl. No.: 18/283,585

(22) PCT Filed: Mar. 25, 2022
(86) PCT No.: PCT/JP2022/014632

§ 371 Date: Sep. 22, 2023

(30) Foreign Application Priority Data
Mar. 25, 2021 (JP) 2021-052484

(51) Int. Cl. A61K 31/559; A61K 9/06; A61K 9/16; A61K 9/70; A61K 47/42; A61P 9/04

(52) U.S. Cl. CPC A61K 31/559; A61K 9/06; A61K 9/1647; A61K 9/7007; A61K 47/42; A61P 9/04

(57) ABSTRACT

An object is to provide a pharmaceutical composition that improves cardiac function when administered during coronary artery bypass surgery for ischemic cardiomyopathy. A pharmaceutical composition for improving cardiac function comprising: (A) a release formulation comprising at least poly(lactic-co-glycolic acid) (PLGA) and a prostaglandin 12 receptor agonist, the PLGA having an average molecular weight of 10000 to 30000; and (B) a release formulation comprising at least poly(lactic-co-glycolic acid) (PLGA) and a prostaglandin 12 receptor agonist, the PLGA having an average molecular weight of 40000 to 60000.

### TECHNICAL FIELD

The present disclosure relates to a pharmaceutical composition for improving cardiac function.

### BACKGROUND ART

ONO-1301, which is the active ingredient of the pharmaceutical composition of the present disclosure, is a low-molecular-weight synthetic compound with a non-prostaglandin skeleton, and is a selective prostaglandin 12 receptor (IP receptor) agonist with thromboxane A2 (TXA2) synthase inhibitory activity. Initially, it was examined as an oral platelet aggregation inhibitor; however, its development was suspended because the results of its efficacy (platelet aggregation inhibition) and side effects (epigastric pain, fever, cold sweat, diarrhea, etc.) in Phase I clinical trials indicated that its safety margin was narrow.

ONO-1301 acts on vascular smooth muscle cells, platelets, vascular endothelial cells, and the like to inhibit platelet aggregation and dilate blood vessels; however, subsequent studies have shown that ONO-1301, at concentrations equal to or less than 1/20 of its platelet aggregation inhibitory effect, acts as an in vivo regenerative factor inducer (regeneration inducer) that newly acts on IP receptors on fibroblasts, smooth muscle cells, etc. to increase cyclic adenosine monophosphate (cAMP) and induce the production of various regenerative factors (hepatocyte growth factor (HGF), vascular endothelial growth factor (VEGF), stromal cell-derived factor (SDF-1), high-mobility group box 1 (HMGB1), etc.). These effects have led to the discovery that ONO-1301 exhibits angiogenic, anti-apoptotic, anti-fibrosis, anti-inflammatory, and bone marrow mesenchymal stem cell (MSC) mobilization and accumulation effects (drug repositioning). Furthermore, due to its TXA2 synthase inhibitory effect, ONO-1301 suppresses resistance to IP receptors even after long-term administration, and also promotes the production of endogenous prostaglandin E2 (PGE2) and prostaglandin 12 (PGI2).

### SUMMARY OF INVENTION

### Technical Problem

An object of the present disclosure is to provide a pharmaceutical composition that improves cardiac function when administered during coronary artery bypass surgery for ischemic cardiomyopathy.

More specifically, an object of the present invention is to provide a pharmaceutical composition that can retain the blood concentration of ONO-1301, which is the active ingredient, within a certain concentration range for a certain period of time, that can exhibit a cardiac function improvement effect, and that has confirmed clinical safety and tolerability, by producing a sustained-release formulation of microspheres (MS), such as compound (A), which is a PGI2 receptor agonist.

### Solution to Problem

As a result of various considerations regarding the production of MS sustained-release formulations containing a PGI2 receptor agonist, the present inventors found that by mixing PGI2 receptor agonist-containing release formulations each containing poly(lactic-co-glycolic acid) (PLGA) with a different average molecular weight, the PGI2 receptor agonist is released within a certain concentration range for a certain period of time.

The present inventors found that the approved drugs YS-1402-Gelfoam and YS-1402-Beriplast are optimal for the attachment of the pharmaceutical composition of the present invention to the heart during coronary artery bypass surgery for ischemic cardiomyopathy.

The present invention has been completed through further trial and error based on these findings, and includes the following invention.

Item 1.

A pharmaceutical composition for improving cardiac function, comprising:
(A) a release formulation comprising at least poly(lactic-co-glycolic acid) (PLGA) and a prostaglandin 12 receptor agonist, the PLGA having an average molecular weight of 1000 to 30000; and
(B) a release formulation comprising at least poly(lactic-co-glycolic acid) (PLGA) and a prostaglandin I2 receptor agonist, the PLGA having an average molecular weight of 40000 to 60000.

Item 2.

The pharmaceutical composition for improving cardiac function according to Item 1, wherein the ratio of the release formulation (B) to the release formulation (A) (A:B) is 1:1 to 100:1 or 1:1 to 1:100.

Item 3.

The pharmaceutical composition for improving cardiac function according to Item 1 or 2, wherein the release formulation (A) comprises 0.5 to 50 mg of PGI2 receptor agonist in one vial, and/or the release formulation (B) comprises 0.5 to 50 mg of PGI2 receptor agonist in one vial.

Item 4.

The pharmaceutical composition according to any one of Items 1 to 3, comprising a patch liquid.

Item 5.

The pharmaceutical composition according to Item 4, wherein the patch liquid is a 5 w/v % mannitol aqueous solution comprising 0.2 w/v % of polysorbate.

Item 6.

The pharmaceutical composition according to any one of Items 1 to 5, comprising a gelatin patch.

Item 7.

The pharmaceutical composition according to Item 6, wherein the gelatin patch is a porous sterile formulation comprising 10 g of gelatin per 1000 cm³.

Item 8.

The pharmaceutical composition according to any one of Items 1 to 7, comprising a plasma fraction formulation.

Item 9.

The pharmaceutical composition according to Item 8, wherein the plasma fraction formulation comprises a fibrinogen powder, an aprotinin solution, a thrombin powder, and a calcium chloride solution.

Item 10.

The pharmaceutical composition according to any one of Items 1 to 9, comprising, as the prostaglandin I2 receptor agonist, at least a compound of the following formula (I) or a salt thereof:

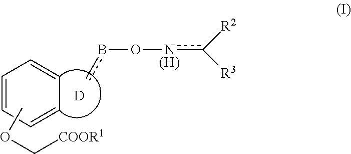

wherein

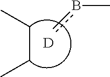

is

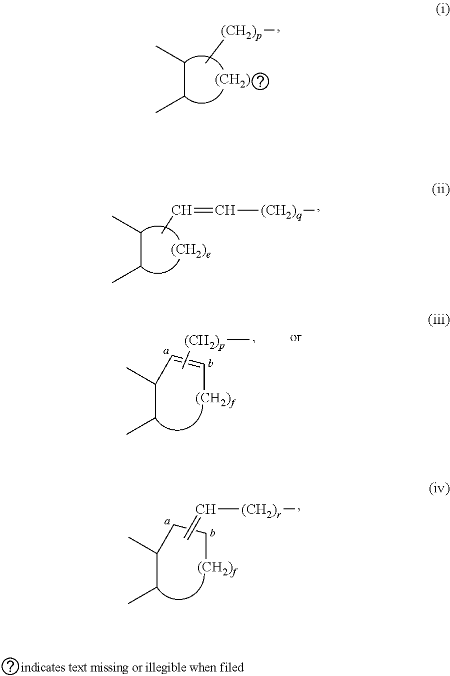

wherein
R¹ represents a hydrogen atom or a C1−4 alkyl group,
R² represents (i) a hydrogen atom, (ii) a C1−8 alkyl group, (iii) a phenyl group or a C4−7 cycloalkyl group, (iv) a 4- to 7-membered monocyclic ring containing one nitrogen atom, (v) a C1−4 alkyl group substituted with a benzene ring or a C4−7 cycloalkyl group, or (vi) a C1−4 alkyl group substituted with a 4- to 7-membered monocyclic ring containing one nitrogen atom,
R³ represents (i) a C1-8 alkyl group, (ii) a phenyl group or a C4−7 cycloalkyl group, (iii) a 4- to 7-membered monocyclic ring containing one nitrogen atom, (iv) a C1−4 alkyl group substituted with a benzene ring or a C4−7 cycloalkyl group, or (v) a C1−4 alkyl group substituted with a 4- to 7-membered monocyclic ring containing one nitrogen atom,
e represents an integer of 3 to 5,
f represents an integer of 1 to 3,
p represents an integer of 1 to 4,
q represents 1 or 2, and
r represents an integer of 1 to 3;
   provided that when

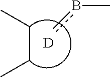

is a group represented by (iii) or (iv) above,
(CH₂)p- and ═CH—(CH₂)s- bind to the position of a or b on the ring, and
ring structures in R² and R³ are optionally substituted with 1 to 3 C1−4 alkyl groups, C1−4 alkoxy groups, halogen atoms, nitro groups, or trihalomethyl groups.

Item 11.

The pharmaceutical composition according to any one of Items 1 to 10, comprising, as the prostaglandin 12 receptor agonist, at least the following compound (A) or a salt thereof:
(A) ({5-[2-({[(1E)-phenyl(pyridin-3-yl)methylene]amino}oxy)ethyl]-7,8-dihydronaphthalen-1-yl}oxy)acetic acid (ONO-1301) represented by the following formula (II):

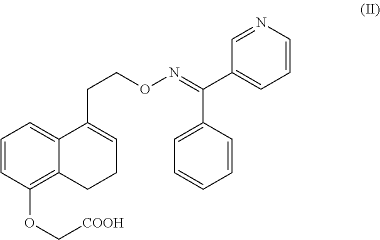 [SMILES: O=C(O)COc1cccc2c1CCC=C2CCO/N=C(\c1ccccc1)c1cccnc1]

Item 12.

The pharmaceutical composition according to any one of Items 1 to 11, wherein the pharmaceutical composition is a sheet patch.

Item 13.

The pharmaceutical composition according to any one of Items 1 to 12, wherein the pharmaceutical composition is administered to a patient with ischemic cardiomyopathy who undergoes coronary artery bypass surgery.

Item 14.

The pharmaceutical composition according to any one of Items 1 to 13, wherein the prostaglandin I2 receptor agonist is released over 4 weeks after administration.

Item 15.

The pharmaceutical composition according to any one of Items 1 to 14, which is a sustained-release formulation of microspheres (MS).

Item 16.

The pharmaceutical composition according to Item 15, wherein the sustained-release formulation has an average particle size of 3 to 300 μm.

### Advantageous Effects of Invention

The pharmaceutical composition of the present disclosure is useful to improve cardiac function because the blood concentration of ONO-1301, which is the active ingredient, remains within a certain concentration range for a certain period of time.

### BRIEF DESCRIPTION OF DRAWINGS

FIG. 1 is a schematic view of the preparation of the YS-1402 dosing sheet of the present invention for 30 mg dosing.

FIG. 2 is a schematic view of an administration method of the YS-1402 dosing sheet by heart attachment.

FIG. 3 is a view showing the breakdown of subjects of this trial.

FIG. 4 is a chart of time-dependent transition of blood ONO-1301 concentration when YS-1402−10 mg, 30 mg, or 100 mg was attached once at the time of thoracotomy for coronary artery bypass surgery. [Horizontal axis (time: equally spaced at each blood collection point), vertical axis (blood drug concentration: real number)]

FIG. 5 is a chart of time-dependent transition of blood ONO-1301 concentration when YS-1402−10 mg, 30 mg, or 100 mg was attached once at the time of thoracotomy for coronary artery bypass surgery. [Horizontal axis (time: proportional to the actual time), vertical axis (blood drug concentration: real number)]

FIG. 6 is a chart of time-dependent transition of blood ONO-1301 concentration when YS-1402−10 mg, 30 mg, or 100 mg was attached once at the time of thoracotomy for coronary artery bypass surgery. [Horizontal axis (time: proportional to the actual time), vertical axis (blood drug concentration: logarithm)]FIG. 7 is a chart of time-dependent transition of measured LVEF values in echocardiography when YS-1402−10 mg, 30 mg, or 100 mg was attached once at the time of thoracotomy for coronary artery bypass surgery.

FIG. 8 is a chart of time-dependent transition of the amount of change in LVEF in echocardiography when YS-1402−10 mg, 30 mg, or 100 mg was attached once at the time of thoracotomy for coronary artery bypass surgery.

FIG. 9 is a view showing the amount of change in LVEF from the baseline at 26 weeks after administration.

FIG. 10 is a view showing the correlation between the rate of change in total myocardial blood flow and the amount of change in LVEF from the baseline at 26 weeks after administration of the investigational drug.

FIG. 11 is a chart of time-dependent transition of measured CI values in cardiac-gated CT.

FIG. 12 is a chart of time-dependent transition of the rate of change in CI in cardiac-gated CT.

FIG. 13 is a view showing the rate of change in CI in cardiac-gated CT from the baseline at 26 weeks after administration.

FIG. 14 is a view showing the correlation between the rate of change in total myocardial blood flow and the rate of change in CI from the baseline at 26 weeks after administration of the investigational drug.

FIG. 15 is a chart of time-dependent transition of measured LVESVI values in cardiac-gated CT.

FIG. 16 is a chart of time-dependent transition of the rate of change in LVESVI in cardiac-gated CT.

FIG. 17 is a chart of time-dependent transition of measured LVEDVI values in cardiac-gated CT.

FIG. 18 is a chart of time-dependent transition of the rate of change in LVEDVI in cardiac-gated CT.

FIG. 19 is a chart of time-dependent transition of measured LVDs values in echocardiography.

FIG. 20 is a chart of time-dependent transition of the rate of change in LVDs in echocardiography.

FIG. 21 is a chart of time-dependent transition of measured LVDd values in echocardiography.

FIG. 22 is a chart of time-dependent transition of the rate of change in LVDd in echocardiography.

FIG. 23 is a chart of time-dependent transition of measured CTR values in chest X-ray.

FIG. 24 is a chart of time-dependent transition of the amount of change in CTR in chest X-ray.

FIG. 25 shows charts of time-dependent transition of the NYHA classification. Percentages were calculated using a population of six cases. The basic number of subjects to be evaluated was six; however, the number of subjects was five at 26 weeks after administration in the placebo group and the YS-1402 100 mg group because one case in each group discontinued during the course of treatment, and one case in the 30 mg group had right hemiparesis and could not be measured. Thus, there were five cases at all measurement points.

FIG. 26 is a chart of time-dependent transition of measured values of 6-minute walking distance.

FIG. 27 is a chart of time-dependent transition of the rate of change in 6-minute walking distance.

FIG. 28 is a view showing the rate of change in 6-minute walking distance from the baseline at 26 weeks after administration.

FIG. 29 is a view showing the rate of change in 6-minute walking distance from the baseline when excluding a case of congestive heart failure due to poor compliance in the YS-1402 30 mg group.

FIG. 30A is a chart of time-dependent transition of measured values of RCA resting myocardial blood flow by ammonia PET.

FIG. 30B is a chart of time-dependent transition of measured values of LAD resting myocardial blood flow by ammonia PET.

FIG. 30C is a chart of time-dependent transition of measured values of LCX resting myocardial blood flow by ammonia PET.

FIG. 30D is a chart of time-dependent transition of measured values of total myocardial blood flow by ammonia PET.

FIG. 31A is a chart of time-dependent transition of the rate of change in RCA resting myocardial blood flow from the baseline by ammonia PET.

FIG. 31B is a chart of time-dependent transition of the rate of change in LAD resting myocardial blood flow from the baseline by ammonia PET.

FIG. 31C is a chart of time-dependent transition of the rate of change in LCX resting myocardial blood flow from the baseline by ammonia PET.

FIG. 31D is a chart of time-dependent transition of the rate of change in total myocardial blood flow from the baseline by ammonia PET.

FIG. 32 is a view showing the rate of change in total myocardial blood flow from the baseline at 26 weeks after administration.

FIG. 33 is a view showing the rate of change in LAD resting myocardial blood flow from the baseline at 26 weeks after administration.

FIG. 34 is a view showing the correlation between the blood concentration (AUC0-t) and the rate of change in total myocardial blood flow from the baseline at 26 weeks after administration of the investigational drug.

FIG. 35 is a view showing the correlation between the blood concentration (Cmax) and the rate of change in total myocardial blood flow from the baseline at 26 weeks after administration of the investigational drug.

FIG. 36 is a view showing the correlation between AUC0-t and the rate of change in LAD resting myocardial blood flow from the baseline at 26 weeks after administration of the investigational drug.

FIG. 37 is a chart of time-dependent transition of measured blood BNP concentration values.

FIG. 38 is a chart of time-dependent transition of the rate of change in blood BNP concentration.

FIG. 39A is a chart of time-dependent transition of measured score values of the SF-36 subscale [physical functioning] set for QOL assessment.

FIG. 39B is a chart of time-dependent transition of measured score values of the SF-36 subscale [role physical] set for QOL assessment.

FIG. 39C is a chart of time-dependent transition of measured score values of the SF-36 subscale [bodily pain] set for QOL assessment.

FIG. 39D is a chart of time-dependent transition of measured score values of the SF-36 subscale [general health] set for QOL assessment.

FIG. 39E is a chart of time-dependent transition of measured score values of the SF-36 subscale [vitality] set for QOL assessment.

FIG. 39F is a chart of time-dependent transition of measured score values of the SF-36 subscale [social functioning] set for QOL assessment.

FIG. 39G is a chart of time-dependent transition of measured score values of the SF-36 subscale [role emotional] set for QOL assessment.

FIG. 39H is a chart of time-dependent transition of measured score values of the SF-36 subscale [mental health] set for QOL assessment.

FIG. 40A is a chart of time-dependent transition of the amount of change in the scores of the SF-36 subscale [physical functioning] set for QOL assessment.

FIG. 40B is a chart of time-dependent transition of the amount of change in the scores of the SF-36 subscale [role physical] set for QOL assessment.

FIG. 40C is a chart of time-dependent transition of the amount of change in the scores of the SF-36 subscale [bodily pain] set for QOL assessment.

FIG. 40D is a chart of time-dependent transition of the amount of change in the scores of the SF-36 subscale [general health] set for QOL assessment.

FIG. 40E is a chart of time-dependent transition of the amount of change in the scores of the SF-36 subscale [vitality] set for QOL assessment.

FIG. 40F is a chart of time-dependent transition of the amount of change in the scores of the SF-36 subscale [social functioning] set for QOL assessment.

FIG. 40G is a chart of time-dependent transition of the amount of change in the scores of the SF-36 subscale [role emotional] set for QOL assessment.

FIG. 40H is a chart of time-dependent transition of the amount of change in the scores of the SF-36 subscale [mental health] set for QOL assessment.

FIG. 41 show views showing the evaluation of the relationship (FAS) between echocardiography (LVEF) and cardiac-gated CT (LVESVI).

### DESCRIPTION OF EMBODIMENTS

1. Pharmaceutical composition for improving cardiac function

The pharmaceutical composition for improving cardiac function of the present invention comprises:
a release formulation (A) comprising poly(lactic-co-glycolic acid) (PLGA) having an average molecular weight of 10000 to 30000 and a prostaglandin I2 (PGI2) receptor agonist;
a release formulation (B) comprising poly(lactic-co-glycolic acid) (PLGA) having an average molecular weight of 40000 to 60000 and a prostaglandin 12 (PGI2) receptor agonist;
a patch liquid;
a gelatin patch; and
a plasma fraction formulation.

### 1.1. Release Formulation Comprising Poly(Lactic-Co-Glycolic Acid) (PLGA) and Prostaglandin 12 (PGI2) Receptor Agonist

The pharmaceutical composition for improving cardiac function of the present invention comprises release formulations (A) and (B) comprising two types of PLGA having different average molecular weights, and a PGI2 receptor agonist.

The pharmaceutical composition for improving cardiac function of the present invention is prepared (suspended) before use using two types of release formulations (A) and (B). The release formulation (A) is a sterile formulation produced by using PLGA having an average molecular weight of 10000 to 30000, and is a two-week release formulation containing 0.5 to 50 mg of PGI2 receptor agonist in one vial. The release formulation (B) is a sterile formulation produced by using PLGA having an average molecular weight of 40000 to 60000, and is a four-week release formulation containing 0.5 to 50 mg of PGI2 receptor agonist in one vial.

The “average molecular weight” may be any molecular weight, including weight average molecular weight and number average molecular weight.

In the pharmaceutical composition for improving cardiac function of the present invention, the content ratio of the two types of release formulations (A) and (B) (A:B) is not particularly limited, but is preferably 1:1 to 100:1 or 1:1 to 1:100, and more preferably 1:1.

The contents of PLGA and the PGI2 receptor agonist are not particularly limited; however, it is preferable to contain 1 to 100%, preferably 5 to 90%, of the PGI2 receptor agonist relative to PLGA1 in the total amount of the sustained-release formulation of the present invention.

The PGI2 receptor agonist used in the pharmaceutical composition for improving cardiac function of the present invention is not particularly limited, and known PGI2 receptor agonists can be preferably used. Examples of known PGI2 receptor agonists include pharmaceutical compositions, PGI2 derivatives, and PGE derivatives, which are compounds represented by formula (I) or salts thereof:

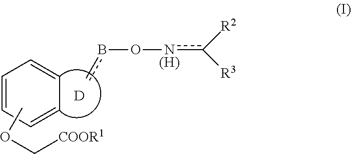

wherein

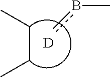

is

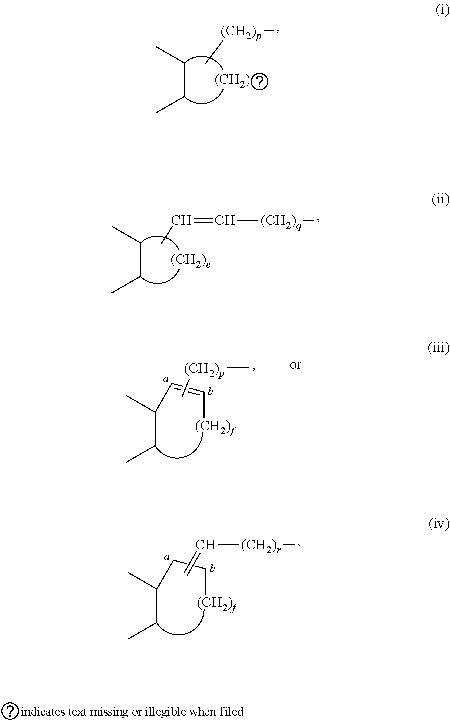

wherein
R¹ represents a hydrogen atom or a C1−4 alkyl group,
R² represents (i) a hydrogen atom, (ii) a C1−8 alkyl group, (iii) a phenyl group or a C4−7 cycloalkyl group, (iv) a 4- to 7-membered monocyclic ring containing one nitrogen atom, (v) a C1−4 alkyl group substituted with a benzene ring or a C4−7 cycloalkyl group, or (vi) a C1−4 alkyl group substituted with a 4- to 7-membered monocyclic ring containing one nitrogen atom,
R³ represents (i) a C1−8 alkyl group, (ii) a phenyl group or a C4−7 cycloalkyl group, (iii) a 4- to 7-membered monocyclic ring containing one nitrogen atom, (iv) a C1−4 alkyl group substituted with a benzene ring or a C4−7 cycloalkyl group, or (v) a C1−4 alkyl group substituted with a 4- to 7-membered monocyclic ring containing one nitrogen atom,
e represents an integer of 3 to 5,
f represents an integer of 1 to 3,
p represents an integer of 1 to 4,
q represents 1 or 2, and
r represents an integer of 1 to 3;
   provided that when

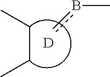

is a group represented by (iii) or (iv) above,
(CH₂)p- and ═CH—(CH₂)s- bind to the position of a or b on the ring, and
ring structures in R² and R³ are optionally substituted with 1 to 3 C1−4 alkyl groups, C1−4 alkoxy groups, halogen atoms, nitro groups, or trihalomethyl groups.

Preferably, the PGI2 receptor agonist is:
(A) ({5-[2-({[(1E)-phenyl(pyridin-3-yl)methylene]amino}oxy)ethyl]-7,8-dihydronaphthalen-1-yl}oxy)acetic acid represented by the following formula (II):

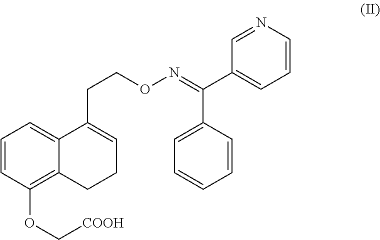 [SMILES: O=C(O)COc1cccc2c1CCC=C2CCO/N=C(\c1ccccc1)c1cccnc1]

(CAS 176391−41−6; compound (A) (ONO-1301));
(B) a carbacyclin-based PGI2 derivative, including (±)−(1R,2R,3aS,8bS)-2,3,3a,8b-tetrahydro-2-hydroxy-1-[(E)−(3S,4RS)-3-hydroxy-4-methyl-1-octen-6-ynyl]-1H-cyclopenta[b]benzofuran-5-butanoic acid sodium salt (CAS: 88475−69−8; beraprost) etc. (compound (B));
(C) [4-(5,6-diphenylpyrazinyl) (1-methylethyl)amino]butoxy]-acetic acid (CAS: 475085−57−5; MRE-269; compound (C));
(D) a PGE derivative, including (2E)-7{−(1R,2R,3R)-3-hydroxy-2[−(1E,3S,5S)-3-hydroxy-5-methylnon-1-en-1-yl]-5-oxocyclopentyl}-hept-2-enoic acid (CAS: 74397−12−9; limaprost), ornoprostyl; 17S,20-dimethyl-6-oxo-PGE1 methyl ester, enprostyl, misoprostol, etc. (compound (D)); or
(E) 2-{4-[(5,6-diphenylpyrazin-2-)yl) (propan-2-yl)amino]butoxy}-N-(methanesulfonyl)acetamide (CAS: 475086−01−2; selexipag; NS-304 (compound (E)).

### 1.2. Patch Liquid

The pharmaceutical composition for improving cardiac function of the present invention comprises a patch liquid for use in preparation before use of the two types of release formulations (A) and (B).

The patch liquid is preferably a 5 w/v % mannitol aqueous solution containing 0.2 w/v % of polysorbate.

### 1.3. Gelatin Patch

The pharmaceutical composition for improving cardiac function of the present invention comprises a gelatin patch for use in the production of a dosing sheet of the pharmaceutical composition for improving cardiac function of the present invention.

As the gelatin patch, it is preferable to use the commercial product YS-1402-Gelfoam (registered trademark, Pfizer Japan Inc.). YS-1402-Gelfoam is a spongy sheet agent obtained by blowing bubbles into Japanese Pharmacopoeia gelatin, and is a white porous sterile formulation containing 10 g of Japanese Pharmacopoeia gelatin per 1000 cm³. Although YS-1402-Gelfoam is an approved drug, the use thereof in the present invention is off-label use.

### 1.4. Plasma Fraction Formulation

The pharmaceutical composition for improving cardiac function of the present invention comprises a plasma fraction formulation as the physiological tissue adhesive of the present invention in order to prevent the dosing sheet of the pharmaceutical composition for improving cardiac function of the present invention applied to the heart from detaching from the heart.

As the plasma fraction formulation, it is preferable to use YS-1402-Beriplast ((registered trademark) P Combi-Set Tissue adhesion: a plasma fraction formulation, CSL Behring). YS-1402-Beriplast contains a fibrinogen powder, an aprotinin solution, a thrombin powder, and a calcium chloride solution. YS-1402-Beriplast is used by dropping around the attachment site of the dosing sheet of the sustained-release formulation of the present invention. Although YS-1402-Beriplast is an approved drug, the use thereof in the present invention is off-label use.

YS-1402-Beriplast is 1 set for 3 ml formulation, and there are vials 1 to 4. Vial 1 contains 240 mg of fibrinogen and human blood coagulation factor XIII (180 international units), vial 2 contains an aprotinin solution (3000 KIE), vial 3 contains Japanese Pharmacopoeia thrombin (900 units), and vial 4 contains Japanese Pharmacopoeia calcium chloride hydrate (17.64 mg). Vials 1 and 2 are mixed to form liquid A, and vials 3 and 4 are mixed to form liquid B. Liquids A and B are mixed to form a fibrin glue.

### 1.5. Other Components

The pharmaceutical composition for improving cardiac function of the present invention may contain components other those mentioned above as long as the effects of the present invention are not impaired.

2. Form, preparation method, and clinical administration method of pharmaceutical composition for improving cardiac function

The pharmaceutical composition for improving cardiac function of the present invention is a sustained-release formulation of microspheres (MS).

Although it is not particularly limited, the sustained-release formulation of the present invention preferably has an average particle size of about 3 to 300 μm, more preferably about 5 to 200, and even more preferably about 10 to 100. In the present specification, the “particle size” refers to the diameter of particles measured by any method including a laser diffraction method. The method for adjusting the particle size is not particularly limited.

The form of the pharmaceutical composition for improving cardiac function of the present invention is not particularly limited, but is preferably a sheet-like MS sustained-release formulation.

The method for preparing the dosing sheet of the pharmaceutical composition for improving cardiac function of the present invention comprises the following steps:
(a) suspending two types of release formulations (A) and (B) using a patch liquid; and
(b) dropping the suspension obtained in step (a) on YS-1402-Gelfoam to form a dosing sheet.

In step (a), hydrolysis starts when the patch liquid is added, and the release of a PGI2 receptor agonist, which is the active ingredient, starts. Therefore, administration by heart attachment is performed within, as a guideline, 6 hours after the start of the preparation of the dosing sheet. In order to prevent hydrolysis, the dosing sheet is kept refrigerated in a sterile petri dish.

The method for clinically administering the dosing sheet of the pharmaceutical composition for improving cardiac function of the present invention to the heart comprises the following steps:
(c) attaching the dosing sheet obtained in step (b) to a site of the cardiac ischemic area where graft running is not affected; and
(d) dropping YS-1402-Beriplast around the attachment area to enclose the dosing sheet obtained in step (b).

It is preferable that the pharmaceutical composition for improving cardiac function of the present invention is administered at the end of coronary artery bypass surgery, and that the PGI2 receptor agonist is released over 4 weeks after administration.

In the present disclosure, the phrase “released over 4 weeks” means that the blood concentration of the PGI2 receptor agonist, which is the active ingredient, remains within a certain concentration range over 4 weeks after administration of the pharmaceutical composition for improving cardiac function of the present invention.

The dose of the pharmaceutical composition for improving cardiac function of the present invention depends on the severity of the symptom to be treated, but is preferably an amount in which the dose of the PGI2 receptor agonist, which is the active ingredient contained therein, is 1000 mg or less.

### EXAMPLES

The present invention is described below with reference to Examples; however, the present invention is not limited to these Examples and the like.

### Preparation of YS-1402 Dosing Sheets and Placebo Formulation

Dosing sheets of the cardiac function-improving composition of the present invention (hereinafter referred to as “YS-1402”) with an ONO-1301 content of 10 mg, 30 mg, or 100 mg, and a placebo formulation not containing ONO-1301 were prepared (Groups 1 to 3).
Group 1: YS-1402 (ONO-1301 content: 10 mg) and placebo groups
Group 2: YS-1402 (ONO-1301 content: 30 mg) and placebo groups
Group 3: YS-1402 (ONO-1301 content: 100 mg) and placebo groups

The YS-1402 dosing sheets with an ONO-1301 content of 10 mg, 30 mg, or 100 mg, and the placebo formulation can be produced, for example, in the following manner. The formulations shown in Table 1 were used for the preparation.

TABLE 1

| Investigational | | |
|---|---|---|
| drug name | Content, dosage form | Production code/number |
| YS-1402 | A sustained-release formulation of microspheres (MS) containing about | 10 mg (active, placebo) |
| | 15% of ONO-1301 relative to two different molecular weights (20,000 | groups: J561P |
| | and 50,000) of poly(lactic-co-glycolic acid). This is prepared (suspended) | 30 mg (active, placebo) |
| | before use using YS-4102-1 and YS-4102-2. | groups: J562P |
| YS-1402-1 | A sterile formulation produced by using poly(lactic-co-glycolic acid) | 100 mg (active, placebo) |
| | having a molecular weight of about 20,000 and containing 5 mg of | groups: J791P |
| | ONO-1301 in one vial. YS-1402-1 is a two-week release formulation. | |
| YS-1402-2 | A sterile formulation produced by using poly(lactic-co-glycolic acid) | |
| | having a molecular weight of about 50,000 and containing 5 mg of | |
| | ONO-1301 in one vial. YS-1402-2 is a four-week release formulation. | |
| Patch liquid | A sterile formulation containing a solution for suspension before use of | J563P |
| | YS-1402-1 and YS-1402-2 (component: a 5 w/v % mannitol aqueous | J792P |
| | solution containing 0.2 w/v % of polysorbate) | J861P |
| YS-1402- | A white porous sterile formulation containing 10 g of Japanese | HE479 |
| Gelfoam | Pharmacopoeia gelatin per 1,000 cm³ | JU497 |
| | | KK507 |
| YS-1402- | Fibrinogen powder: white lump lyophilizer | 609205A |
| Beriplast | Aprotinin solution: colorless transparent liquid | 609226A |
| | Thrombin powder: white to pale yellow amorphous substance | 609230A |
| | Calcium chloride solution: colorless transparent liquid | 609291A |
| | | 609331A |
| | | 609346A |
| | | 609400A |
| | | 609437A |
| | | 609459A |
| | | P100042737 |

### Preparation of YS-1402 Dosing Sheet Containing 10 mg of ONO-1301

An patch liquid is collected using a syringe fitted with a 20 G or larger needle. The needle is removed from the syringe collecting the patch liquid, and a needle of the same size is attached. This is used to add the patch liquid to a vial of YS-1402−2. After stirring thoroughly and collecting the liquid inside the vial, the syringe is removed from the needle. The same liquid is placed in a vial of YS-1402−1, after stirring thoroughly, the liquid inside the vial is collected, and the syringe is removed from the needle. The syringe is connected to a three-way stopcock. A new syringe is taken out to collect the patch liquid via the needle inserted into the vial of the patch liquid, each vial is then washed in turn, and the second syringe is attached to the three-way stopcock to which the first syringe is connected. The liquids collected in the syringes are mixed without foaming via the three-way stopcock. The mixture is collected in one syringe and evenly added to two sheets of YS-1402-Gelfoam. After adding the suspension, YS-1402-Beriplast is dropped to fix and hide MS powder. After dropping, the sheets are attached to the heart as soon as possible.

### Preparation of YS-1402 dosing sheet containing 30 mg of ONO-1301

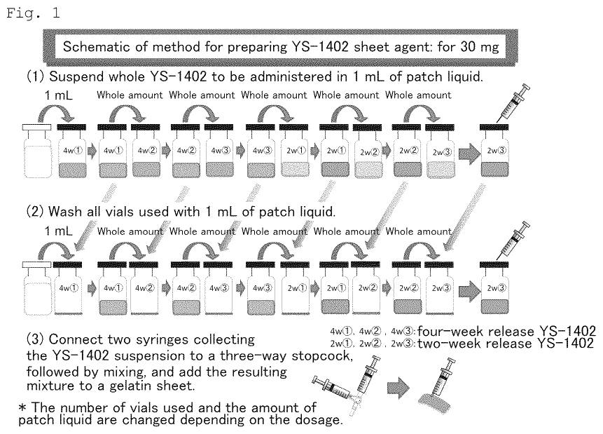

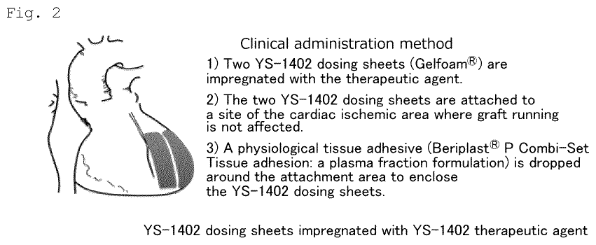

FIG. 1 shows a schematic view of the preparation of a YS-1402 dosing sheet containing 30 mg of ONO-1301. An patch liquid is collected using a syringe fitted with a 20 G or larger needle. The needle is removed from the syringe collecting the patch liquid, and a needle of the same size is attached. This is used to add the patch liquid to a vial of YS-1402−2. After stirring thoroughly and collecting the liquid inside the vial, the syringe is removed from the needle. A new needle is fitted thereto, and the same liquid is placed in a vial of YS-1402−2, after stirring thoroughly, the liquid inside the vial is collected, and the syringe is removed from the needle. This operation is performed for 3 vials of YS-1402−2 and 3 vials of YS-1402−1, and the syringe is connected to a three-way stopcock. A new syringe is taken out to collect the patch liquid via the needle inserted into the vial of the patch liquid, each vial is then washed in turn, and the second syringe is attached to the three-way stopcock to which the first syringe is connected. The liquids collected in the syringes are mixed without foaming via the three-way stopcock. The mixture is collected in one syringe and evenly added to two sheets of YS-1402-Gelfoam. After adding the suspension, a physiological tissue adhesive (YS-1402-Beriplast) is dropped to fix and hide MS powder. After dropping, the sheets are attached to the heart as soon as possible.

### Preparation of YS-1402 Dosing Sheet Containing 100 mg of ONO-1301

An patch liquid is collected using a syringe fitted with a 20 G or larger needle. The needle is removed from the syringe collecting the patch liquid, and a needle of the same size is attached. This is used to add the patch liquid to a vial of YS-1402−2. After stirring thoroughly and collecting the liquid inside the vial, the syringe is removed from the needle. A new needle is fitted thereto, and the same liquid is placed in a vial of YS-1402−2, after stirring thoroughly, the liquid inside the vial is collected, and the syringe is removed from the needle. This operation is performed for 10 vials of YS-1402−2 and 10 vials of YS-1402−1, and the syringe is connected to a three-way stopcock. A new syringe is taken out to collect the patch liquid via the needle inserted into the vial of the patch liquid, each vial is then washed in turn, and the second syringe is attached to the three-way stopcock to which the first syringe is connected. The liquids collected in the syringes are mixed without foaming via the three-way stopcock. The mixture is collected in one syringe and evenly added to two sheets of YS-1402-Gelfoam. After adding the suspension, a physiological tissue adhesive (YS-1402-Beriplast) is dropped to fix and hide MS powder. After dropping, the sheets are attached to the heart as soon as possible.

### Preparation of Placebo Formulation

After adding the patch liquid evenly to two similar sheets of YS-1402-Gelfoam, a physiological tissue adhesive (YS-1402-Beriplast) is dropped to hide the addition part. After dropping, the sheets are attached to the heart as soon as possible.

### Selection of Administration Site and Administration Method

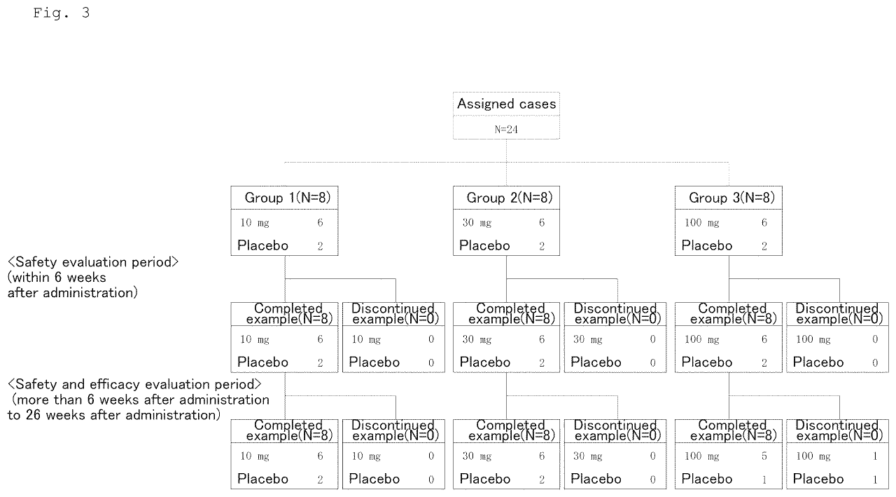

YS-1402 dosing sheets were attached to the left ventricle at the end of coronary artery bypass surgery through thoracotomy (FIG. 2).

For the site of attachment, the area of reduced myocardial blood flow was identified in advance by preoperative ammonia positron emission tomography (ammonia PET). Lesion sites such as highly fibrotic regions and poor contraction regions in complex lesions and multivessel lesions where complete blood flow recovery to cardiac ischemia was difficult were also identified visually and tactilely in the coronary artery bypass surgery, and two YS-1402 dosing sheets were attached to the site, including the peripheral part, where graft running was not affected.

### Dosing Frequency

Since the investigational drug was administered at the time of thoracotomy for coronary artery bypass surgery, the dosing frequency was once.

### Post-Dose Treatment

In order to prevent the two YS-1402 dosing sheets attached to the heart from detaching from the heart, YS-1402-Beriplast was dropped around the area where the YS-1402 dosing sheets were attached, to enclose the YS-1402 dosing sheets, and the chest was closed.

### Identification of Therapeutic Agents

YS-1402 is a sustained-release formulation of MS with a particle size of about 30 μm (average) containing about 15% of ONO-1301 relative to two different molecular weights (20000 and 50000) of poly(lactic-co-glycolic acid) (1:1). The formulation is designed so that the blood concentration of ONO-1301 remains within a certain concentration range over about 4 weeks after administration.

### Evaluation of Safety and Tolerability

The safety and tolerability were comprehensively evaluated for the following items 1) to 5).

### 1) Adverse Events

This item was set to evaluate safety and tolerability when YS-1402 was attached to the heart. The type, severity, seriousness, occurrence frequency, and occurrence period of adverse events were checked.

### 2) General Clinical Examination

This item was set to evaluate the overall safety when YS-1402 was attached to the heart (Table 2).
(1) Hematology test
(2) Blood biochemistry test
(3) Blood coagulation system test
3) Clinical symptoms

This item was set to evaluate the overall safety when YS-1402 was attached to the heart.
(1) Vital signs: blood pressure (diastolic, systolic), heart rate, respiratory rate, body temperature (armpit)
(2) Subjective symptoms: In particular, the presence or absence of diarrhea and heaviness of the head was checked.
(3) Physical findings: The presence or absence of wet rales, edema, and extra heart sounds was checked.
4) Resting standard 12-lead electrocardiography and Holter electrocardiography

This item was set to check the presence or absence of arrhythmia and myocardial ischemia. The presence or absence of arrhythmia, abnormal Q waves (12-lead electrocardiography only), and abnormal findings was checked.
5) Presence or absence and degree of bleeding after attachment (post-operation)

The presence or absence and degree of postoperative bleeding after heart attachment were evaluated according to the BARC definition for bleeding, which is often used to evaluate bleeding after coronary artery bypass surgery. The BARC definition for bleeding is shown in Table 3.

TABLE 2

| Test item | Measurement item | Basis for setting |
|---|---|---|
| Hematology test | White blood cells, red blood cells, | Indicators of blood disorders and anemia |
| | hemoglobin, hematocrit, platelets, | |
| | differential white blood cells (Neu, Ly, | |
| | Mo, Eo, Ba) | |
| Blood biochemistry | AST(GOT), ALT(GPT), LDH, ALP | Indicators of liver damage |
| test | BUN, creatinine | Indicators of renal damage |
| | Blood sugar | Indicator of abnormal glucose metabolism |
| | Electrolytes (Na, K, Cl) | Indicators of electrolyte abnormality |
| | CK, CK einzyme | Indicators of myocardial necrosis |
| | Uric acid | Indicator of abnormal nucleic acid metabolism |
| | TG, T-Cho, LDL-Cho, | Indicators of abnormal lipid metabolism |
| | Albumin | Indicator of nutritional status |
| | CRP | Indicator of inflammation |
| Blood coagulation | Prothrombin time (PT) | Indicators of bleeding tendency |
| system test | Activated partial thromboplastin time | |
| | (APTT) | |

TABLE 3

| Type | | Definition |
|---|---|---|
| 0 | | No bleeding |
| 1 | | Bleeding that is not actionable and does not cause the patient to seek unscheduled performance of |
| | | studies, hospitalization, or treatment by a healthcare professional; may include episodes leading to |
| | | self-discontinuation of medical therapy by the patient without consulting a healthcare professional |
| 2 | | Any overt, actionable sign of hemorrhage (e.g., more bleeding than would be expected for a clinical |
| | | circumstance, including bleeding found by imaging alone) that does not fit the criteria for type 3, 4, |
| | | or 5 but does meet at least one of the following criteria: |
| | | (1) requiring nonsurgical, medical intervention by a healthcare professional, |
| | | (2) leading to hospitalization or increased level of care, or |
| | | (3) prompting evaluation |
| 3 | 3a | Overt bleeding plus hemoglobin drop of 3 to <5 g/dL* (provided hemoglobin drop is related to |
| | | bleed) |
| | | Any transfusion with overt bleeding |
| | 3b | Overt bleeding plus hemoglobin drop ≥5 g/dL* (provided hemoglobin drop is related to bleed) |
| | | Cardiac tamponade |
| | | Bleeding requiring surgical intervention for control (excluding dental/nasal/skin/hemorrhoid) |
| | | Bleeding requiring intravenous vasoactive agents |
| | 3c | Intracranial hemorrhage (does not include microbleeds or hemorrhagic transformation, does |
| | | include intraspinal) |
| | | Subcategories confirmed by autopsy or imaging or lumbar puncture |
| | | Intraocular bleed compromising vision |
| 4^{*1} | | Perioperative intracranial bleeding within 48 h |
| | | Reoperation after closure of sternotomy for the purpose of controlling bleeding |
| | | Transfusion of ≥5 U whole blood or packed red blood cells within a 48-h period |
| | | Chest tube output ≥2 L within a 24-h period |
| 5^{*2} | 5a | Probable fatal bleeding; no autopsy or imaging confirmation but clinically suspicious |
| | 5b | Definite fatal bleeding; overt bleeding or autopsy or imaging confirmation |

*¹Bleeding associated with coronary artery bypass surgery,
*²fatal bleeding

### Evaluation of Pharmacokinetics

### 1) Secondary Endpoint

### (1) Plasma Drug Concentration

This was set to investigate the pharmacokinetics of active 0140−1301 in blood. It was evaluated by the pharmacokinetic parameter (Cmax) after attachment. Blood concentration measurement points were 1, 3, 6, and 24 hours, 7 days, 10 days, 14 days, 28 days (4 weeks), and 6 weeks after administration, and the measurement was also performed 8 weeks after administration to confirm the disappearance of blood concentrations. 5 ml of each blood sample was collected in a blood collection tube with sodium heparin, kept ice-cold until centrifugation, and centrifuged (3000 rpm×10 min) immediately. Plasma fractions were collected and then stored frozen (−20° C.).

### 2) Exploratory Endpoints

(1) Amount of change in left ventricular ejection fraction (LVEF) at 26 weeks after attachment

This was set to evaluate the contractility of the entire left ventricle. The improvement in the contractility of the entire left ventricle was evaluated by the amount of change in LVEF obtained by echocardiography (0, 26 weeks).

### (2) Changes in Left Ventricular Pump Function (Cardiac Index [CT]) Before and After Attachment

In order to evaluate the left heart pump function, CT measurement by CT scan was set. CT (ml/min/m2) was calculated by the following equations.

CT (ml/min/m2)=((LVEDV (ml))−(LVESV (ml)))×(heart rate (beats/min))/(body surface area (m2))

Body surface area (m2)=weight (kg) 0.425×height (cm) 0.725×0.007 184 (Dubois' formula)

For the height and weight, values measured at the following time points were used.
Height: a value measured at the time of screening
Weight: a value measured on the day of cardiac-gated CT scan
   (3) Changes in left ventricular remodeling before and after attachment

In order to multilaterally evaluate the inhibition of left ventricular remodeling, the following items were set.

### [1] LVESVI

LVESVI is an index of ventricular remodeling for indicating the progression of heart failure. LVESVI has been reported as a life prognostic factor in many documents. Changes in LVESVI are correlated with changes in prognosis, and the direction and magnitude of the changes in LVESVI are considered to be proportional to changes in survivability. For this reason, LVESVI was set as a cardiac function index. Regarding the degree of improvement in LVESVI, considering that a reduction of 10 or more is reportedly used as a criterion for cardiac resynchronization therapy responder, and in consideration of measurement errors, fluctuations within 10% were defined as “constant.”
[2] LVEDVI
[3] LVDs
[4] LVDd
[5] Changes in CTR

### (4) Changes in Heart Failure Symptoms Before and After Attachment

The following items were set to evaluate the severity of heart failure and the improvement of symptoms.

### [1] NYHA Classification

The NYHA classification was set as an endpoint to examine the improvement of the severity of heart failure.

### [2] 6-Minute Walking Distance

The 6-minute walking distance was set as an index of QOL because it is often used as a simple method to measure exercise tolerance.

### (5) Changes in Myocardial Blood Flow; Ammonia PET Study

This is a useful evaluation method for confirming the recovery of blood flow in the ischemic local area (where the test drug is attached).

13NH3: By intravenously administering ammonia, the ammonia gathers in the heart in response to blood flow, and blood flow and movement in the local area of the heart can be known by examining the degree of ammonia gathering by PET/CT.

### (6) Changes in Brain Natriuretic Peptide (BNP)

BNP was set because it is very useful and widely used as a clinical index of heart failure. Changes in blood BNP from before attachment to 26 weeks after attachment were evaluated.

### (7) QOL Assessment

This was set to assess the QOL of subjects after heart attachment. The patient's QOL status was assessed using the Japanese version of the SF-36 (Version 2) questionnaire.

Table 4 outlines the secondary and exploratory endpoints.

TABLE 4

| | Secondary endpoint | Summary of endpoint | Measurement method |
|---|---|---|---|
| (1) | Plasma drug concentration | Pharmacokinetic parameter (Cₘₐₓ) after | |
| | | attachment | |
| | Exploratory endpoints | Summary of endpoints | Measurement method |
| (1) | Amount of change in left | Changes in left ventricular ejection fraction | Echocardiography |
| | ventricular ejection fraction | (LVEF %) before and after heart attachment | |
| | (LVEF) | | |
| (2) | Changes in left ventricular | Changes in cardiac index (CI) before and after | CT |
| | pump function | heart attachment | |
| (3) | Changes in left ventricular | (i) Changes in left ventricular end-systolic | CT |
| | remodeling | volume index (LVESVI) before and after heart | |
| | | attachment and degree of improvement | |
| | | [increase/constant/decrease] | |
| | | (ii) Changes in left ventricular end-diastolic | CT |
| | | volume index (LVEDVI) before and after heart | |
| | | attachment | |
| | | (iii) Changes in left ventricular end-systolic | Echocardiography |
| | | internal diameter (LVDs) before and after heart | |
| | | attachment | |
| | | (iv) Changes in left ventricular end-diastolic | Echocardiography |
| | | internal diameter (LVDd) before and after heart | |
| | | attachment | |
| | | (v) Changes in cardiothoracic ratio (CTR) | Chest X-ray |
| | | before and after heart attachment | |
| (4) | Changes in heart failure | (i) Changes in NYHA classification before and | |
| | symptoms | after heart attachment | |
| | | (ii) Changes in 6-minute walking distance | |
| | | before and after heart attachment | |
| (5) | Changes in myocardial blood | Changes in myocardial blood flow before and | Ammonia PET |
| | flow | after heart attachment | |
| (6) | Changes in BNP | Changes in blood BNP concentration | |
| (7) | QOL assessment | Change in SF-36 | |

Table 5 shows the schedule of observations and examinations of this trial.

TABLE 5

| | | Consent | | | | | | | | | |
|---|---|---|---|---|---|---|---|---|---|---|---|
| | | acquisition/ | | | | | | | | | |
| | | screening | | | | | | | | | |
| | | Within 4 weeks | | | | | | | | | |
| | | before adminis- | | | | | | | | | |
| | | tration of | Adminis- | After administration | | | | | | | At trial |
| Date of observation/ | | investigational | tration | 1 | 1 | 2 | 4 | 6 | 8 | 26 | discon- |
| examination/evaluation | | drug | 0 days | day | week | weeks | weeks | weeks | weeks | weeks | tinuation |
| Allowable period | | −32 to −1 | | — | ±2 | ±6 | ±1 | ±2 | ±2 | ±4 | |
| | | days | | | days | days | week | weeks | weeks | weeks | |
| Consent acquisition | | ◯ | | | | | | | | | |
| Registration | | ◯ | | | | | | | | | |
| Subject background | | ◯ | | | | | | | | | |
| Fundus examination | | ◯*³ | | | | | | | | | |
| Observation of | Vital signs | ◯ | | ◯ | ◯ | ◯ | ◯ | ◯ | ◯ | ◯ | ◯ |
| clinical | Subjective | ◯ | | | ◯ | ◯ | ◯ | ◯ | ◯ | ◯ | ◯ |
| symptoms | symptoms | | | | | | | | | | |
| | Physical | ◯ | | ◯ | ◯ | ◯ | ◯ | ◯ | ◯ | ◯ | ◯ |
| | findings | | | | | | | | | | |
| Blood test | Hematology test | ◯ | | ◯ | ◯ | ◯ | ◯ | ◯ | ◯ | ◯ | ◯ |
| | Blood | ◯ | | ◯ | ◯ | ◯ | ◯ | ◯ | ◯ | ◯ | ◯ |
| | biochemistry test | | | | | | | | | | |
| | Blood | ◯ | | ◯ | ◯ | ◯ | ◯ | ◯ | ◯ | ◯ | ◯ |
| | coagulation | | | | | | | | | | |
| | system test | | | | | | | | | | |
| 12-Lead electrocardiography | | ◯ | | ◯ | ◯ | ◯ | ◯ | ◯ | | ◯ | ◯ |
| Holter electrocardiography | | ◯*³ | | | | | ◯ | ◯ | ◯ | ◯ | ◯ |
| Echocardiography*¹ | | ◯*³ | | | | ◯ | | ◯ | | ◯ | ◯ |
| Chest | Cardiothoracic | ◯*³ | | ◯ | ◯ | ◯ | ◯ | ◯ | | ◯ | ◯ |
| X-ray | ratio (CTR) | | | | | | | | | | |
| Cardiac-gated CT*² | | ◯*³ | | | | ◯ | | | | ◯ | ◯ |
| Ammonia PET | Evaluation | ◯*³ | | | | | | | | | |
| | of myocardial | | | | | | | | | | |
| | blood flow | | | | | | | ◯ | | ◯ | ◯ |
| BNP | | ◯ | | ◯ | ◯ | ◯ | ◯ | ◯ | ◯ | ◯ | ◯ |
| Blood drug concentration | | | | 1, 3, 6, and 24 hours, 7 days, 10 days, 14 days, 28 days | | | | | | | ◯*⁴ |
| | | | | (4 weeks), 6 weeks, and 8 weeks after administration | | | | | | | |
| Changes | Changes in NYHA | ◯ | | | | | | ◯ | | ◯ | ◯ |
| in heart | classification | | | | | | | | | | |
| failure | 6-Minute | ◯ | | | | | | ◯ | | ◯ | ◯ |
| symptoms | walking distance | | | | | | | | | | |
| QOL assessment | SF-36 | ◯ | | | | | | ◯ | | ◯ | ◯ |

| | | | | | |
|---|---|---|---|---|---|
| Presence or absence and degree of | ◯ | ◯ | ◯ | ◯ | ◯ |
| bleeding after attachment (post-operation) | | | | | |

| Combination therapy | → | |
|---|---|---|
| Adverse events | | → |

*¹Left ventricular ejection fraction (LVEF), left ventricular end-diastolic internal diameter (LVDd), left ventricular end-systolic internal diameter (LVDs)
*²Left ventricular end-systolic volume index (LVESVD), left ventricular end-diastolic volume index (LVEDVI), cardiac index (CI)
*³If there is test data within 4 weeks before consent acquisition, it is possible to use that data.
*⁴Conducted only if the trial is discontinued within 8 weeks after administration of the investigational drug.

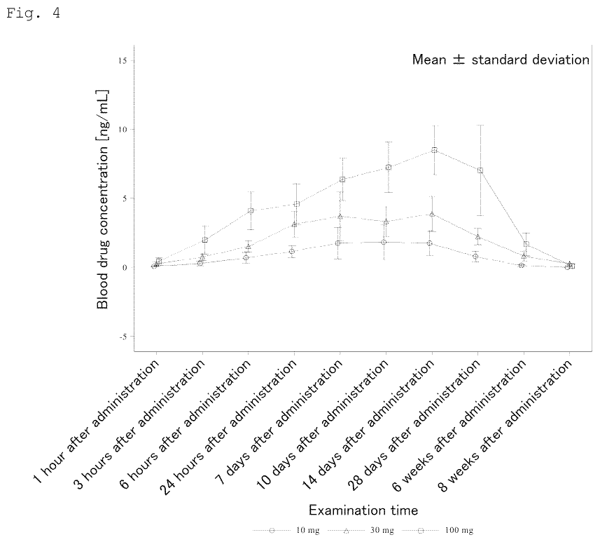

FIG. 3 shows the breakdown of the subjects of this trial. A total of 24 cases were assigned to this trial, with 8 cases each in Group 1, Group 2, and Group 3 (6 cases in the YS-1402 groups and 2 cases in the placebo group). All subjects in Group 1 and Group 2 completed the trial; however, in Group 3, one subject in the YS-1402−100 mg group and one subject in the placebo group were discontinued. The reasons for discontinuation were that the one subject in the YS-1402−100 mg group had “undergone treatment, which was determined to have a significant impact on the results of this trial, by surgical treatment etc. during the trial period,” and that the one subject in the placebo group “stopped coming to the hospital, and examinations and observations could not be performed.”

Table 6 shows the breakdown of the population to be analyzed. Among the enrolled cases, a population excluding unattached cases and subjects with no observed safety data after the investigational drug was attached was defined FAS. Analysis was performed on the FAS.

TABLE 6

| | 10 mg | 30 mg | 100 mg | Placebo |
|---|---|---|---|---|
| Number of cases | 6 | 6 | 6 | 6 |
| assigned | | | | |
| FAS* | 6 (100.0) | 6 (100.0) | 6 (100.0) | 6 (100.0) |

( ): %
*Largest population to be analyzed

YS-1402 dosing sheets were attached to the left ventricle at the end of coronary artery bypass surgery through thoracotomy. Because of the administration at the time of thoracotomy, the attachment frequency was once. At the end of coronary artery bypass surgery, the investigator or sub-investigator confirmed that the investigational drug was properly attached.

### Pharmacokinetic Analysis

Of the FAS, subjects whose blood drug concentration was measured were evaluated by dose group except for the placebo group. ONO-1301, the active ingredient of the investigational drug YS-1402, was the subject of blood concentration measurement.

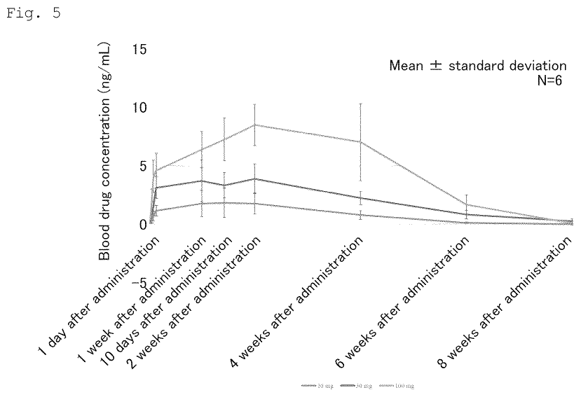

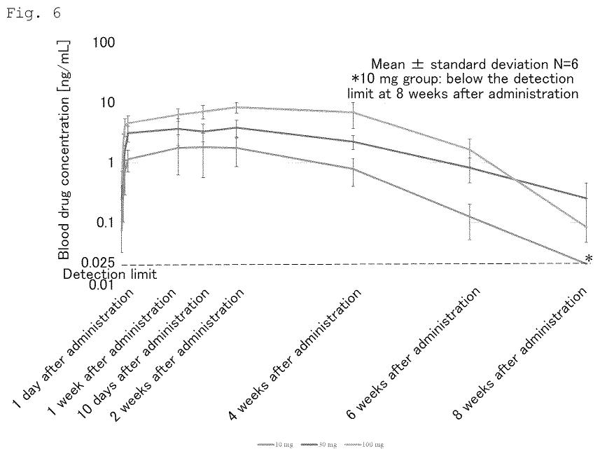

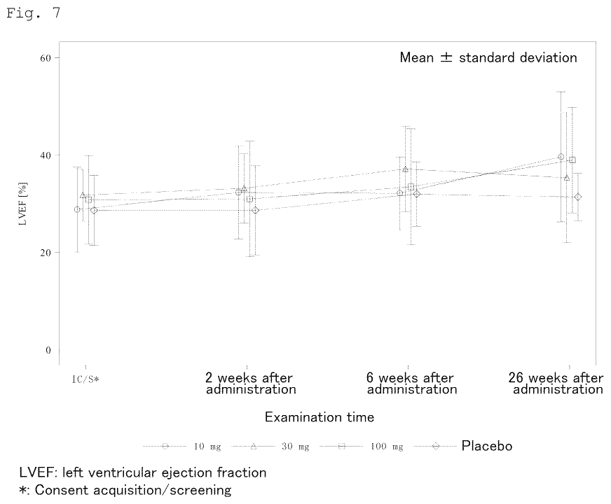

FIG. 4 shows a chart of time-dependent transition of blood ONO-1301 concentration when YS-1402−10 mg, 30 mg, or 100 mg was attached once at the time of thoracotomy for coronary artery bypass surgery [horizontal axis (time: equally spaced at each blood collection point), vertical axis (blood drug concentration: real number, logarithm)]. For reference, FIGS. 5 and 6 show charts of time-dependent transition on the horizontal axis (time: proportional to the actual time) and the vertical axis (blood drug concentration: real number, logarithm). Table 7 shows the summary statistics of the blood ONO-1301 concentration, and Table 8 summarizes the pharmacokinetic parameters of ONO-1301 in blood. In addition, a chart of time-dependent transition of blood ONO-1301 concentration for each subject (vertical axis: real number, logarithm), a list of changes in blood ONO-1301 concentration, and a list of pharmacokinetic parameters were attached in [Appendix 16.2.5].

Regarding the summary statistics of the pharmacokinetic parameters of ONO-1301 in blood, in the order of YS-1402−10 mg group, 30 mg group, and 100 mg group, Cmax (mean±standard deviation, hereinafter the same) was 2.0788±1.1579, 4.2967±1.5310, and 8.8383±2.1971 ng/ml, Tmax was 230.486±87.933, 184.097±143.597, and 419.250±121.598 hours, MRT0-t was 341.856±30.693, 400.176±35.353, and 397.548±34.640 hours, and AUC0-t was 1059.9076±522.3988, 2640.5036±730.4192, and 5572.9516±1190.7685 ng·h/ml. t1/2 (0−4w) of the YS-1402−100 mg group and t1/2 (4w-8w) of all YS-1402 dosing groups could not be calculated.

The blood ONO-1301 concentration increased over time in all of the YS-1402 groups, reached a plateau 7 days after administration in the YS-1402−10 mg group and 30 mg group, and remained high until 28 days after administration. In the 100 mg group, the concentration peaked at 14 days after administration and remained high until 28 days after administration (continually changed from about 4 ng/ml to 9 ng/ml from 24 hours after administration to 28 days after administration).

Cmax and AUC0-t of blood ONO-1301 increased depending on the YS-1402 dose. When Cmax and AUC0-t in the YS-1402−10 mg group were each set to 1, Cmax and AUC0-t in the 30 mg group were 2.07 and 2.49 times, respectively, and similarly 4.25 and 5.26 times in the 100 mg group, which were less than the common ratio. On the other hand, MRT0-t was almost constant. The blood ONO-1301 concentration decreased gradually from day 14 after administration, further decreased sharply from day 28 after administration, and almost disappeared 8 weeks after administration in all dosing groups.

The maximum Cmax value of ONO-1301 in the YS-1402−100 mg group, which was the maximum dose group, was 11.900 ng/ml. At any dose, none exceeded the no-observed-effect level 15.61 ng/ml and the no-observed-adverse-effect level 23.69 ng/ml obtained in oral dosing phase I study.

TABLE 7
Unit: [ng/ml]

| | | | | | | | | | 28 days | | |
|---|---|---|---|---|---|---|---|---|---|---|---|
| | | | | | | | | | after | | |
| | | | | | | | | | adminis- | | |
| | | | | | | | | | tration | | |
| | | | | | | 7 days | 10 days | 14 days | (4 weeks | 6 weeks | 8 weeks |
| | | | | | | after | after | after | after | after | after |
| Dosing | Summary | After administration [time] | | | | adminis- | adminis- | adminis- | adminis- | adminis- | adminis- |
| group | statistics | 1 | 3 | 6 | 24 | tration | tration | tration | tration | tration | tration |
| 10 mg | Number of | 6 | 6 | 6 | 6 | 6 | 6 | 6 | 6 | 6 | 6 |
| | subjects | | | | | | | | | | |
| | Average | 0.07298 | 0.26867 | 0.68350 | 1.14600 | 1.76333 | 1.83200 | 1.75483 | 0.78167 | 0.12522 | 0.00000 |
| | value | | | | | | | | | | |
| | Standard | 0.04176 | 0.16808 | 0.39874 | 0.44653 | 1.13891 | 1.26721 | 0.90063 | 0.38571 | 0.07395 | 0.00000 |
| | deviation | | | | | | | | | | |
| | Minimum | 0.0468 | 0.1290 | 0.2880 | 0.7240 | 0.7900 | 0.7710 | 0.9530 | 0.2220 | 0.0521 | 0.0000 |
| | value | | | | | | | | | | |
| | Median | 0.05185 | 0.21450 | 0.60900 | 1.04500 | 1.43000 | 1.41000 | 1.56500 | 0.86950 | 0.11750 | 0.00000 |
| | value | | | | | | | | | | |
| | Maximum | 0.1540 | 0.5840 | 1.4000 | 2.0000 | 3.9100 | 4.1300 | 3.2600 | 1.2700 | 0.2520 | 0.0000 |
| | value | | | | | | | | | | |
| | Geometric | 0.06583 | 0.23463 | 0.59856 | 1.08674 | 1.52463 | 1.53579 | 1.57940 | 0.67583 | 0.10811 | |
| | mean | | | | | | | | | | |
| | Geometric | 48.9 | 59.0 | 60.8 | 35.3 | 61.8 | 70.3 | 53.1 | 72.7 | 65.9 | |
| | coefficient | | | | | | | | | | |
| | of | | | | | | | | | | |
| | variation | | | | | | | | | | |
| 30 mg | Number of | 6 | 6 | 6 | 6 | 6 | 6 | 6 | 6 | 6 | 6 |
| | subjects | | | | | | | | | | |
| | Average | 0.24023 | 0.74533 | 1.51200 | 3.12833 | 3.70500 | 3.33333 | 3.87500 | 2.23000 | 0.81817 | 0.25078 |
| | value | | | | | | | | | | |
| | Standard | 0.13876 | 0.27000 | 0.40491 | 0.94003 | 1.77246 | 1.09220 | 1.27626 | 0.58378 | 0.36484 | 0.20495 |
| | deviation | | | | | | | | | | |
| | Minimum | 0.0714 | 0.4050 | 0.8820 | 1.5800 | 1.9200 | 2.2200 | 2.2200 | 1.4600 | 0.1910 | 0.0000 |
| | value | | | | | | | | | | |
| | Median | 0.24500 | 0.78400 | 1.54000 | 3.18500 | 3.26500 | 2.98500 | 3.70000 | 2.25000 | 0.90650 | 0.26700 |
| | value | | | | | | | | | | |
| | Maximum | 0.4350 | 1.0200 | 2.0300 | 4.1900 | 6.6700 | 5.2100 | 5.4100 | 2.9800 | 1.1800 | 0.4720 |
| | value | | | | | | | | | | |
| | Geometric | 0.20163 | 0.69999 | 1.46162 | 2.98685 | 3.38275 | 3.19956 | 3.69388 | 2.16368 | 0.70897 | 0.21439 |
| | mean | | | | | | | | | | |
| | Geometric | 78.3 | 41.8 | 30.2 | 36.4 | 48.9 | 31.5 | 35.5 | 27.8 | 76.6 | 168.5 |
| | coefficient | | | | | | | | | | |
| | of | | | | | | | | | | |
| | variation | | | | | | | | | | |
| 100 mg | Number of | 6 | 6 | 6 | 6 | 6 | 6 | 6 | 6 | 6 | 6 |
| | subjects | | | | | | | | | | |
| | Average | 0.43650 | 1.98667 | 4.10000 | 4.57833 | 6.37500 | 7.24167 | 8.48333 | 7.02167 | 1.67333 | 0.08302 |
| | value | | | | | | | | | | |
| | Standard | 0.27822 | 1.03820 | 1.37662 | 1.48535 | 1.53892 | 1.82316 | 1.76189 | 3.28993 | 0.83354 | 0.16680 |
| | deviation | | | | | | | | | | |
| | Minimum | 0.2220 | 1.0700 | 2.3400 | 3.2800 | 4.6600 | 5.4200 | 5.8000 | 2.6500 | 1.1400 | 0.0000 |
| | value | | | | | | | | | | |
| | Median | 0.32200 | 1.68000 | 4.43000 | 4.21000 | 5.84500 | 6.80000 | 8.65500 | 6.80000 | 1.41000 | 0.00000 |
| | value | | | | | | | | | | |
| | Maximum | 0.9320 | 3.9600 | 5.4500 | 7.3600 | 8.6500 | 10.0000 | 10.5000 | 11.9000 | 3.3400 | 0.4170 |
| | value | | | | | | | | | | |
| | Geometric | 0.37729 | 1.80899 | 3.88124 | 4.40854 | 6.22779 | 7.05932 | 8.31932 | 6.31134 | 1.55019 | 0.18390 |
| | mean | | | | | | | | | | |
| | Geometric | 61.9 | 47.8 | 39.0 | 29.7 | 23.8 | 24.9 | 22.4 | 57.1 | 41.2 | 168.0 |
| | coefficient | | | | | | | | | | |
| | of | | | | | | | | | | |
| | variation | | | | | | | | | | |

TABLE 8

| | | | | | t_{1/2} | t_{1/2} | | | AUC₀₋ₜ | AUC_{0-∞} | | |
|---|---|---|---|---|---|---|---|---|---|---|---|---|
| Dosing | Summary | Cmax | Tₘₐₓ | k_{d} | (0-4 W) | (4-8 W) | MRT₀₋ₜ | MRT_{0-∞} | [ng · | [ng · | Cl₀₋ₜ | V_{d} |
| group | statistics | [ng/ml] | [hr] | [1/hr] | [hr] | [hr] | [hr] | [hr] | hr/ml] | hr/ml] | [L/hr] | [L] |
| 10 mg | Number of | 6 | 6 | 4 | 2 | | 6 | 4 | 6 | 4 | 4 | 4 |
| | subjects | | | | | | | | | | | |
| | Average | 2.0788 | 230.486 | 0.00391 | 5006.597 | | 341.856 | 403.229 | 1059.9076 | 1279.1084 | 8.8949 | 2939.1644 |
| | value | | | | | | | | | | | |
| | Standard | 1.1579 | 87.933 | 0.00172 | 6433.561 | | 30.693 | 57.380 | 522.3988 | 545.7913 | 3.5517 | 2186.3127 |
| | deviation | | | | | | | | | | | |
| | Minimum | 0.953 | 112.97 | 0.0024 | 457.38 | | 287.86 | 343.26 | 561.701 | 743.231 | 4.928 | 967.085 |
| | value | | | | | | | | | | | |
| | Median | 1.9850 | 223.158 | 0.00379 | 5006.597 | | 347.461 | 405.922 | 925.7088 | 1172.0908 | 8.5982 | 2569.0512 |
| | value | | | | | | | | | | | |
| | Maximum | 4.130 | 331.00 | 0.0057 | 9555.81 | | 369.61 | 457.81 | 2002.923 | 2029.021 | 13.455 | 5651.470 |
| | value | | | | | | | | | | | |
| 30 mg | Number of | 6 | 6 | 5 | 3 | | 6 | 5 | 6 | 5 | 5 | 5 |
| | subjects | | | | | | | | | | | |
| | Average | 4.2967 | 184.097 | 0.00452 | 1299.195 | | 400.176 | 437.660 | 2640.5036 | 2829.6461 | 11.3668 | 2780.0106 |
| | value | | | | | | | | | | | |
| | Standard | 1.5310 | 143.597 | 0.00139 | 951.379 | | 35.353 | 34.198 | 730.4192 | 752.1549 | 3.7009 | 1558.5734 |
| | deviation | | | | | | | | | | | |
| | Minimum | 2.220 | 23.80 | 0.0032 | 513.39 | | 353.93 | 401.18 | 1626.454 | 1705.351 | 8.050 | 1525.077 |
| | value | | | | | | | | | | | |
| | Median | 4.0200 | 185.067 | 0.00432 | 1027.257 | | 397.455 | 441.823 | 2692.7158 | 2873.2132 | 10.4413 | 2475.1558 |
| | value | | | | | | | | | | | |
| | Maximum | 6.670 | 353.87 | 0.0068 | 2356.93 | | 440.26 | 487.87 | 3663.908 | 3726.649 | 17.592 | 5442.842 |
| | value | | | | | | | | | | | |
| 100 mg | Number of | 6 | 6 | 1 | | | 6 | 1 | 6 | 1 | 1 | 1 |
| | subjects | | | | | | | | | | | |
| | Average | 8.8383 | 419.250 | 0.00600 | | | 397.548 | 436.323 | 5572.9516 | 6165.1201 | 16.2203 | 2702.6758 |
| | value | | | | | | | | | | | |
| | Standard | 2.1971 | 121.598 | | | | 34.640 | | 1190.7685 | | | |
| | deviation | | | | | | | | | | | |
| | Minimum | 5.800 | 312.00 | 0.0060 | | | 350.81 | 436.32 | 4267.141 | 6165.120 | 16.220 | 2702.676 |
| | value | | | | | | | | | | | |
| | Median | 8.7500 | 366.683 | 0.00600 | | | 403.067 | 436.323 | 5631.3118 | 6165.1201 | 16.2203 | 2702.6758 |
| | value | | | | | | | | | | | |
| | Maximum | 11.900 | 591.72 | 0.0060 | | | 433.98 | 436.32 | 7435.915 | 6165.120 | 16.220 | 2702.676 |
| | value | | | | | | | | | | | |

### Analysis of Exploratory Endpoints

(1) Amount of change in left ventricular ejection fraction (LVEF) at 26 weeks after attachment

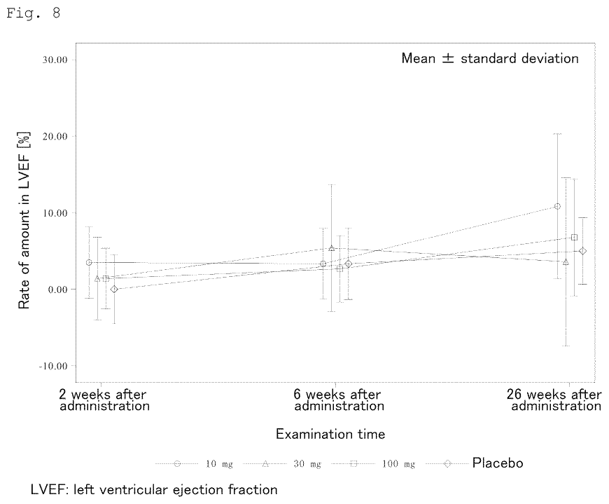

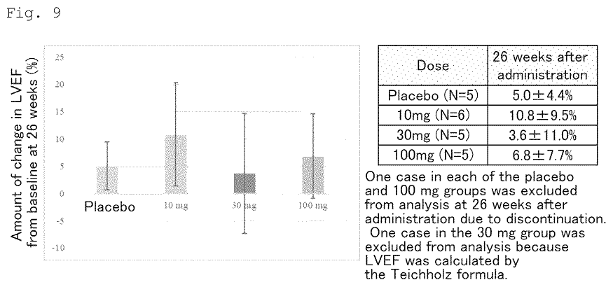

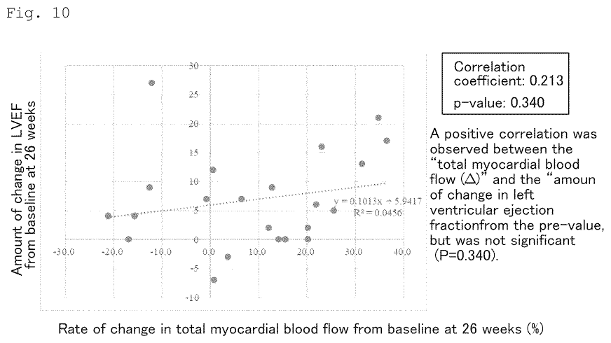

FIG. 7 shows a chart of time-dependent transition of measured LVEF values in echocardiography when YS-1402−10 mg, 30 mg, or 100 mg was attached once at the time of thoracotomy for coronary artery bypass surgery, FIG. 8 shows a chart of time-dependent transition of the amount of change, Table 9 shows the summary statistics of the measured values, Table 10 shows the summary statistics of the amount of change, and Table 11 shows the results of the analysis of variance of repeated measurements. For reference, FIG. 9 shows the amount of change in LVEF from the baseline at 26 weeks after administration, and FIG. 10 shows the correlation between the rate of change in total myocardial blood flow and the amount of change in LVEF from the baseline at 26 weeks after administration of the investigational drug. In addition, a chart of time-dependent transition of the measured LVEF values and the amount of change in LVEF for each subject, and a list of the measured values and the amount of change were attached in [Appendix 16.2.6].

The amount of change in LVEF (mean±standard deviation) was as follows, in the order of the YS-1402−10 mg group, 30 mg group, 100 mg group, and placebo group: 2 weeks after administration of the investigational drug: 3.5±4.7, 1.4±5.4, 1.4±4.0, 0.0±4.5%; 6 weeks: 3.3±4.6, 5.4±8.3, 2.7±4.4, 3.3±4.7%; 26 weeks: 10.8±9.5, 3.6±11.0, 6.8±7.7, 5.0±4.4%. As a result of analysis of variance of repeated measurements with factors of dose group, time of measurement, and dose group×time of measurement, variations in dose group and dose group×time of measurement were not significant; however, only variation in time of measurement was significant. For the two groups, i.e., the active groups combined with three doses of YS-1402 and the placebo group, variations in dose group and dose group×time of measurement were not significant; however, only variation in time of measurement was significant.

The placebo group showed a slight increase over time. The YS-1402 dosing groups showed improvement at 26 weeks after administration; however, there was no dose-related improvement. At 26 weeks, the 10 mg dosing group showed improvement with an amount of change of 5.8% compared to the placebo group. In addition, cardiac function is expected to improve with increased myocardial blood flow. Therefore, the rate of change in total myocardial blood flow and the amount of change in LVEF from the baseline at 26 weeks after administration of the investigational drug were examined. As a result, a positive correlation was observed, but was not significant (p-value: 0.340).

One case (CV-B003) in the YS-1402 30 mg group developed a serious adverse event (congestive heart failure) due to poor medication compliance 26 weeks after administration and 1 week before the test, and LVEF decreased significantly. Therefore, this case was excluded. As a result, the amount of change in LVEF at 26 weeks after administration was from 3.6±110% to 6.3±10.7%.

TABLE 9

| Test | Dosing | Summary | | 2 weeks after | 6 weeks after | 26 weeks after |
|---|---|---|---|---|---|---|
| item | group | statistics | IC/S* | administration | administration | administration |
| LVEF | 10 mg | Number of | 6 | 6 | 6 | 6 |
| (left | | subjects | | | | |
| ventricular | | Average | 28.8 | 32.3 | 32.2 | 39.7 |
| ejection | | value | | | | |
| fraction) | | Standard | 8.7 | 9.5 | 7.5 | 13.4 |
| [%] | | deviation | | | | |
| | | Minimum | 16 | 14 | 19 | 22 |
| | | value | | | | |
| | | Median | 32.5 | 34.5 | 33.5 | 38.0 |
| | | value | | | | |
| | | Maximum | 38 | 41 | 41 | 61 |
| | | value | | | | |
| | 30 mg | Number of | 5 | 5 | 5 | 5 |
| | | subjects | | | | |
| | | Average | 31.8 | 33.2 | 37.2 | 35.4 |
| | | value | | | | |
| | | Standard | 5.3 | 7.2 | 8.8 | 13.4 |
| | | deviation | | | | |
| | | Minimum | 27 | 23 | 23 | 20 |
| | | value | | | | |
| | | Median | 30.0 | 35.0 | 40.0 | 35.0 |
| | | value | | | | |
| | | Maximum | 40 | 40 | 44 | 55 |
| | | value | | | | |
| | 100 mg | Number of | 6 | 5 | 6 | 5 |
| | | subjects | | | | |
| | | Average | 30.8 | 31.0 | 33.5 | 39.0 |
| | | value | | | | |
| | | Standard | 9.1 | 11.9 | 11.9 | 10.8 |
| | | deviation | | | | |
| | | Minimum | 17 | 17 | 20 | 30 |
| | | value | | | | |
| | | Median | 33.0 | 27.0 | 33.0 | 38.0 |
| | | value | | | | |
| | | Maximum | 40 | 48 | 50 | 57 |
| | | value | | | | |
| | Placebo | Number of | 6 | 6 | 6 | 5 |
| | | subjects | | | | |
| | | Average | 28.7 | 28.7 | 32.0 | 31.4 |
| | | value | | | | |
| | | Standard | 7.2 | 9.2 | 6.6 | 4.9 |
| | | deviation | | | | |
| | | Minimum | 20 | 18 | 23 | 24 |
| | | value | | | | |
| | | Median | 27.5 | 26.5 | 33.0 | 32.0 |
| | | value | | | | |
| | | Maximum | 40 | 44 | 40 | 37 |
| | | value | | | | |

*Consent acquisition/screening

TABLE 10

| | Dosing | | 2 weeks after | 6 weeks after | 26 weeks after |
|---|---|---|---|---|---|
| Test item | group | Summary statistics | administration | administration | administration |
| Amount of | 10 mg | Number of subjects | 6 | 6 | 6 |
| change in LVEF | | Average value | 3.5 | 3.3 | 10.8 |
| (left ventricular | | Standard deviation | 4.7 | 4.6 | 9.5 |
| ejection fraction) | | Minimum value | −2 | −2 | 0 |
| [%] | | Median value | 3.0 | 2.5 | 8.0 |
| | | Maximum value | 12 | 12 | 27 |
| | 30 mg | Number of subjects | 5 | 5 | 5 |
| | | Average value | 1.4 | 5.4 | 3.6 |
| | | Standard deviation | 5.4 | 8.3 | 11.0 |
| | | Minimum value | −7 | −7 | −7 |
| | | Median value | 2.0 | 6.0 | 0.0 |
| | | Maximum value | 7 | 16 | 21 |
| | 100 mg | Number of subjects | 5 | 6 | 5 |
| | | Average value | 1.4 | 2.7 | 6.8 |
| | | Standard deviation | 4.0 | 4.4 | 7.7 |
| | | Minimum value | −2 | −2 | 0 |
| | | Median value | 0.0 | 1.5 | 2.0 |
| | | Maximum value | 8 | 10 | 17 |
| | Placebo | Number of subjects | 6 | 6 | 5 |
| | | Average value | 0.0 | 3.3 | 5.0 |
| | | Standard deviation | 4.5 | 4.7 | 4.4 |
| | | Minimum value | −6 | −1 | 0 |
| | | Median value | −0.5 | 1.5 | 4.0 |
| | | Maximum value | 6 | 11 | 12 |

TABLE 11

| | | | Analysis of variance table | | | |
|---|---|---|---|---|---|---|
| | | | | Degrees of | | |
| Test | Dosing | Number of | | freedom | F- | p- |
| item | group | subjects | Factor | (numerator) | value | value |
| Amount of | 10 mg | 6 | Dosing group | 3 | 0.33 | 0.8012 |
| change in | 30 mg | 5 | Time of | 2 | 11.01 | 0.0011 |
| LVEF (left | | | measurement | | | |
| ventricular | 100 mg | 6 | Dosing group × | 6 | 0.78 | 0.5955 |
| ejection | | | time of | | | |
| fraction) [%] | | | measurement | | | |
| | Placebo | 6 | | | | |
| | Active group* | 17 | Dosing group | 1 | 0.21 | 0.6541 |
| | Placebo | 6 | Time of | 2 | 11.36 | 0.0007 |
| | | | measurement | | | |
| | | | Dosing group × | 2 | 0.48 | 0.6292 |
| | | | time of | | | |
| | | | measurement | | | |

*Combined with 10 mg, 30 mg, and 100 mg dosing groups

### (2) Changes in Left Ventricular Pump Function (Cardiac Index [CI]) Before and After Attachment

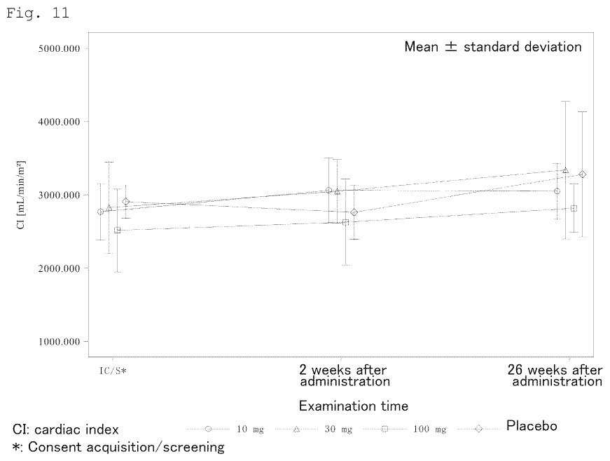

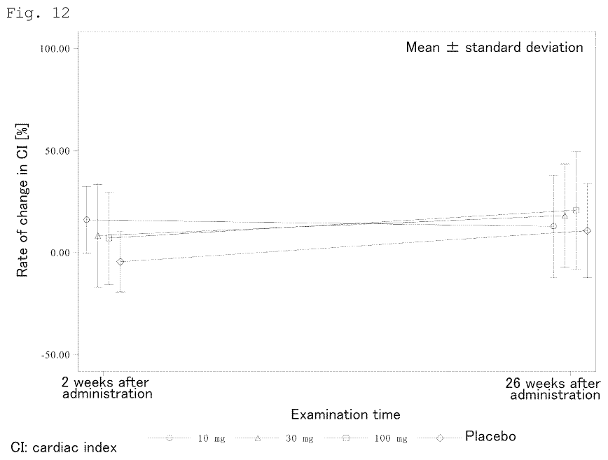

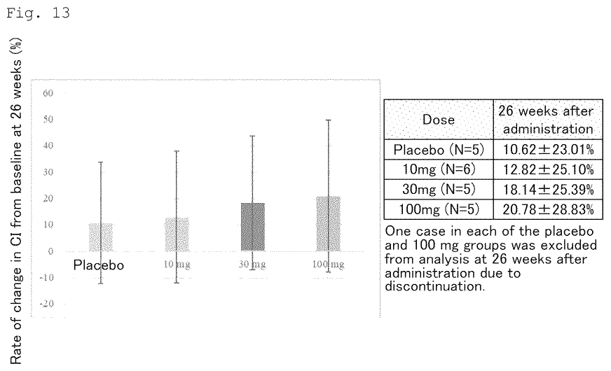

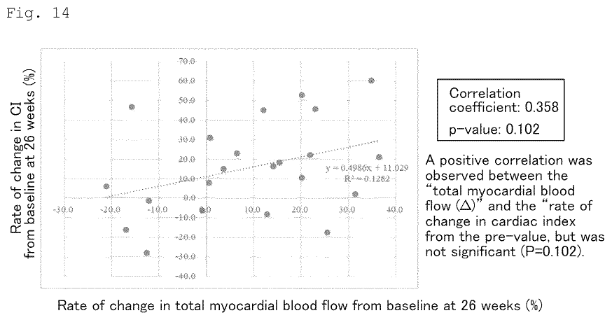

FIG. 11 shows a chart of time-dependent transition of measured CI values in cardiac-gated CT, FIG. 12 shows a chart of time-dependent transition of the rate of change, Table 12 shows the summary statistics of the measured values, Table 13 shows the summary statistics of the rate of change, and Table 14 shows the results of the analysis of variance of repeated measurements. For reference, FIG. 13 shows the rate of change in CI from the baseline at 26 weeks after administration, and FIG. 14 shows the correlation between the rate of change in total myocardial blood flow and the rate of change in CI from the baseline at 26 weeks after administration of the investigational drug. In addition, a chart of time-dependent transition of the measured CI values and the rate of change for each subject, and a list of the measured values and the amount of change were attached in [Appendix 16.2.6].

The rate of change in CI (mean±standard deviation) was as follows, in the order of the YS-1402−10 mg group, 30 mg group, 100 mg group, and placebo group:

2 weeks after administration of the investigational drug: 16.03±16.34, 8.22±25.27, 6.99±22.64, −4.56±14.84%; 26 weeks: 12.82±25.10, 18.14±25.39, 20.78±28.83, 10.62±23.01%.

The analysis of variance of repeated measurements with factors of dose group, time of measurement, and dose group×time of measurement did not show any significance. The results were similar for the two groups: the active groups combined with three doses of YS-1402 and the placebo group.

The placebo group showed a decrease at 2 weeks after administration, but showed an increase at 26 weeks. The YS-1402 dosing groups generally showed an increase over time, and showed improvement at 26 weeks compared to the placebo group, confirming a dose-related increase. At 26 weeks after administration, the 100 mg group showed improvement with a rate of change of 10.16% s compared to the placebo group. From the above, CI increased in a dose-related manner in the order of the placebo group, YS-1402−10 mg group, 30 mg group, and 100 mg group at 26 weeks after administration. In addition, a positive correlation was observed between the rate of change in total myocardial blood flow and the rate of change in CI from the baseline at 26 weeks after administration of the investigational drug; however, the correlation was not significant (p-value: 0.102).

TABLE 12

| | Dosing | | | 2 weeks after | 26 weeks after |
|---|---|---|---|---|---|
| Test item | group | Summary statistics | IC/S* | administration | administration |
| CI | 10 mg | Number of subjects | 6 | 5 | 6 |
| (cardiac index) | | Average value | 2769.100 | 3060.968 | 3049.405 |
| [mL/min/m²] | | Standard deviation | 383.107 | 443.657 | 380.462 |
| | | Minimum value | 2338.27 | 2522.89 | 2412.16 |
| | | Median value | 2752.330 | 3003.220 | 3031.520 |
| | | Maximum value | 3353.74 | 3760.26 | 3464.11 |
| | 30 mg | Number of subjects | 6 | 5 | 6 |
| | | Average value | 2826.928 | 3047.230 | 3338.473 |
| | | Standard deviation | 623.652 | 435.135 | 941.260 |
| | | Minimum value | 2320.81 | 2320.81 | 2169.87 |
| | | Median value | 2496.425 | 3142.460 | 3594.830 |
| | | Maximum value | 3637.25 | 3477.96 | 4264.81 |
| | 100 mg | Number of subjects | 6 | 6 | 5 |
| | | Average value | 2514.588 | 2629.082 | 2818.050 |
| | | Standard deviation | 563.446 | 584.668 | 327.177 |
| | | Minimum value | 1824.04 | 1860.60 | 2397.65 |
| | | Median value | 2595.050 | 2687.455 | 2812.150 |
| | | Maximum value | 3200.23 | 3259.18 | 3259.64 |
| | Placebo | Number of subjects | 6 | 6 | 5 |
| | | Average value | 2907.997 | 2761.382 | 3278.670 |
| | | Standard deviation | 226.640 | 370.014 | 852.573 |
| | | Minimum value | 2697.24 | 2323.51 | 2313.91 |
| | | Median value | 2822.345 | 2826.155 | 3000.680 |
| | | Maximum value | 3236.49 | 3322.00 | 4607.95 |

*Consent acquisition/screening

TABLE 13

| | Dosing | | 2 weeks after | 26 weeks after |
|---|---|---|---|---|
| Test item | group | Summary statistics | administration | administration |
| Rate of | 10 mg | Number of subjects | 5 | 6 |
| change in CI | | Average value | 16.03 | 12.82 |
| (cardiac index) | | Standard deviation | 16.34 | 25.10 |
| [%] | | Minimum value | −11.2 | −28.1 |
| | | Median value | 24.86 | 19.16 |
| | | Maximum value | 28.4 | 45.6 |
| | 30 mg | Number of subjects | 5 | 6 |
| | | Average value | 8.22 | 18.14 |
| | | Standard deviation | 25.27 | 25.39 |
| | | Minimum value | −15.3 | −8.4 |
| | | Median value | 0.00 | 16.53 |
| | | Maximum value | 45.1 | 59.9 |
| | 100 mg | Number of subjects | 6 | 5 |
| | | Average value | 6.99 | 20.78 |
| | | Standard deviation | 22.64 | 28.83 |
| | | Minimum value | −35.1 | −16.4 |
| | | Median value | 11.99 | 21.05 |
| | | Maximum value | 26.5 | 52.5 |
| | Placebo | Number of subjects | 6 | 5 |
| | | Average value | −4.56 | 10.62 |
| | | Standard deviation | 14.84 | 23.01 |
| | | Minimum value | −24.4 | −17.6 |
| | | Median value | −5.88 | 7.81 |
| | | Maximum value | 17.1 | 46.5 |

TABLE 14

| | | | Analysis of variance table | | | |
|---|---|---|---|---|---|---|
| Test | Dosing | Number of | | Degrees of freedom | F- | p- |
| item | group | subjects | Factor | (numerator) | value | value |
| Rate of | 10 mg | 6 | Dosing group | 3 | 0.33 | 0.8017 |
| change in | 30 mg | 6 | Time of | 1 | 3.89 | 0.0655 |
| CI (cardiac | | | measurement | | | |
| index) [%] | 100 mg | 6 | Dosing group × | 3 | 0.48 | 0.6981 |
| | | | time of | | | |
| | | | measurement | | | |
| | Placebo | 6 | | | | |
| | Active group* | 18 | Dosing group | 1 | 1.01 | 0.3250 |
| | Placebo | 6 | Time of | 1 | 4.30 | 0.0527 |
| | | | measurement | | | |
| | | | Dosing group × | 1 | 0.43 | 0.5205 |
| | | | time of | | | |
| | | | measurement | | | |

*Combined with 10 mg, 30 mg, and 100 mg dosing groups

(3) Changes in left ventricular remodeling before and after attachment
1) Changes in left ventricular end-systolic volume index (LVESVI) before and after heart attachment and degree of improvement [increase/constant/decrease]

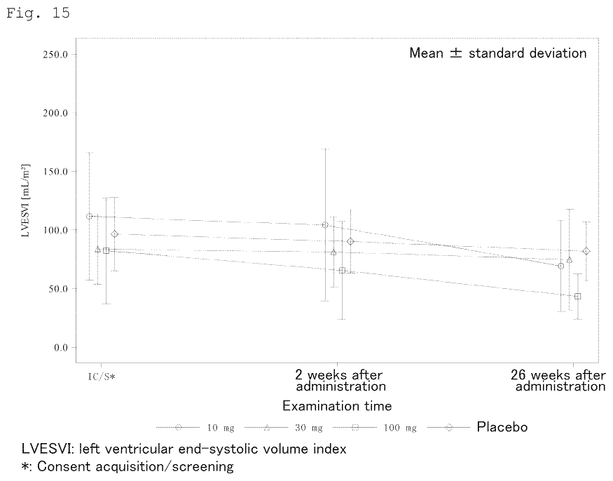

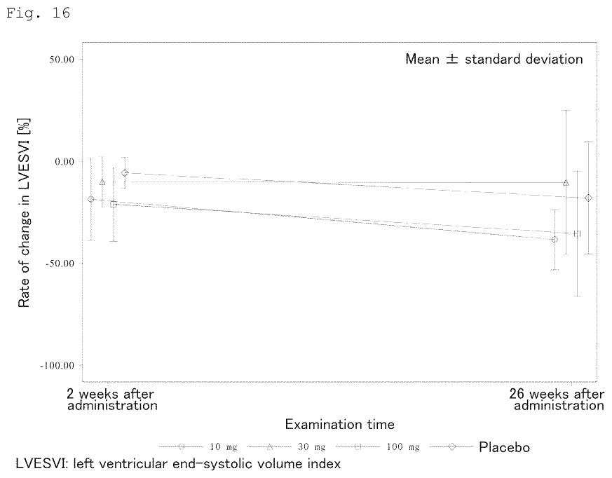

FIG. 15 shows a chart of time-dependent transition of measured LVESVI values in cardiac-gated CT, FIG. 16 shows a chart of time-dependent transition of the rate of change, Table 15 shows the summary statistics of the measured values, Table 16 shows the summary statistics of the rate of change, and Table 17 shows the results of the analysis of variance of repeated measurements. In addition, a chart of time-dependent transition of the measured LVESVI values and the amount of change for each subject, and a list of the measured values and the amount of change were attached in [Appendix 16.2.6].

The rate of change in LVESVI (mean±standard deviation) was as follows, in the order of the YS-1402−10 mg group, 30 mg group, 100 mg group, and placebo group:

2 weeks after administration of the investigational drug: −18.68±20.22, −10.09±12.32, −21.18±18.11, −5.70±7.60%; 26 weeks: −38.49±14.79, −10.48±35.42, −35.51±30.81, −18.03±27.42%.

The analysis of variance of repeated measurements with factors of dose group, time of measurement, and dose group×time of measurement did not show any significance. The results were similar for the two groups: the active groups combined with three doses of YS-1402 and the placebo group.

The placebo group showed a decrease over time. The YS-1402 groups also showed a decrease over time. In particular, the degree of decrease in the YS-1402 groups at 2 weeks was greater than in the placebo group, but was not in response to the dose. The degree of improvement was examined in 11.4.7.2 conclusion of exploratory endpoints.

TABLE 15

| | Dosing | | | 2 weeks after | 26 weeks after |
|---|---|---|---|---|---|
| Test item | group | Summary statistics | IC/S* | administration | administration |
| LVESVI (left | 10 mg | Number of subjects | 6 | 5 | 6 |
| ventricular | | Average value | 111.5 | 104.2 | 69.2 |
| end-systolic | | Standard deviation | 54.5 | 64.8 | 38.8 |
| volume index) | | Minimum value | 56 | 50 | 34 |
| [mL/m²] | | Median value | 96.5 | 100.0 | 56.0 |
| | | Maximum value | 207 | 212 | 127 |
| | 30 mg | Number of subjects | 6 | 5 | 6 |
| | | Average value | 83.5 | 81.2 | 74.7 |
| | | Standard deviation | 29.8 | 29.9 | 43.2 |
| | | Minimum value | 51 | 59 | 35 |
| | | Median value | 73.0 | 69.0 | 66.0 |
| | | Maximum value | 130 | 130 | 158 |
| | 100 mg | Number of subjects | 6 | 6 | 5 |
| | | Average value | 82.2 | 65.5 | 43.2 |
| | | Standard deviation | 45.2 | 41.9 | 19.3 |
| | | Minimum value | 36 | 29 | 14 |
| | | Median value | 66.0 | 45.5 | 52.0 |
| | | Maximum value | 151 | 133 | 62 |
| | Placebo | Number of subjects | 6 | 6 | 5 |
| | | Average value | 96.3 | 90.0 | 81.8 |
| | | Standard deviation | 31.1 | 27.1 | 25.1 |
| | | Minimum value | 65 | 66 | 46 |
| | | Median value | 86.5 | 79.5 | 97.0 |
| | | Maximum value | 140 | 127 | 104 |

*Consent acquisition/screening

TABLE 16

| | Dosing | | 2 weeks after | 26 weeks after |
|---|---|---|---|---|
| Test item | group | Summary statistics | administration | administration |
| Rate of change | 10 mg | Number of subjects | 5 | 6 |
| in LVESVI (left | | Average value | −18.68 | −38.49 |
| ventricular | | Standard deviation | 20.22 | 14.79 |
| end-systolic | | Minimum value | −46.2 | −63.4 |
| volume index) | | Median value | −22.97 | −38.07 |
| [%] | | Maximum value | 2.4 | −23.0 |
| | 30 mg | Number of subjects | 5 | 6 |
| | | Average value | −10.09 | −10.48 |
| | | Standard deviation | 12.32 | 35.42 |
| | | Minimum value | −21.3 | −50.0 |
| | | Median value | −16.90 | −18.50 |
| | | Maximum value | 6.2 | 45.0 |
| | 100 mg | Number of subjects | 6 | 5 |
| | | Average value | −21.18 | −35.51 |
| | | Standard deviation | 18.11 | 30.81 |
| | | Minimum value | −39.7 | −64.2 |
| | | Median value | −25.95 | −46.03 |
| | | Maximum value | 7.3 | 4.0 |
| | Placebo | Number of subjects | 6 | 5 |
| | | Average value | −5.70 | −18.03 |
| | | Standard deviation | 7.60 | 27.42 |
| | | Minimum value | −16.3 | −37.0 |
| | | Median value | −7.01 | −30.71 |
| | | Maximum value | 3.1 | 29.3 |

TABLE 17

| | | | Analysis of variance table | | | |
|---|---|---|---|---|---|---|
| | | | | Degrees of | | |
| Test | Dosing | Number of | | freedom | F- | p- |
| item | group | subjects | Factor | (numerator) | value | value |
| Rate of | 10 mg | 6 | Dosing group | 3 | 1.71 | 0.2001 |
| change in | 30 mg | 6 | Time of | 1 | 2.99 | 0.1009 |
| LVESVI (left | | | measurement | | | |
| ventricular | 100 mg | 6 | Dosing group × | 3 | 0.47 | 0.7093 |
| end-systolic | | | time of | | | |
| volume | | | measurement | | | |
| index) [%] | Placebo | 6 | | | | |
| | Active group* | 18 | Dosing group | 1 | 1.35 | 0.2584 |
| | Placebo | 6 | Time of | 1 | 2.11 | 0.1620 |
| | | | measurement | | | |
| | | | Dosing group × | 1 | 0.01 | 0.9182 |
| | | | time of | | | |
| | | | measurement | | | |

*Combined with 10 mg, 30 mg, and 100 mg dosing groups

2) Changes in left ventricular end-diastolic volume index (LVEDVI) before and after heart attachment

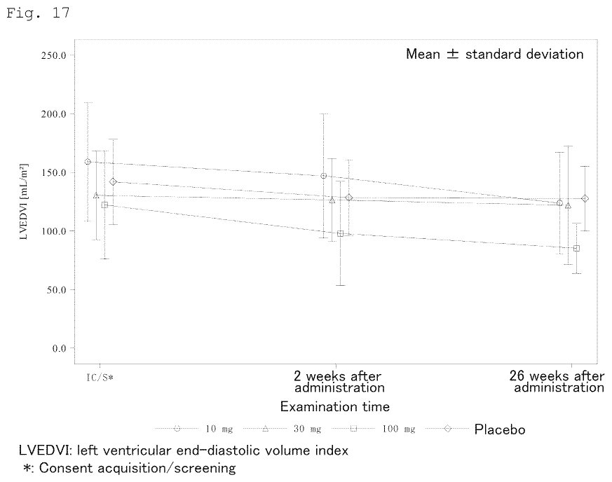

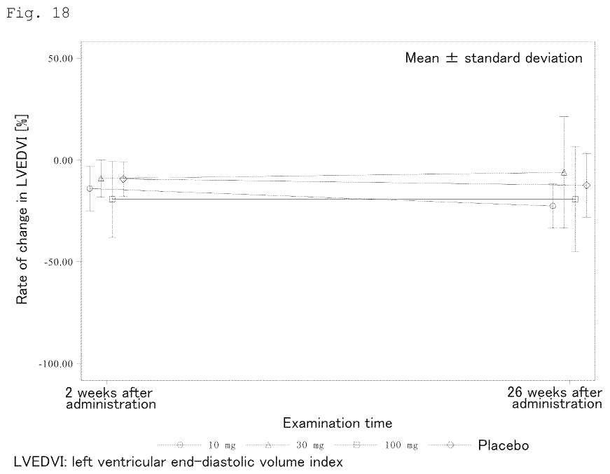

FIG. 17 shows a chart of time-dependent transition of measured LVESVI values in cardiac-gated CT, FIG. 18 shows a chart of time-dependent transition of the rate of change, Table 18 shows the summary statistics of the measured values, Table 19 shows the summary statistics of the rate of change, and Table 20 shows the results of the analysis of variance of repeated measurements. In addition, a chart of time-dependent transition of the measured LVEDVI values and the amount of change for each subject, and a list of the measured values and the amount of change were attached in [Appendix 16.2.6].

The rate of change in LVEDVI (mean±standard deviation) was as follows, in the order of the YS-1402−10 mg group, 30 mg group, 100 mg group, and placebo group:

2 weeks after administration of the investigational drug: −14.00±10.99, −8.98±9.16, −19.30±18.78, −9.30±8.52′; 26 weeks: −22.56±10.73, −5.98±27.50, −19.30±25.82, −12.41±15.73c.
The analysis of variance of repeated measurements with factors of dose group, time of measurement, and dose group×time of measurement did not show any significance. The results were similar for the two groups: the active groups combined with three doses of YS-1402 and the placebo group.

The placebo group showed a decrease over time. The YS-1402 groups generally showed a similar decrease over time; however, the decrease was not in response to the dose.

TABLE 18

| | Dosing | | | 2 weeks after | 26 weeks after |
|---|---|---|---|---|---|
| Test item | group | Summary statistics | IC/S* | administration | administration |
| LVEDVI (left | 10 mg | Number of subjects | 6 | 5 | 6 |
| ventricular | | Average value | 159.0 | 147.0 | 123.7 |
| end-diastolic | | Standard deviation | 50.5 | 53.2 | 43.4 |
| volume index) | | Minimum value | 107 | 103 | 72 |
| [mL/m²] | | Median value | 144.5 | 135.0 | 109.5 |
| | | Maximum value | 246 | 235 | 188 |
| | 30 mg | Number of subjects | 6 | 5 | 6 |
| | | Average value | 130.3 | 126.4 | 121.8 |
| | | Standard deviation | 37.9 | 35.3 | 50.4 |
| | | Minimum value | 87 | 102 | 62 |
| | | Median value | 118.0 | 107.0 | 113.0 |
| | | Maximum value | 184 | 184 | 213 |
| | 100 mg | Number of subjects | 6 | 6 | 5 |
| | | Average value | 122.2 | 97.8 | 85.2 |
| | | Standard deviation | 46.1 | 44.3 | 21.3 |
| | | Minimum value | 68 | 63 | 55 |
| | | Median value | 108.5 | 75.0 | 95.0 |
| | | Maximum value | 190 | 170 | 105 |
| | Placebo | Number of subjects | 6 | 6 | 5 |
| | | Average value | 141.8 | 128.3 | 127.6 |
| | | Standard deviation | 36.3 | 32.4 | 27.5 |
| | | Minimum value | 105 | 85 | 96 |
| | | Median value | 135.5 | 124.5 | 141.0 |
| | | Maximum value | 201 | 175 | 158 |

*Consent acquisition/screening

TABLE 19

| | Dosing | | 2 weeks after | 26 weeks after |
|---|---|---|---|---|
| Test item | group | Summary statistics | administration | administration |
| Rate of change | 10 mg | Number of subjects | 5 | 6 |
| in LVEDVI (left | | Average value | −14.00 | −22.56 |
| ventricular | | Standard deviation | 10.99 | 10.73 |
| end-diastolic | | Minimum value | −28.3 | −34.2 |
| volume index) | | Median value | −17.60 | −24.20 |
| [%] | | Maximum value | −1.5 | −7.2 |
| | 30 mg | Number of subjects | 5 | 6 |
| | | Average value | −8.98 | −5.98 |
| | | Standard deviation | 9.16 | 27.50 |
| | | Minimum value | −18.9 | −43.5 |
| | | Median value | −10.53 | −5.70 |
| | | Maximum value | 0.9 | 26.0 |
| | 100 mg | Number of subjects | 6 | 5 |
| | | Average value | −19.30 | −19.30 |
| | | Standard deviation | 18.78 | 25.82 |
| | | Minimum value | −46.6 | −50.0 |
| | | Median value | −22.20 | −19.12 |
| | | Maximum value | 3.7 | 6.4 |
| | Placebo | Number of subjects | 6 | 5 |
| | | Average value | −9.30 | −12.41 |
| | | Standard deviation | 8.52 | 15.73 |
| | | Minimum value | −19.0 | −29.9 |
| | | Median value | −9.52 | −14.02 |
| | | Maximum value | 2.4 | 11.8 |

TABLE 20

| | | | | Analysis of variance table | | | |
|---|---|---|---|---|---|---|---|
| | | | | | Degrees of | | |
| | Dosing | | Number of | | freedom | F- | p- |
| Test item | group | | subjects | Factor | (numerator) | value | value |
| Rate of change in | 10 | mg | 6 | Dosing group | 3 | 0.94 | 0.4420 |
| LVEDVI (left | 30 | mg | 6 | Time of | 1 | 0.10 | 0.7606 |
| ventricular end- | | | | measurement | | | |
| diastolic volume | 100 | mg | 6 | Dosing group × | 3 | 0.30 | 0.8269 |
| index) [%] | | | | time of | | | |
| | | | | measurement | | | |

| | | | | | |
|---|---|---|---|---|---|
| Placebo | 6 | | | | |
| Active | 18 | Dosing group | 1 | 0.43 | 0.5209 |
| group* | | | | | |
| Placebo | 6 | Time of | 1 | 0.08 | 0.7820 |

| | | | |
|---|---|---|---|
| measurement | | | |
| Dosing group × | 1 | 0.03 | 0.8601 |
| time of | | | |
| measurement | | | |

*Combined with 10 mg, 30 mg, and 100 mg dosing groups

3) Changes in left ventricular end-systolic internal diameter (LVDs) before and after heart attachment

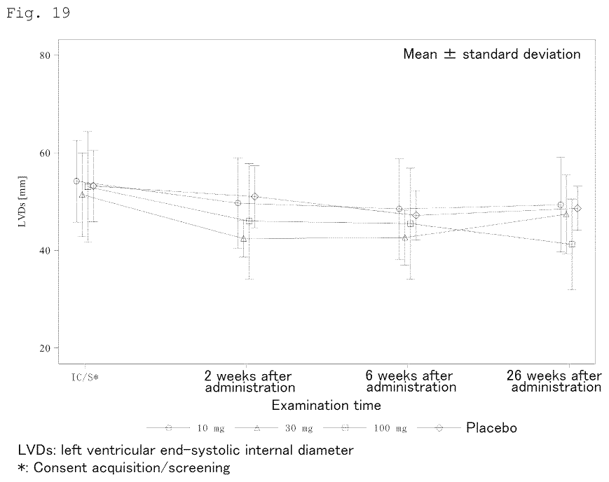

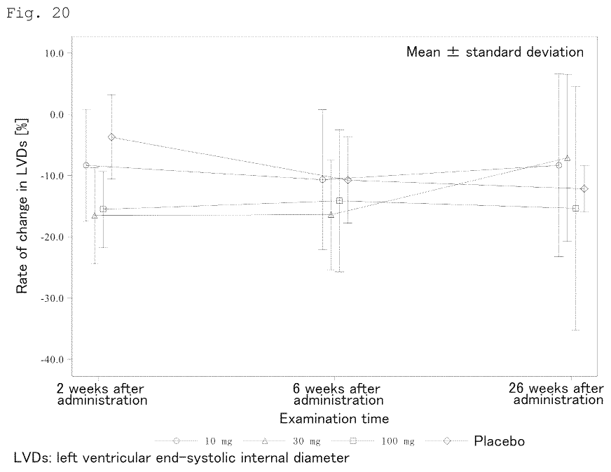

FIG. 19 shows a chart of time-dependent transition of measured LVDs values in echocardiography, FIG. 20 shows a chart of time-dependent transition of the rate of change, Table 21 shows the summary statistics of the measured values, Table 22 shows the summary statistics of the rate of change, and Table 23 shows the results of the analysis of variance of repeated measurements. In addition, a chart of time-dependent transition of the measured LVDs values and the rate of change for each subject, and a list of the measured values and the rate of change were attached in [Appendix 16.2.6].

The rate of change in LVDs (mean±standard deviation) was as follows, in the order of the YS-1402−10 mg group, 30 mg group, 100 mg group, and placebo group:

2 weeks after administration of the investigational drug: −8.33±9.16, −16.56±7.86, −15.51±6.29, −3.70±6.89%; 6 weeks: −10.67±11.43, −16.41±8.95, −14.13±11.60, −10.74±7.06%;
26 weeks: −8.33±14.92, −7.12±13.65, −15.34±19.89, −12.15±3.76%.
The analysis of variance of repeated measurements with factors of dose group, time of measurement, and dose group×time of measurement did not show any significance. Regarding the two groups, i.e., the active groups combined with three doses of YS-1402 and the placebo group, variations in dose group and dose group×time of measurement were not significant; however, only variation in time of measurement was significant.

The placebo group showed a decrease over time. The YS-1402 groups showed a greater decrease than the placebo group at 2 weeks after administration; however, the decrease was not in response to the dose. The decrease was not temporal or dose-dependent at 6 weeks and 26 weeks after administration.

TABLE 21

| | Dosing | | | 2 weeks after | 6 weeks after | 26 weeks after |
|---|---|---|---|---|---|---|
| Test item | group | Summary statistics | IC/S* | administration | administration | administration |
| LVDs (left | 10 mg | Number of subjects | 6 | 6 | 6 | 6 |
| ventricular | | Average value | 54.2 | 49.7 | 48.5 | 49.3 |
| end-systolic | | Standard deviation | 8.4 | 9.2 | 10.3 | 9.7 |
| internal | | Minimum value | 40 | 36 | 34 | 40 |
| diameter) | | Median value | 55.0 | 49.0 | 47.0 | 47.5 |
| [mm] | | Maximum value | 64 | 61 | 62 | 65 |
| | 30 mg | Number of subjects | 5 | 5 | 5 | 5 |
| | | Average value | 51.4 | 42.4 | 42.6 | 47.4 |
| | | Standard deviation | 8.6 | 3.8 | 5.6 | 8.1 |
| | | Minimum value | 41 | 38 | 36 | 35 |
| | | Median value | 51.0 | 44.0 | 40.0 | 48.0 |
| | | Maximum value | 63 | 47 | 49 | 57 |
| | 100 mg | Number of subjects | 6 | 5 | 6 | 5 |
| | | Average value | 53.0 | 46.0 | 45.5 | 41.2 |
| | | Standard deviation | 11.3 | 11.9 | 11.4 | 9.3 |
| | | Minimum value | 40 | 32 | 30 | 29 |
| | | Median value | 49.5 | 48.0 | 44.0 | 44.0 |
| | | Maximum value | 72 | 63 | 65 | 50 |
| | Placebo | Number of subjects | 6 | 6 | 6 | 5 |
| | | Average value | 53.2 | 51.0 | 47.2 | 48.6 |
| | | Standard deviation | 7.3 | 6.4 | 5.0 | 4.5 |
| | | Minimum value | 42 | 42 | 40 | 42 |
| | | Median value | 56.5 | 52.5 | 48.0 | 49.0 |
| | | Maximum value | 59 | 59 | 53 | 54 |

*Consent acquisition/screening

TABLE 22

| | Dosing | | 2 weeks after | 6 weeks after | 26 weeks after |
|---|---|---|---|---|---|
| Test item | group | Summary statistics | administration | administration | administration |
| Rate of change | 10 mg | Number of subjects | 6 | 6 | 6 |
| in LVDs (left | | Average value | −8.33 | −10.67 | −8.33 |
| ventricular | | Standard deviation | 9.16 | 11.43 | 14.92 |
| end-systolic | | Minimum value | −18.8 | −21.9 | −25.0 |
| internal | | Median value | −9.90 | −14.36 | −7.25 |
| diameter) | | Maximum value | 7.0 | 8.8 | 14.0 |
| [%] | 30 mg | Number of subjects | 5 | 5 | 5 |
| | | Average value | −16.56 | −16.41 | −7.12 |
| | | Standard deviation | 7.86 | 8.95 | 13.65 |
| | | Minimum value | −25.4 | −23.8 | −23.8 |
| | | Median value | −17.39 | −21.57 | −9.80 |
| | | Maximum value | −4.9 | −2.4 | 10.9 |
| | 100 mg | Number of subjects | 5 | 6 | 5 |
| | | Average value | −15.51 | −14.13 | −15.34 |
| | | Standard deviation | 6.29 | 11.60 | 19.89 |
| | | Minimum value | −20.8 | −30.0 | −43.3 |
| | | Median value | −18.33 | −11.09 | −8.33 |
| | | Maximum value | −5.9 | 2.1 | 6.4 |
| | Placebo | Number of subjects | 6 | 6 | 5 |
| | | Average value | −3.70 | −10.74 | −12.15 |
| | | Standard deviation | 6.89 | 7.06 | 3.76 |
| | | Minimum value | −11.9 | −17.9 | −17.5 |
| | | Median value | −4.40 | −11.78 | −12.50 |
| | | Maximum value | 7.1 | 2.4 | −8.5 |

TABLE 23

| | | | | Analysis of variance table | | | |
|---|---|---|---|---|---|---|---|
| | | | | | Degrees of | | |
| | Dosing | | Number of | | freedom | F- | p- |
| Test item | group | | subjects | Factor | (numerator) | value | value |
| Rate of change | 10 | mg | 6 | Dosing group | 3 | 0.56 | 0.6497 |
| in LVDs (left | 30 | mg | 5 | Time of | 2 | 2.58 | 0.1053 |
| ventricular end- | | | | measurement | | | |
| systolic internal | 100 | mg | 6 | Dosing group × | 6 | 1.16 | 0.3628 |
| diameter) [%] | | | | time of | | | |
| | | | | measurement | | | |

| | | | | | |
|---|---|---|---|---|---|
| Placebo | 6 | | | | |
| Active | 17 | Dosing group | 1 | 0.77 | 0.3914 |
| group* | | | | | |
| Placebo | 6 | Time of | 2 | 4.54 | 0.0243 |

| | | | |
|---|---|---|---|
| measurement | | | |
| Dosing group × | 2 | 2.33 | 0.1238 |
| time of | | | |
| measurement | | | |

*Combined with 10 mg, 30 mg, and 100 mg dosing groups

4) Changes in left ventricular end-diastolic internal diameter (LVDd) before and after heart attachment

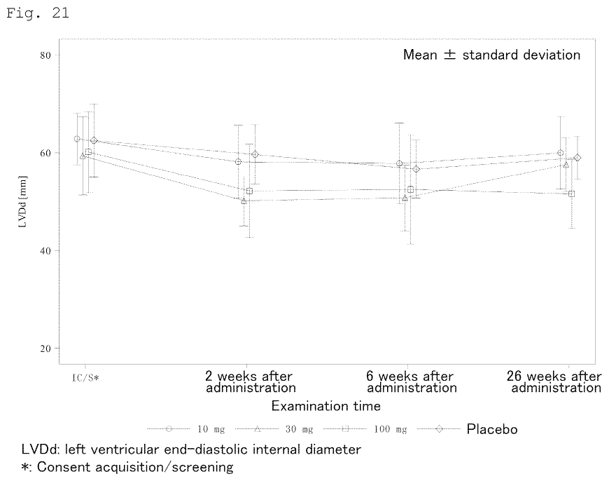

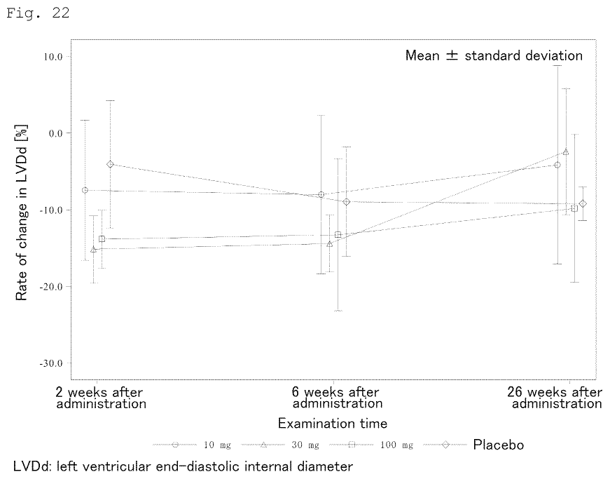

FIG. 21 shows a chart of time-dependent transition of measured LVDd values in echocardiography, FIG. 22 shows a chart of time-dependent transition of the rate of change, Table 24 shows the summary statistics of the measured values, Table 25 shows the summary statistics of the rate of change, and Table 26 shows the results of the analysis of variance of repeated measurements. In addition, a chart of time-dependent transition of the measured LVDd values and the rate of change for each subject, and a list of the measured values and the rate of change were attached in [Appendix 16.2.6].

The rate of change in LVDd (mean±standard deviation) was as follows, in the order of the YS-1402−10 mg group, 30 mg group, 100 mg group, and placebo group:

2 weeks after administration of the investigational drug: −7.46±9.12, −15.14±4.40, −13.84±3.80, −4.06±8.29; 6 weeks: −8.03±10.36, −14.41±3.71, −13.26±9.94, −8.96±7.15%;
26 weeks: −4.14±12.91, −2.40±8.22, −9.82±9.69, −9.21±2.20%.
As a result of analysis of variance of repeated measurements with factors of dose group, time of measurement, and dose group× time of measurement, variations in dose group and dose group×time of measurement were not significant; however, only variation in time of measurement was significant. For the two groups, i.e., the active groups combined with three doses of YS-1402 and the placebo group, variations in dose group and dose group×time of measurement were not significant; however, only variation in time of measurement was significant.

The placebo group showed a decrease over time. As with LDVs, the YS-1402 groups showed a greater decrease than the placebo group at 2 weeks after administration; however, the decrease was not in response to the dose. The decrease was not temporal or dose-dependent at 6 weeks and 26 weeks after administration.

TABLE 24

| | Dosing | | Summary | | 2 weeks after | 6 weeks after | 26 weeks after |
|---|---|---|---|---|---|---|---|
| Test item | group | | statistics | IC/S* | administration | administration | administration |
| LVDd | 10 | mg | Number of subjects | 6 | 6 | 6 | 6 |
| (left ventricular | | | Average value | 62.8 | 58.2 | 57.8 | 60.0 |
| end-diastolic | | | Standard deviation | 5.3 | 7.5 | 8.2 | 7.5 |
| internal diameter) | | | Minimum value | 56 | 47 | 45 | 52 |
| [mm] | | | Median value | 63.5 | 59.5 | 59.0 | 57.5 |
| | | | Maximum value | 69 | 66 | 66 | 73 |
| | 30 | mg | Number of subjects | 5 | 5 | 5 | 5 |
| | | | Average value | 59.4 | 50.2 | 50.8 | 57.6 |
| | | | Standard deviation | 8.0 | 5.1 | 6.7 | 5.5 |
| | | | Minimum value | 52 | 44 | 42 | 52 |
| | | | Median value | 57.0 | 50.0 | 50.0 | 55.0 |
| | | | Maximum value | 71 | 58 | 60 | 66 |
| | 100 | mg | Number of subjects | 6 | 5 | 6 | 5 |
| | | | Average value | 60.2 | 52.2 | 52.5 | 51.6 |
| | | | Standard deviation | 8.3 | 9.5 | 11.1 | 7.0 |
| | | | Minimum value | 50 | 42 | 37 | 42 |
| | | | Median value | 58.5 | 52.0 | 50.5 | 52.0 |
| | | | Maximum value | 75 | 67 | 70 | 60 |

| | | | | | |
|---|---|---|---|---|---|
| Placebo | Number of subjects | 6 | 6 | 6 | 5 |

| | | | | |
|---|---|---|---|---|
| Average value | 62.5 | 59.7 | 56.7 | 59.0 |
| Standard deviation | 7.5 | 6.1 | 6.0 | 4.4 |
| Minimum value | 50 | 50 | 47 | 52 |
| Median value | 65.5 | 61.5 | 59.0 | 59.0 |
| Maximum value | 70 | 65 | 63 | 64 |

*Consent acquisition/screening

TABLE 25

| | Dosing | | 2 weeks after | 6 weeks after | 26 weeks after |
|---|---|---|---|---|---|
| Test item | group | Summary statistics | administration | administration | administration |
| Rate of change | 10 mg | Number of subjects | 6 | 6 | 6 |
| in LVDd (left | | Average value | −7.46 | −8.03 | −4.14 |
| ventricular | | Standard deviation | 9.12 | 10.36 | 12.91 |
| end-diastolic | | Minimum value | −16.1 | −19.6 | −15.9 |
| internal | | Median value | −10.97 | −10.40 | −5.30 |
| diameter) | | Maximum value | 8.2 | 8.2 | 19.7 |
| [%] | 30 mg | Number of subjects | 5 | 5 | 5 |
| | | Average value | −15.14 | −14.41 | −2.40 |
| | | Standard deviation | 4.40 | 3.71 | 8.22 |
| | | Minimum value | −20.3 | −19.2 | −15.5 |
| | | Median value | −15.38 | −15.49 | −1.89 |
| | | Maximum value | −9.4 | −9.4 | 5.8 |
| | 100 mg | Number of subjects | 5 | 6 | 5 |
| | | Average value | −13.84 | −13.26 | −9.82 |
| | | Standard deviation | 3.80 | 9.94 | 9.69 |
| | | Minimum value | −19.3 | −26.0 | −22.6 |
| | | Median value | −12.90 | −12.14 | −8.77 |
| | | Maximum value | −10.3 | 0.0 | 1.7 |
| | Placebo | Number of subjects | 6 | 6 | 5 |
| | | Average value | −4.06 | −8.96 | −9.21 |
| | | Standard deviation | 8.29 | 7.15 | 2.20 |
| | | Minimum value | −12.3 | −17.5 | −11.9 |
| | | Median value | −6.04 | −10.30 | −8.77 |
| | | Maximum value | 10.0 | 4.0 | −6.2 |

TABLE 26

| | | | | Analysis of variance table | | | |
|---|---|---|---|---|---|---|---|
| | | | | | Degrees of | | |
| | Dosing | | Number of | | freedom | F- | p- |
| Test item | group | | subjects | Factor | (numerator) | value | value |
| Rate of change in | 10 | mg | 6 | Dosing group | 3 | 0.67 | 0.5828 |
| LVDd (left ventricular | 30 | mg | 5 | Time of | 2 | 6.21 | 0.0095 |
| end-diastolic | | | | measurement | | | |
| internal diameter) | 100 | mg | 6 | Dosing group × | 6 | 1.96 | 0.1193 |
| [%] | | | | time of | | | |
| | | | | measurement | | | |

| | | | | | |
|---|---|---|---|---|---|
| Placebo | 6 | | | | |
| Active | 17 | Dosing group | 1 | 0.56 | 0.4618 |
| group* | | | | | |
| Placebo | 6 | Time of | 2 | 3.91 | 0.0376 |

| | | | |
|---|---|---|---|
| measurement | | | |
| Dosing group × | 2 | 2.94 | 0.0768 |
| time of | | | |
| measurement | | | |

*Combined with 10 mg, 30 mg, and 100 mg dosing groups

### 5) Changes in Cardiothoracic Ratio (CTR) Before and After Heart Attachment

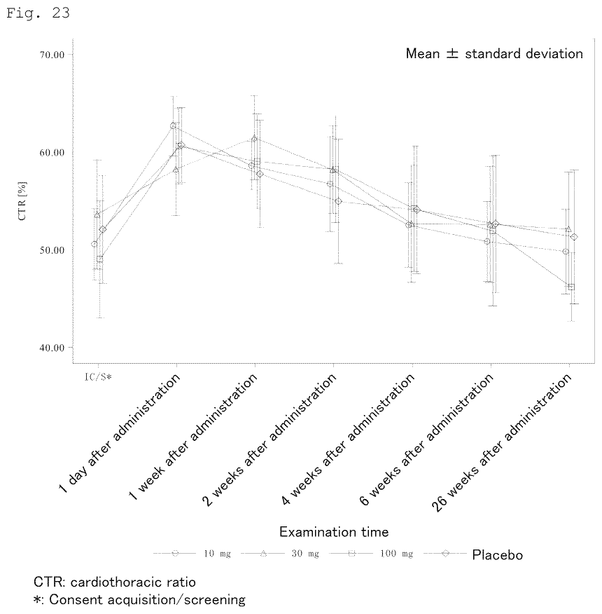

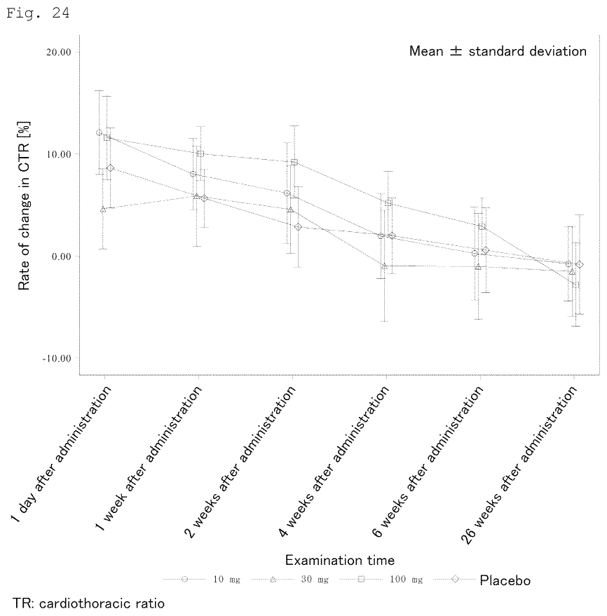

FIG. 23 shows a chart of time-dependent transition of measured CTR values in chest X-ray, FIG. 24 shows a chart of time-dependent transition of the amount of change, Table 27 shows the summary statistics of the measured values, Table 28 shows the summary statistics of the amount of change, and Table 29 shows the results of the analysis of variance of repeated measurements. In addition, a chart of time-dependent transition of the measured CTR values and the rate of change for each subject, and a list of the measured values and the amount of change were attached in [Appendix 16.2.6].

The amount of change in CTR (mean±standard deviation) was as follows, in the order of the YS-1402−10 mg group, 30 mg group, 100 mg group, and placebo group:

1 day after administration of the investigational drug: 12.08±4.10, 4.63±3.92, 11.58±4.09, 8.63±3.93; 2 weeks: 6.17±4.93, 4.58±4.31, 9.22±3.54, 2.87±3.92; 6 weeks: 0.27±4.56, −1.00±5.20, 2.92±2.79, 0.58±4.17′; 26 weeks: −0.75±3.62, −1.48±4.41, −2.80±4.08, −0.82±4.86%.
As a result of analysis of variance of repeated measurements with factors of dose group, time of measurement, and dose group× time of measurement, variations in dose group and dose group×time of measurement were not significant; however, only variation in time of measurement was significant. For the two groups, i.e., the active groups combined with three doses of YS-1402 and the placebo group, variations in dose group and dose group×time of measurement were not significant; however, only variation in time of measurement was significant.

The placebo group showed a maximum value on the first day after administration and then showed a decrease over time. Similarly, the YS-1402 groups showed an approximate maximum value on the first day after administration, and then showed a decrease over time. The maximum values and midway values were not dose-dependent.

TABLE 27

| | | | | | 1 day | 1 week | 2 weeks | 4 weeks | 6 weeks | 26 weeks |
|---|---|---|---|---|---|---|---|---|---|---|
| | Dosing | | Summary | | after admin- | after admin- | after admin- | after admin- | after admin- | after admin- |
| Test item | group | | statistics | IC/S* | istration | istration | istration | istration | istration | istration |
| CTR | 10 | mg | Number of subjects | 6 | 6 | 6 | 6 | 6 | 6 | 6 |
| (cardiothoracic | | | Average value | 50.58 | 62.67 | 58.62 | 56.75 | 52.55 | 50.85 | 49.83 |
| ratio) [%] | | | Standard deviation | 3.66 | 3.08 | 2.47 | 4.85 | 4.31 | 4.09 | 4.33 |
| | | | Minimum value | 45.1 | 59.1 | 55.8 | 50.0 | 47.7 | 46.8 | 44.3 |
| | | | Median value | 50.70 | 63.00 | 58.15 | 56.20 | 51.35 | 49.75 | 50.10 |
| | | | Maximum value | 55.0 | 65.9 | 62.0 | 62.9 | 59.8 | 58.5 | 54.9 |
| | 30 | mg | Number of subjects | 6 | 6 | 5 | 6 | 6 | 6 | 6 |
| | | | Average value | 53.63 | 58.27 | 61.50 | 58.22 | 52.68 | 52.63 | 52.15 |
| | | | Standard deviation | 5.60 | 4.75 | 4.31 | 4.49 | 5.99 | 5.95 | 5.84 |
| | | | Minimum value | 43.7 | 51.2 | 54.9 | 53.2 | 43.6 | 43.1 | 45.3 |
| | | | Median value | 53.75 | 59.55 | 64.30 | 58.25 | 53.85 | 53.50 | 51.35 |
| | | | Maximum value | 59.4 | 64.1 | 64.6 | 64.7 | 58.6 | 60.1 | 59.8 |
| | 100 | mg | Number of subjects | 6 | 6 | 6 | 6 | 6 | 6 | 5 |
| | | | Average value | 49.03 | 60.62 | 59.07 | 58.25 | 54.22 | 51.95 | 46.20 |
| | | | Standard deviation | 6.00 | 3.91 | 4.85 | 5.45 | 6.42 | 7.68 | 3.49 |
| | | | Minimum value | 41.7 | 56.9 | 52.5 | 51.3 | 43.3 | 41.2 | 40.7 |
| | | | Median value | 48.40 | 59.50 | 59.50 | 58.80 | 55.30 | 51.15 | 46.20 |
| | | | Maximum value | 59.7 | 66.7 | 65.3 | 64.8 | 63.1 | 63.7 | 50.2 |

| | | | | | | | | |
|---|---|---|---|---|---|---|---|---|
| Placebo | Number of subjects | 6 | 6 | 6 | 6 | 6 | 6 | 5 |

| | | | | | | | |
|---|---|---|---|---|---|---|---|
| Average value | 52.10 | 60.73 | 57.77 | 54.97 | 54.10 | 52.68 | 51.32 |
| Standard deviation | 5.51 | 3.85 | 5.48 | 6.36 | 6.51 | 7.05 | 6.86 |
| Minimum value | 45.8 | 56.5 | 51.7 | 46.8 | 45.9 | 44.1 | 41.0 |
| Median value | 50.95 | 60.05 | 57.15 | 54.70 | 54.75 | 52.60 | 52.00 |
| Maximum value | 60.5 | 67.4 | 65.7 | 63.8 | 61.6 | 62.1 | 59.9 |

*Consent acquisition/screening

TABLE 28

| | | | | 1 day | 1 week | 2 weeks | 4 weeks | 6 weeks | 26 weeks |
|---|---|---|---|---|---|---|---|---|---|
| | Dosing | | Summary | after admin- | after admin- | after admin- | after admin- | after admin- | after admin- |
| Test item | group | | statistics | istration | istration | istration | istration | istration | istration |
| Amount of change | 10 | mg | Number of subjects | 6 | 6 | 6 | 6 | 6 | 6 |
| in CTR | | | Average value | 12.08 | 8.03 | 6.17 | 1.97 | 0.27 | −0.75 |
| (cardiothoracic | | | Standard deviation | 4.10 | 3.50 | 4.93 | 4.17 | 4.56 | 3.62 |
| ratio) [%] | | | Minimum value | 6.7 | 3.0 | −1.4 | −4.3 | −8.2 | −5.6 |
| | | | Median value | 12.20 | 7.65 | 5.50 | 1.75 | 1.10 | 0.10 |
| | | | Maximum value | 17.8 | 13.0 | 13.5 | 7.0 | 4.6 | 3.8 |
| | 30 | mg | Number of subjects | 6 | 5 | 6 | 6 | 6 | 6 |
| | | | Average value | 4.63 | 5.88 | 4.58 | −0.95 | −1.00 | −1.48 |
| | | | Standard deviation | 3.92 | 4.91 | 4.31 | 5.45 | 5.20 | 4.41 |
| | | | Minimum value | −0.2 | −0.1 | 0.0 | −11.1 | −9.7 | −7.6 |
| | | | Median value | 4.90 | 6.20 | 3.55 | −0.30 | −1.05 | −1.15 |
| | | | Maximum value | 9.5 | 11.5 | 10.1 | 4.0 | 5.5 | 4.6 |
| | 100 | mg | Number of subjects | 6 | 6 | 6 | 6 | 6 | 5 |
| | | | Average value | 11.58 | 10.03 | 9.22 | 5.18 | 2.92 | −2.80 |
| | | | Standard deviation. | 4.09 | 2.63 | 3.54 | 3.13 | 2.79 | 4.08 |
| | | | Minimum value | 7.0 | 5.6 | 5.1 | 1.6 | −0.5 | −9.5 |
| | | | Median value | 11.65 | 10.55 | 9.80 | 5.00 | 2.70 | −1.00 |
| | | | Maximum value | 17.3 | 13.0 | 14.0 | 9.4 | 7.5 | 0.2 |

| | | | | | | | |
|---|---|---|---|---|---|---|---|
| Placebo | Number of subjects | 6 | 6 | 6 | 6 | 6 | 5 |

| | | | | | | |
|---|---|---|---|---|---|---|
| Average value | 8.63 | 5.67 | 2.87 | 2.00 | 0.58 | −0.82 |
| Standard deviation | 3.93 | 2.86 | 3.92 | 3.68 | 4.17 | 4.86 |
| Minimum value | 1.9 | 1.7 | −3.2 | −1.8 | −4.1 | −6.8 |
| Median value | 9.75 | 5.50 | 4.20 | 1.50 | −1.10 | 0.00 |
| Maximum value | 12.2 | 10.3 | 7.3 | 7.2 | 5.9 | 4.1 |

TABLE 29

| | | | | Analysis of variance table | | | |
|---|---|---|---|---|---|---|---|
| | | | | | Degrees of | | |
| | Dosing | | Number of | | freedom | F- | p- |
| Test item | group | | subjects | Factor | (numerator) | value | value |
| Amount of change | 10 | mg | 6 | Dosing group | 3 | 1.86 | 0.1681 |
| in CTR | 30 | mg | 6 | Time of | 5 | 32.98 | p < 0.0001 |
| (cardiothoracic | | | | measurement | | | |
| ratio) [%] | 100 | mg | 6 | Dosing group × | 15 | 1.58 | 0.1529 |
| | | | | time of | | | |
| | | | | measurement | | | |

| | | | | | |
|---|---|---|---|---|---|
| Placebo | 6 | | | | |
| Active | 18 | Dosing group | 1 | 0.44 | 0.5146 |
| group* | | | | | |
| Placebo | 6 | Time of | 5 | 20.18 | p < 0.0001 |
| | | measurement | | | |

| | | | |
|---|---|---|---|
| Dosing group × | 5 | 1.86 | 0.1530 |
| time of | | | |
| measurement | | | |

*Combined with 10 mg, 30 mg, and 100 mg dosing groups

### (4) Changes in Symptoms of Heart Failure Before and After Attachment

1) Changes in NYHA classification before and after heart attachment

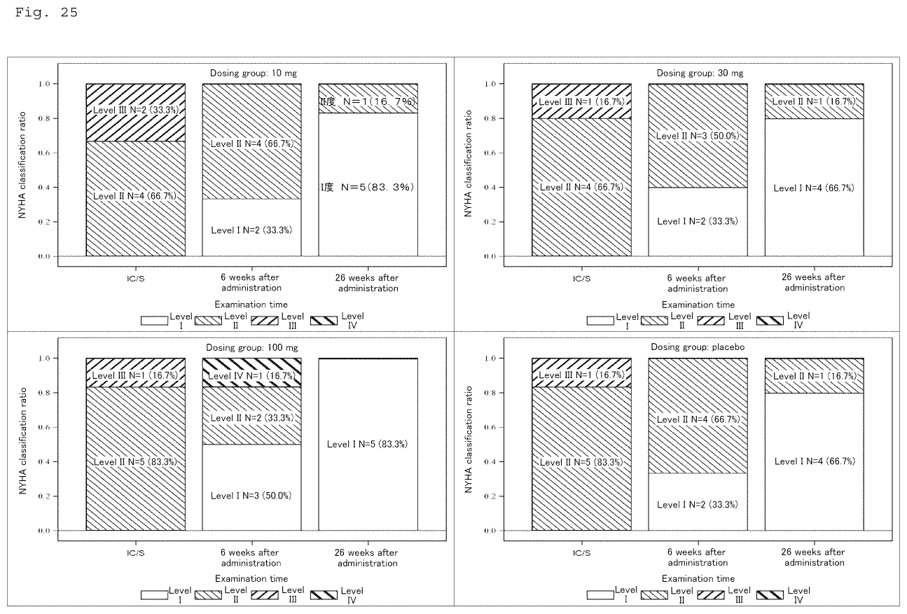

FIG. 25 shows charts of time-dependent transition of the NYHA classification, Table 30 shows a cross-tabulation of the baseline and the NYHA classification at each examination time point, Table 31 shows a dose-response relationship for the rate of improvement in the NYHA classification from the baseline at each examination time point, and Table 32 shows the results of the analysis of variance of repeated measurements. In addition, a list of changes in heart failure symptoms, including measured values of the NYHA classification and the amount of change for each subject, was attached in [Appendix 16.2.6].

The degree of improvement was regarded as the amount of change from the baseline value and defined as follows: improvement of Level II or more, improvement of Level I, constant, and worsening. The distribution of the percentage of the degree of improvement in each YS-1402 dose group was compared to that of the placebo group by applying the Wilcoxon rank sum test; however, there was no significant difference in any YS-1402 dose group from the placebo group. The Cochran-Armitage test was used to evaluate the dose-response relationship for two patterns, i.e., the percentage of improvement of Level II or more and the percentage of improvement of Level I or more; however, none of them was significant. As a result of analysis of variance of repeated measurements with factors of dose group, time of measurement, and dose group×time of measurement, variations in dose group and dose group×time of measurement were not significant; however, only variation in time of measurement was significant.

For the two groups, i.e., the active groups combined with three doses of YS-1402 and the placebo group, variations in dose group and dose group×time of measurement were not significant; however, only variation in time of measurement was significant.

The placebo group showed improvement over time. The YS-1402 groups also showed improvement over time. Particularly in the 100 mg group, all cases were improved to Level I at 26 weeks after administration.

TABLE 30

| | | | 6 weeks after administration | | | | | 26 weeks after administration | | | | |
|---|---|---|---|---|---|---|---|---|---|---|---|---|
| | Dosing | | Level | Level | Level | Level | | Level | Level | Level | Level | |
| Test item | group | IC/S* | I | II | III | IV | Total | I | II | III | IV | Total |
| NYHA | 10 mg | Level I | 0 | 0 | 0 | 0 | 0 | 0 | 0 | 0 | 0 | 0 |
| classification | (N = 6) | Level II | 1 | 3 | 0 | 0 | 4 | 4 | 0 | 0 | 0 | 4 |
| | | Level III | 1 | 1 | 0 | 0 | 2 | 1 | 1 | 0 | 0 | 2 |
| | | Level IV | 0 | 0 | 0 | 0 | 0 | 0 | 0 | 0 | 0 | 0 |
| | | Total | 2 | 4 | 0 | 0 | 6 | 5 | 1 | 0 | 0 | 6 |
| | 30 mg | Level I | 0 | 0 | 0 | 0 | 0 | 0 | 0 | 0 | 0 | 0 |
| | (N = 5) | Level II | 2 | 2 | 0 | 0 | 4 | 3 | 1 | 0 | 0 | 4 |
| | | Level III | 0 | 1 | 0 | 0 | 1 | 1 | 0 | 0 | 0 | 1 |
| | | Level IV | 0 | 0 | 0 | 0 | 0 | 0 | 0 | 0 | 0 | 0 |
| | | Total | 2 | 3 | 0 | 0 | 5 | 4 | 1 | 0 | 0 | 5 |
| | 100 mg | Level I | 0 | 0 | 0 | 0 | 0 | 0 | 0 | 0 | 0 | 0 |
| | (N = 6) | Level II | 3 | 1 | 0 | 1 | 5 | 4 | 0 | 0 | 0 | 4 |
| | | Level III | 0 | 1 | 0 | 0 | 1 | 1 | 0 | 0 | 0 | 1 |
| | | Level IV | 0 | 0 | 0 | 0 | 0 | 0 | 0 | 0 | 0 | 0 |
| | | Total | 3 | 2 | 0 | 1 | 6 | 5 | 0 | 0 | 0 | 5 |
| | Placebo | Level I | 0 | 0 | 0 | 0 | 0 | 0 | 0 | 0 | 0 | 0 |
| | (N = 6) | Level II | 2 | 3 | 0 | 0 | 5 | 4 | 0 | 0 | 0 | 4 |
| | | Level III | 0 | 1 | 0 | 0 | 1 | 0 | 1 | 0 | 0 | 1 |
| | | Level IV | 0 | 0 | 0 | 0 | 0 | 0 | 0 | 0 | 0 | 0 |
| | | Total | 2 | 4 | 0 | 0 | 6 | 4 | 1 | 0 | 0 | 5 |

*Consent acquisition/screening

TABLE 31

| | | 6 weeks after administration | | | | | | | Evaluation of dose-response relationship | | | | |
|---|---|---|---|---|---|---|---|---|---|---|---|---|---|
| | | Improvement | | Improvement | | | | | Improvement | | | Improvement | |
| | Dosing | of Level | | of | | | | p- | of Level | | p- | of Level | p- |
| Test item | group | II or more | | Level I | Constant | Worsening | | value*¹ | II or more | | value*² | I or more | value*² |
| NYHA | Placebo (N = 6) | 0 | (0.0) | 3 (50.0) | 3 (50.0) | 0 | (0.0) | — | 0 | (0.0) | 0.7826 | 3 (50.0) | 0.5308 |
| classification | 10 mg (N = 6) | 1 | (16.7) | 2 (33.3) | 3 (50.0) | 0 | (0.0) | 1.0000 | 1 | (16.7) | | 3 (50.0) | |
| | 30 mg (N = 5) | 0 | (0.0) | 3 (60.0) | 2 (40.0) | 0 | (0.0) | 1.0000 | 0 | (0.0) | | 3 (60.0) | |
| | 100 mg (N = 6) | 0 | (0.0) | 4 (66.7) | 1 (16.7) | 1 | (16.7) | 1.0000 | 0 | (0.0) | | 4 (66.7) | |

| | | 26 weeks after administration | | | | | | | | Evaluation of dose-response relationship | | | | | |
|---|---|---|---|---|---|---|---|---|---|---|---|---|---|---|---|
| | | Improvement | | Improvement | | | | | | Improvement | | | Improvement | | |
| | Dosing | of Level | | of | | | | | p- | of Level | | p- | of Level | | p- |
| Test item | group | II or more | | Level I | | Constant | | Worsening | value*¹ | II or more | | value*² | I or more | | value*² |
| NYHA | Placebo (N = 5) | 0 | (0.0) | 5 | (100.0) | 0 | (0.0) | 0 (0.0) | — | 0 | (0.0) | 0.6466 | 5 | (100.0) | 1.0000 |
| classification | 10 mg (N = 6) | 1 | (16.7) | 5 | (83.3) | 0 | (0.0) | 0 (0.0) | 1.0000 | 1 | (16.7) | | 6 | (100.0) | |
| | 30 mg (N = 5) | 1 | (20.0) | 3 | (60.0) | 1 | (20.0) | 0 (0.0) | 1.0000 | 1 | (20.0) | | 4 | (80.0) | |
| | 100 mg (N = 5) | 1 | (20.0) | 4 | (80.0) | 0 | (0.0) | 0 (0.0) | 1.0000 | 1 | (20.0) | | 5 | (100.0) | |

( ): %
*¹Wilcoxon rank sum test
*²Cochran-Armitage test

TABLE 32

| | | | | Analysis of variance table | | | |
|---|---|---|---|---|---|---|---|
| | | | | | Degrees of | | |
| | Dosing | | Number of | | freedom | F- | p- |
| Test item | group | | subjects | Factor | (numerator) | value | value |
| Rate of change | 10 | mg | 6 | Dosing group | 3 | 0.17 | 0.9158 |
| in NYHA | | | | | | | |
| classification | 30 | mg | 5 | Time of | 1 | 13.68 | 0.0026 |
| | | | | measurement | | | |
| | 100 | mg | 6 | Dosing group × | 3 | 0.19 | 0.8989 |
| | | | | time of | | | |
| | | | | measurement | | | |

| | | | | | |
|---|---|---|---|---|---|
| Placebo | 6 | | | | |
| Active | 17 | Dosing group | 1 | 0.05 | 0.8204 |
| group* | | | | | |
| Placebo | 6 | Time of | 1 | 10.68 | 0.0052 |
| | | measurement | | | |

| | | | |
|---|---|---|---|
| Dosing group × | 1 | 0.06 | 0.8144 |
| time of | | | |
| measurement | | | |

*Combined with 10 mg, 30 mg, and 100 mg dosing groups

### 2) Changes in 6-Minute Walking Distance Before and After Heart Attachment

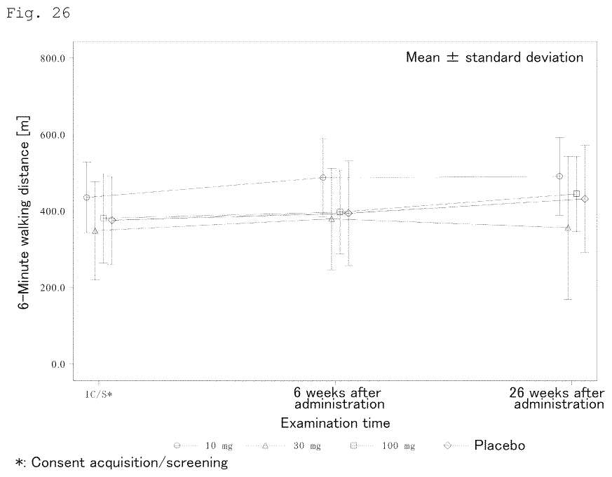

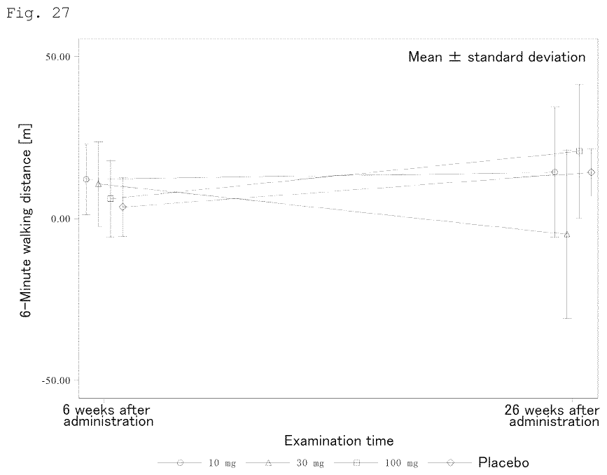

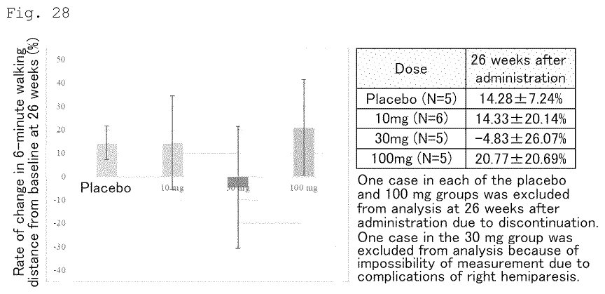

FIG. 26 shows a chart of time-dependent transition of measured values of 6-minute walking distance, FIG. 27 shows a chart of time-dependent transition of the rate of change, Table 33 shows the summary statistics of the measured values, Table 34 shows the summary statistics of the rate of change, Table 35 shows a summary table of the rate of change in 6-minute walking distance at 26 weeks after administration, and Table 36 shows the results of the analysis of variance of repeated measurements. For reference, FIG. 28 shows the rate of change in 6-minute walking distance from the baseline at 26 weeks after administration. In addition, a chart of time-dependent transition of the measured values of 6-minute walking distance and the rate of change for each subject, and a list of changes in heart failure symptoms, including the measured values of 6-minute walking distance and the rate of change, were attached in [Appendix 16.2.6].

The rate of change in 6-minute walking distance (mean±standard deviation) was as follows, in the order of the YS-1402-10 mg group, 30 mg group, 100 mg group, and placebo group:

6 weeks after administration of the investigational drug: 12.07±10.91, 10.67±13.06, 6.07±11.88, 3.57±9.13%; 26 weeks: 14.33±20.14, −4.83±26.07, 20.77±20.69, 14.28±7.24%.
The analysis of variance of repeated measurements with factors of dose group, time of measurement, and dose group×time of measurement did not show any significance. The results were similar for the two groups: the active groups combined with three doses of YS-1402 and the placebo group.

In both the placebo group and the YS-1402 groups, the walking distance generally increased over time. At 6 weeks after administration, the YS-1402 groups showed a greater degree of increase than the placebo group; however, there was no dose-related increase. The 6-minute walking distance at 26 weeks after administration was similar in the 10 mg group to the placebo group and greater in the 100 mg group than in the placebo group, but rather decreased in the 30 mg group. The increase was not dose-related. At 26 weeks after administration, the 100 mg group showed an increase with a rate of change of 6.49% compared to the placebo group.

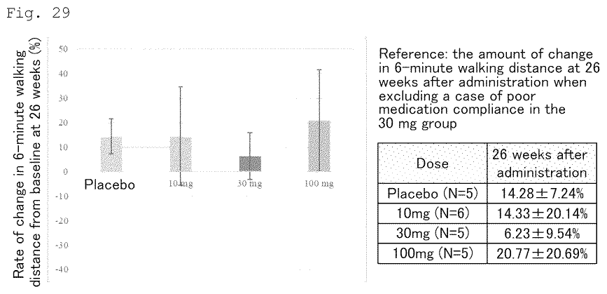

One case (CV-B003) in the YS-1402 30 mg group developed a serious adverse event (congestive heart failure) due to poor medication compliance 26 weeks after administration and 1 week before the test, and the 6-minute walking distance greatly decreased. Therefore, the results excluding this case were shown in FIG. 29 for reference. The rate of change in 6-minute walking distance at 26 weeks after administration was from −4.83±26.07% to 6.23±9.54; however, there was no dose-related increase.

TABLE 33

| | Dosing | | | 6 weeks after | 26 weeks after |
|---|---|---|---|---|---|
| Test item | group | Summary statistics | IC/S* | administration | administration |
| 6-Minute | 10 mg | Number of subjects | 6 | 6 | 6 |
| walking | | Average value | 435.7 | 487.3 | 491.0 |
| distance | | Standard deviation | 92.1 | 102.8 | 101.9 |
| [m] | | Minimum value | 311 | 317 | 339 |
| | | Median value | 428.0 | 500.5 | 506.0 |
| | | Maximum value | 561 | 581 | 598 |
| | 30 mg | Number of subjects | 5 | 5 | 5 |
| | | Average value | 348.4 | 379.2 | 356.0 |
| | | Standard deviation | 128.6 | 132.7 | 187.4 |
| | | Minimum value | 159 | 192 | 81 |
| | | Median value | 329.0 | 410.0 | 349.0 |
| | | Maximum value | 499 | 554 | 593 |
| | 100 mg | Number of subjects | 6 | 5 | 5 |
| | | Average value | 380.8 | 397.4 | 444.8 |
| | | Standard deviation | 116.5 | 109.9 | 99.2 |
| | | Minimum value | 205 | 258 | 280 |
| | | Median value | 403.5 | 474.0 | 485.0 |
| | | Maximum value | 492 | 481 | 534 |
| | Placebo | Number of subjects | 5 | 5 | 5 |
| | | Average value | 375.6 | 394.2 | 431.8 |
| | | Standard deviation | 115.0 | 137.4 | 140.7 |
| | | Minimum value | 200 | 186 | 217 |
| | | Median value | 376.0 | 391.0 | 430.0 |
| | | Maximum value | 512 | 538 | 580 |

*Consent acquisition/screening

TABLE 34

| | Dosing | | 6 weeks after | 26 weeks after |
|---|---|---|---|---|
| Test item | group | Summary statistics | administration | administration |
| Rate of change | 10 mg | Number of subjects | 6 | 6 |
| in 6-Minute | | Average value | 12.07 | 14.33 |
| walking | | Standard deviation | 10.91 | 20.14 |
| distance | | Minimum value | 1.9 | −21.0 |
| [%] | | Median value | 8.75 | 20.95 |
| | | Maximum value | 28.8 | 32.8 |
| | 30 mg | Number of subjects | 5 | 5 |
| | | Average value | 10.67 | −4.83 |
| | | Standard deviation | 13.06 | 26.07 |
| | | Minimum value | −4.0 | −49.1 |
| | | Median value | 11.02 | 2.33 |
| | | Maximum value | 26.2 | 18.8 |
| | 100 mg | Number of subjects | 5 | 5 |
| | | Average value | 6.07 | 20.77 |
| | | Standard deviation | 11.88 | 20.69 |
| | | Minimum value | −3.5 | 0.4 |
| | | Median value | 2.76 | 9.48 |
| | | Maximum value | 25.9 | 48.6 |
| | Placebo | Number of subjects | 5 | 5 |
| | | Average value | 3.57 | 14.28 |
| | | Standard deviation | 9.13 | 7.24 |
| | | Minimum value | −7.0 | 6.4 |
| | | Median value | 5.08 | 13.28 |
| | | Maximum value | 14.8 | 23.1 |

TABLE 35

| | 6-Minute walking distance |
|---|---|
| Placebo group (N = 5)* | 14.28 ± 7.24% |
| 10 mg group (N = 6) | 14.33 ± 20.14% |
| 30 mg group (N = 5) | −4.83 ± 26.07% |
| 100 mg group (N = 5)* | 22.77 ± 20.69% |

*One case in each of the placebo group and 100 mg group was excluded from analysis at 26 weeks after administration due to discontinuation. One case in the 30 mg group was excluded from analysis because of impossibility of measurement due to complications of right hemiparesis.
Rate of change in 6-minute walking distance:
(6-minute walking distance at 26 weeks after administration − 6-minute walking distance at pre-administration)/6-minute walking distance at pre-administration × 100%

TABLE 36

| | | | | Analysis of variance table | | | |
|---|---|---|---|---|---|---|---|
| | | | | | Degrees of | | |
| | Dosing | | Number of | | freedom | F- | p- |
| Test item | group | | subjects | Factor | (numerator) | value | value |
| Rate of change in 6- | 10 | mg | 6 | Dosing group | 3 | 0.81 | 0.5074 |
| Minute walking | 30 | mg | 5 | Time of | 1 | 0.46 | 0.5067 |
| distance [%] | | | | measurement | | | |
| | 100 | mg | 6 | Dosing group × | 3 | 2.14 | 0.1328 |
| | | | | time of | | | |
| | | | | measurement | | | |

| | | | | | |
|---|---|---|---|---|---|
| Placebo | 5 | | | | |
| Active | 17 | Dosing group | 1 | 0.03 | 0.8628 |
| group* | | | | | |
| Placebo | 5 | Time of | 1 | 0.98 | 0.3346 |
| | | measurement | | | |

| | | | |
|---|---|---|---|
| Dosing group × | 1 | 0.78 | 0.3869 |
| time of | | | |
| measurement | | | |

*Combined with 10 mg, 30 mg, and 100 mg dosing groups

### (5) Changes in Myocardial Blood Flow

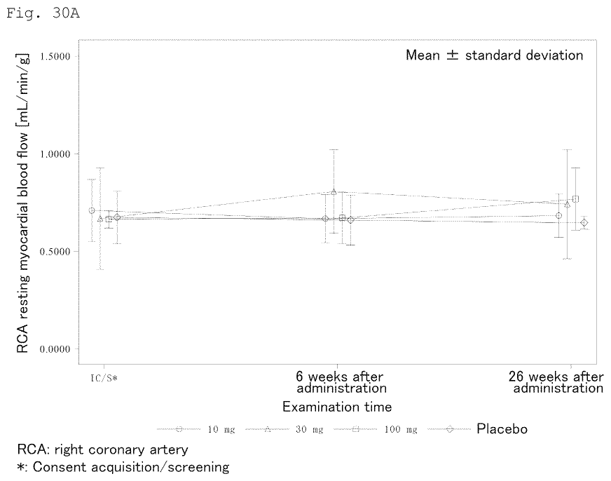

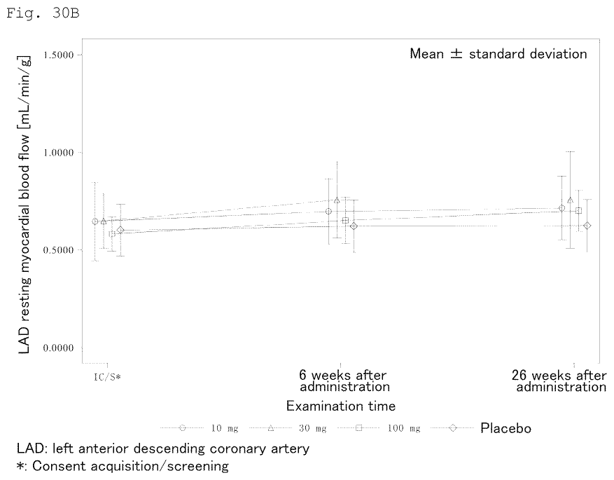

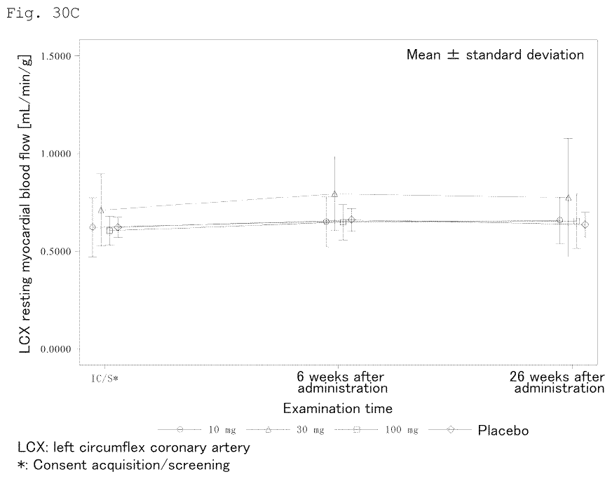

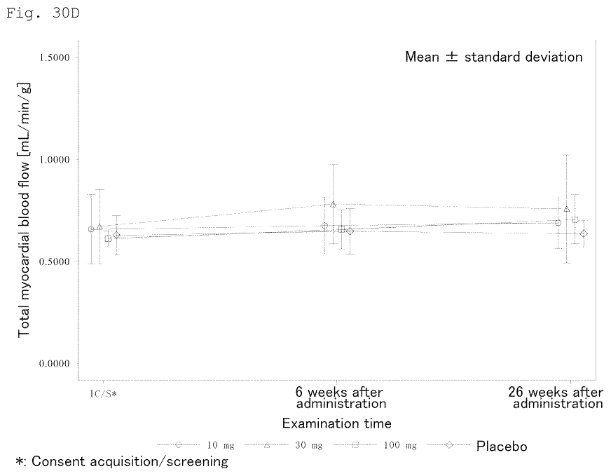

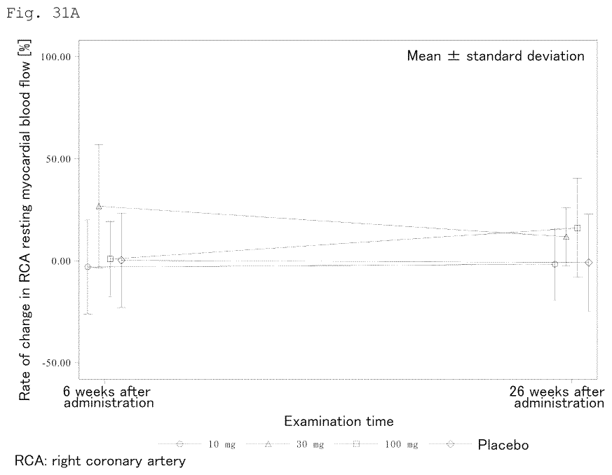

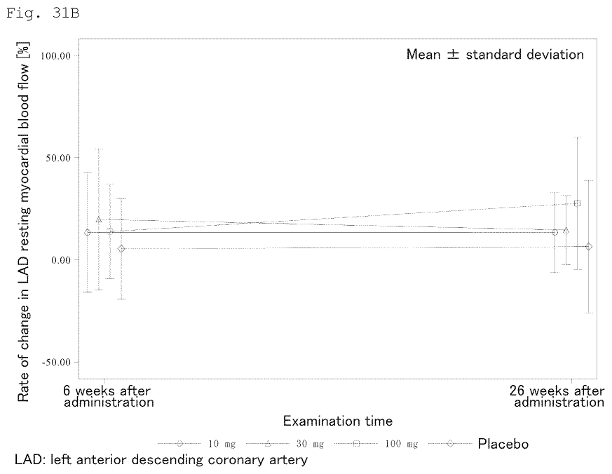

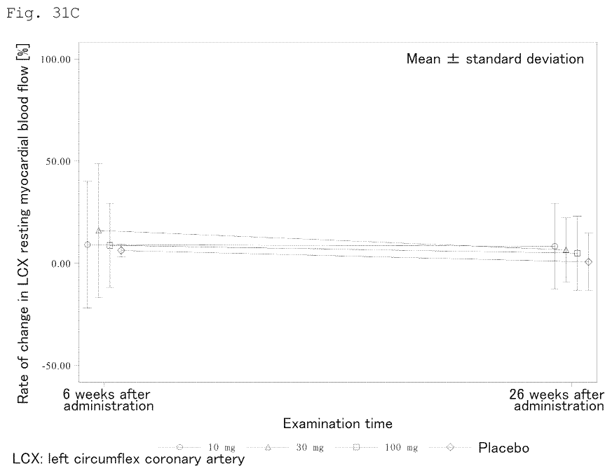

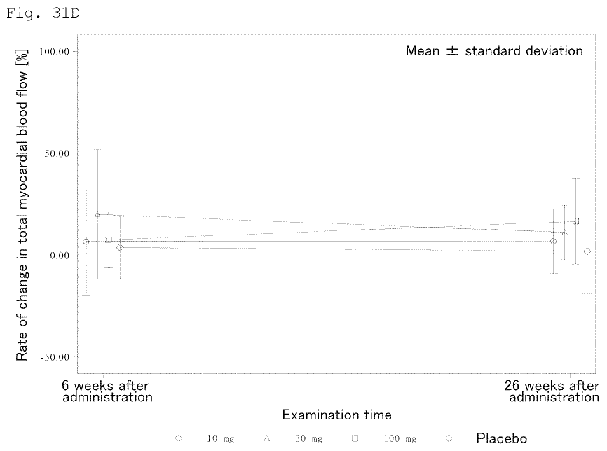

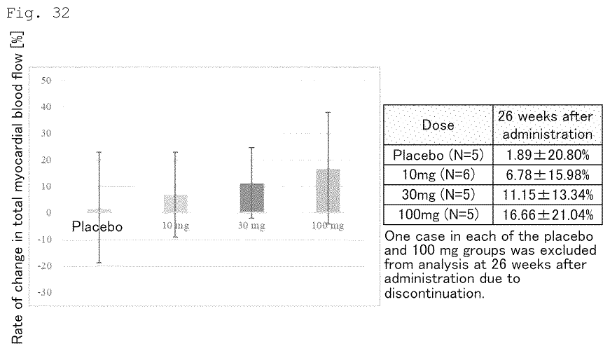

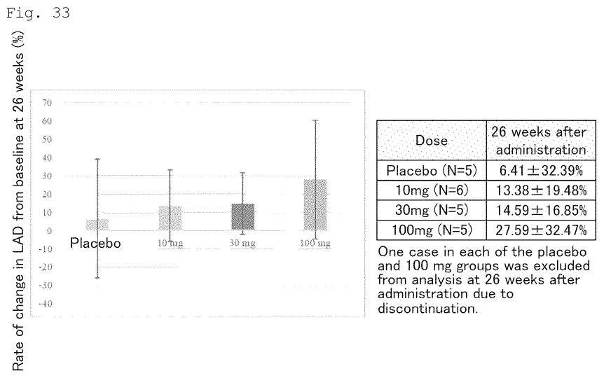

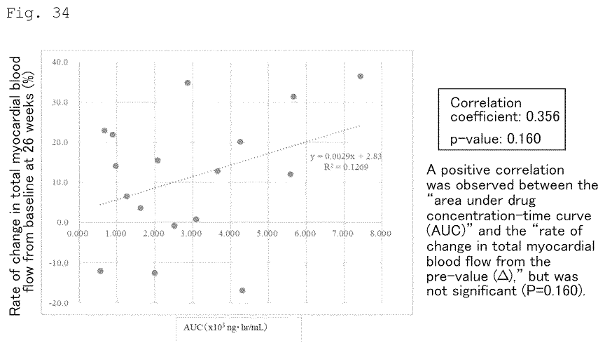

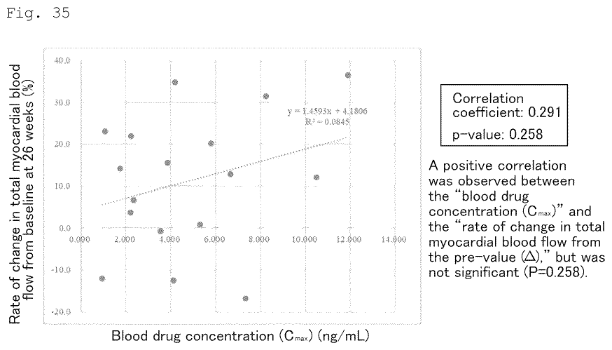

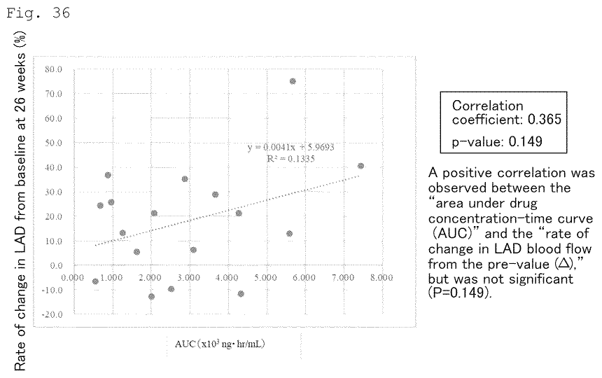

FIG. 30 shows a chart of time-dependent transition of measured values of RCA resting myocardial blood flow, LAD resting myocardial blood flow, LCX resting myocardial blood flow, and total myocardial blood flow by ammonia PET, FIG. 31 shows a chart of time-dependent transition of the rate of change from the baseline, Table 37 shows the summary statistics of the measured values, Table 38 shows the summary statistics of the rate of change from the baseline, and Table 39 shows the results of the analysis of variance of repeated measurements. In addition, Table 40 shows a summary table of the rate of change in myocardial blood flow from the baseline at 26 weeks after administration. For reference, FIG. 32 shows the rate of change in total myocardial blood flow, FIG. 33 shows the rate of change in LAD resting myocardial blood flow, FIGS. 34 and 35 show the correlation between blood concentrations (AUC0-t and Cmax) and the rate of change in total myocardial blood flow from the baseline at 26 weeks after administration of the investigational drug, and FIG. 36 shows the correlation between AUC0-t and the rate of change in LAD resting myocardial blood flow from the baseline at 26 weeks after administration of the investigational drug. In addition, a chart of time-dependent transition of the measured values of the ammonia PET items and the rate of change for each subject, and a list of the measured values and the rate of change [including the name of the site of the left ventricular myocardium (classified into 17 segments) where the investigational drug was attached] were attached in [Appendix 16.2.6]. For reference, blood flow by 17 segments in the left ventricular myocardium for each subject was attached in [Appendix 16.2.9].

The rate of change in RCA resting myocardial blood flow (mean±standard deviation) was as follows, in the order of the YS-1402−10 mg group, 30 mg group, 100 mg group, and placebo group:

6 weeks after administration of the investigational drug: −3.01±23.11, 26.78±30.22, 0.82±18.49, 0.21±23.171; 26 weeks: −1.73±17.50, 11.76±14.33, 16.13±24.20, −0.91±23.85%.
Similarly, the rate of change in LAD resting myocardial blood flow was as follows: 6 weeks after administration of the investigational drug: 13.31±29.16, 19.77±34.62, 13.86±23.11, 5.32±24.69%;
26 weeks: 13.38±19.48, 14.59±16.85, 27.59±32.47, 6.41±32.39%.

The rate of change in LCX resting myocardial blood flow was as follows:

6 weeks after administration of the investigational drug: 9.12±30.95, 16.05±32.75, 8.77±20.53, 6.27±3.11%; 26 weeks: 8.32±20.96, 6.58±15.81, 4.98±18.23, 0.71±14.00%.
The rate of change in total myocardial blood flow was as follows:
6 weeks after administration of the investigational drug: 6.64±26.28, 20.07±31.94, 7.53±13.54, 3.67±15.38%; 26 weeks: 6.78±15.98, 11.15±13.34, 16.66±21.04, 1.89±20.80c.
The analysis of variance of repeated measurements with factors of dose group, time of measurement, and dose group×time of measurement did not show any significance. The results were similar for the two groups: the active groups combined with three doses of YS-1402 and the placebo group.

The RCA resting myocardial blood flow did not change over time in the placebo group. At 6 weeks after administration, the YS-1402 groups generally showed a greater increase in blood flow than the placebo group; however, there was no dose correlation. On the other hand, at 26 weeks after administration, the blood flow increased in a dose-related manner compared to 6 weeks, although it was maintained, decreased, or increased depending on the dosing group.

The LAD resting myocardial blood flow slightly increased over time in the placebo group. The YS-1402 groups showed a greater increase in blood flow than the placebo group at 6 weeks after administration; however, there was no dose correlation. On the other hand, at 26 weeks after administration, the blood flow increased in a dose-related manner compared to 6 weeks, although it was maintained, decreased, or increased depending on the dosing group. The 100 mg group showed an increase in blood flow with a rate of change of 21.18, compared to the placebo group. From the above, at 26 weeks after administration, the LAD myocardial blood flow increased in a dose-related manner in the placebo group, YS-1402−10 mg group, 30 mg group, and 100 mg group. A positive correlation was observed between the blood concentration (AUC0-t) and the rate of change in LAD resting myocardial blood flow from the baseline at 26 weeks after administration of the investigational drug; however, the correlation was not significant (p-value: 0.149).

The LCX resting myocardial blood flow increased 6 weeks after administration in the placebo group. At 26 weeks after administration, the blood flow in the placebo group returned to near the baseline. In the YS-1402 groups, the blood flow increased 6 weeks after administration; however, there was no dose-related increase. At 26 weeks after administration, the increase in blood flow continued from 6 weeks after administration; however, its degree was lowered. The blood flow was higher than the placebo group; however, there was no dose-related increase. From the above, at 26 weeks after administration, compared to the YS-1402 dosing groups, the placebo group showed the lowest LCX myocardial blood flow. On the other hand, no dose-related increase was observed in the YS-1402 dosing groups.

The total myocardial blood flow increased 6 weeks after administration in the placebo group. At 26 weeks after administration, the blood flow in the placebo group returned to near the baseline. The YS-1402 groups outperformed the placebo group at 6 weeks after administration; however, no dose-related increase was observed. On the other hand, at 26 weeks after administration, the blood flow increased in a dose-related manner compared to 6 weeks, although it was maintained, decreased, or increased depending on the dosing group. The 100 mg group showed an increase in blood flow with a rate of change of 14.77 compared to the placebo group. From the above, at 26 weeks after administration, the total myocardial blood flow increased in a dose-related manner in the order of the placebo group, YS-1402−10 mg group, 30 mg group, and 100 mg group. A positive correlation was observed between the blood concentrations (AUC0-t and Cmax) and the rate of change in total myocardial blood flow from the baseline at 26 weeks after administration of the investigational drug; however, the correlation was not significant (p-value, AUC0-t: 0.160, Cmax: 0.258).

TABLE 37

| | Dosing | | | | 6 weeks after | 26 weeks after |
|---|---|---|---|---|---|---|
| Test item | group | | Summary statistics | IC/S* | administration | administration |
| RCA (right | 10 | mg | Number of subjects | 6 | 6 | 6 |
| coronary artery) | | | Average value | 0.7087 | 0.6672 | 0.6830 |
| resting myocardial | | | Standard deviation | 0.1590 | 0.1231 | 0.1124 |
| blood flow | | | Minimum value | 0.561 | 0.450 | 0.518 |
| [mL/min/g] | | | Median value | 0.6890 | 0.7140 | 0.6750 |
| | | | Maximum value | 0.991 | 0.761 | 0.850 |
| | 30 | mg | Number of subjects | 6 | 6 | 6 |
| | | | Average value | 0.6675 | 0.8060 | 0.7415 |
| | | | Standard deviation | 0.2600 | 0.2140 | 0.2802 |
| | | | Minimum value | 0.430 | 0.560 | 0.500 |
| | | | Median value | 0.6120 | 0.7960 | 0.6660 |
| | | | Maximum value | 1.166 | 1.055 | 1.240 |
| | 100 | mg | Number of subjects | 6 | 6 | 5 |
| | | | Average value | 0.6648 | 0.6707 | 0.7672 |
| | | | Standard deviation | 0.0448 | 0.1308 | 0.1592 |
| | | | Minimum value | 0.612 | 0.504 | 0.566 |
| | | | Median value | 0.6575 | 0.7075 | 0.7530 |
| | | | Maximum value | 0.728 | 0.810 | 1.009 |
| | Placebo | | Number of subjects | 6 | 6 | 5 |
| | | | Average value | 0.6740 | 0.6597 | 0.6466 |
| | | | Standard deviation | 0.1343 | 0.1283 | 0.0330 |
| | | | Minimum value | 0.441 | 0.446 | 0.612 |
| | | | Median value | 0.6895 | 0.6750 | 0.6430 |
| | | | Maximum value | 0.847 | 0.794 | 0.692 |
| LAD (left anterior | 10 | mg | Number of subjects | 6 | 6 | 6 |
| descending | | | Average value | 0.6450 | 0.6967 | 0.7135 |
| coronary artery) | | | Standard deviation | 0.2003 | 0.1672 | 0.1623 |
| resting myocardial | | | Minimum value | 0.451 | 0.493 | 0.466 |
| blood flow | | | Median value | 0.5930 | 0.6880 | 0.7765 |
| [mL/min/g] | | | Maximum value | 1.002 | 0.931 | 0.874 |
| | 30 | mg | Number of subjects | 6 | 6 | 6 |
| | | | Average value | 0.6477 | 0.7557 | 0.7555 |
| | | | Standard deviation | 0.1403 | 0.1966 | 0.2474 |
| | | | Minimum value | 0.514 | 0.561 | 0.464 |
| | | | Median value | 0.5910 | 0.7275 | 0.6935 |
| | | | Maximum value | 0.870 | 1.056 | 1.055 |
| | 100 | mg | Number of subjects | 6 | 6 | 5 |
| | | | Average value | 0.5812 | 0.6505 | 0.6998 |
| | | | Standard deviation | 0.0895 | 0.1187 | 0.1047 |
| | | | Minimum value | 0.440 | 0.471 | 0.578 |
| | | | Median value | 0.5980 | 0.6290 | 0.7700 |
| | | | Maximum value | 0.671 | 0.792 | 0.787 |
| | Placebo | | Number of subjects | 6 | 6 | 5 |
| | | | Average value | 0.6015 | 0.6208 | 0.6240 |
| | | | Standard deviation | 0.1329 | 0.1343 | 0.1341 |
| | | | Minimum value | 0.437 | 0.382 | 0.403 |
| | | | Median value | 0.5835 | 0.6450 | 0.6610 |
| | | | Maximum value | 0.823 | 0.786 | 0.754 |
| LCX (left circumflex | 10 | mg | Number of subjects | 6 | 6 | 6 |
| coronary artery) | | | Average value | 0.6220 | 0.6502 | 0.6575 |
| resting myocardial | | | Standard deviation | 0.1512 | 0.1272 | 0.1188 |
| blood flow | | | Minimum value | 0.504 | 0.524 | 0.486 |
| [mL/min/g] | | | Median value | 0.5665 | 0.6260 | 0.6665 |
| | | | Maximum value | 0.888 | 0.814 | 0.795 |
| | 30 | mg | Number of subjects | 6 | 6 | 6 |
| | | | Average value | 0.7110 | 0.7938 | 0.7750 |
| | | | Standard deviation | 0.1839 | 0.1879 | 0.3024 |
| | | | Minimum value | 0.537 | 0.572 | 0.520 |
| | | | Median value | 0.6680 | 0.8085 | 0.6235 |
| | | | Maximum value | 1.001 | 1.001 | 1.215 |
| | 100 | mg | Number of subjects | 6 | 6 | 5 |
| | | | Average value | 0.6042 | 0.6477 | 0.6534 |
| | | | Standard deviation | 0.0728 | 0.0904 | 0.1390 |
| | | | Minimum value | 0.516 | 0.511 | 0.477 |
| | | | Median value | 0.5850 | 0.6455 | 0.6430 |
| | | | Maximum value | 0.701 | 0.768 | 0.866 |
| | Placebo | | Number of subjects | 6 | 6 | 5 |
| | | | Average value | 0.6212 | 0.6602 | 0.6350 |
| | | | Standard deviation | 0.0512 | 0.0588 | 0.0638 |
| | | | Minimum value | 0.557 | 0.596 | 0.591 |
| | | | Median value | 0.6075 | 0.6455 | 0.6120 |
| | | | Maximum value | 0.694 | 0.748 | 0.748 |
| Total myocardial | 10 | mg | Number of subjects | 6 | 6 | 6 |
| blood flow | | | Average value | 0.6575 | 0.6752 | 0.6885 |
| [mL/min/g] | | | Standard deviation | 0.1701 | 0.1376 | 0.1253 |
| | | | Minimum value | 0.500 | 0.490 | 0.487 |
| | | | Median value | 0.5985 | 0.6695 | 0.7050 |
| | | | Maximum value | 0.967 | 0.848 | 0.845 |
| | 30 | mg | Number of subjects | 6 | 6 | 6 |
| | | | Average value | 0.6712 | 0.7805 | 0.7575 |
| | | | Standard deviation | 0.1811 | 0.1957 | 0.2644 |
| | | | Minimum value | 0.496 | 0.564 | 0.492 |
| | | | Median value | 0.6185 | 0.7685 | 0.6615 |
| | | | Maximum value | 0.997 | 1.039 | 1.152 |
| | 100 | mg | Number of subjects | 6 | 6 | 5 |
| | | | Average value | 0.6105 | 0.6567 | 0.7056 |
| | | | Standard deviation | 0.0388 | 0.0953 | 0.1207 |
| | | | Minimum value | 0.558 | 0.528 | 0.546 |
| | | | Median value | 0.6220 | 0.6815 | 0.7330 |
| | | | Maximum value | 0.657 | 0.782 | 0.867 |

| | | | | |
|---|---|---|---|---|
| Placebo | Number of subjects | 6 | 6 | 5 |

| | | | |
|---|---|---|---|
| Average value | 0.6285 | 0.6477 | 0.6354 |
| Standard deviation | 0.0971 | 0.1113 | 0.0669 |
| Minimum value | 0.487 | 0.456 | 0.535 |
| Median value | 0.6320 | 0.6595 | 0.6440 |
| Maximum value | 0.764 | 0.771 | 0.708 |

*Consent acquisition/screening

TABLE 38

| | Dosing | | | 6 weeks after | 26 weeks after |
|---|---|---|---|---|---|
| Test item | group | | Summary statistics | administration | administration |
| Rate of change | 10 | mg | Number of subjects | 6 | 6 |
| in RCA (right | | | Average value | −3.01 | −1.73 |
| coronary artery) | | | Standard deviation | 23.11 | 17.50 |
| resting myocardial | | | Minimum value | −38.7 | −14.2 |
| blood flow | | | Median value | 1.96 | −7.43 |
| [%] | | | Maximum value | 19.6 | 32.3 |
| | 30 | mg | Number of subjects | 6 | 6 |
| | | | Average value | 26.78 | 11.76 |
| | | | Standard deviation | 30.22 | 14.33 |
| | | | Minimum value | −11.3 | −1.2 |
| | | | Median value | 36.74 | 6.63 |
| | | | Maximum value | 55.4 | 38.0 |
| | 100 | mg | Number of subjects | 6 | 5 |
| | | | Average value | 0.82 | 16.13 |
| | | | Standard deviation | 18.49 | 24.20 |
| | | | Minimum value | −24.7 | −22.3 |
| | | | Median value | 3.50 | 23.04 |
| | | | Maximum value | 25.4 | 43.1 |
| | Placebo | | Number of subjects | 6 | 5 |
| | | | Average value | 0.21 | −0.91 |
| | | | Standard deviation | 23.17 | 23.85 |
| | | | Minimum value | −30.4 | −21.4 |
| | | | Median value | −0.65 | −12.31 |
| | | | Maximum value | 36.3 | 38.8 |
| Rate of change | 10 | mg | Number of subjects | 6 | 6 |
| in LAD (left | | | Average value | 13.31 | 13.38 |
| anterior descending | | | Standard deviation | 29.16 | 19.48 |
| coronary artery) | | | Minimum value | −39.9 | −12.8 |
| resting myocardial | | | Median value | 26.90 | 18.64 |
| blood flow | | | Maximum value | 36.3 | 36.8 |
| [%] | 30 | mg | Number of subjects | 6 | 6 |
| | | | Average value | 19.77 | 14.59 |
| | | | Standard deviation | 34.62 | 16.85 |
| | | | Minimum value | −27.1 | −9.7 |
| | | | Median value | 15.51 | 13.81 |
| | | | Maximum value | 76.0 | 35.3 |
| | 100 | mg | Number of subjects | 6 | 5 |
| | | | Average value | 13.86 | 27.59 |
| | | | Standard deviation | 23.11 | 32.47 |
| | | | Minimum value | −29.8 | −11.8 |
| | | | Median value | 18.46 | 21.23 |
| | | | Maximum value | 39.3 | 75.0 |
| | Placebo | | Number of subjects | 6 | 5 |
| | | | Average value | 5.32 | 6.41 |
| | | | Standard deviation | 24.69 | 32.39 |
| | | | Minimum value | −30.7 | −34.6 |
| | | | Median value | 4.68 | 14.42 |
| | | | Maximum value | 45.3 | 39.6 |
| Rate of change in | 10 | mg | Number of subjects | 6 | 6 |
| LCX (left circumflex | | | Average value | 9.12 | 8.32 |
| coronary artery) | | | Standard deviation | 30.95 | 20.96 |
| resting myocardial | | | Minimum value | −34.5 | −20.6 |
| blood flow | | | Median value | 9.82 | 10.52 |
| [%] | | | Maximum value | 50.5 | 35.3 |
| | 30 | mg | Number of subjects | 6 | 6 |
| | | | Average value | 16.05 | 6.58 |
| | | | Standard deviation | 32.75 | 15.81 |
| | | | Minimum value | −32.8 | −11.8 |
| | | | Median value | 12.29 | 1.65 |
| | | | Maximum value | 56.8 | 29.7 |
| | 100 | mg | Number of subjects | 6 | 5 |
| | | | Average value | 8.77 | 4.98 |
| | | | Standard deviation | 20.53 | 18.23 |
| | | | Minimum value | −27.1 | −18.7 |
| | | | Median value | 12.13 | 12.43 |
| | | | Maximum value | 30.8 | 26.8 |
| | Placebo | | Number of subjects | 6 | 5 |
| | | | Average value | 6.27 | 0.71 |
| | | | Standard deviation | 3.11 | 14.00 |
| | | | Minimum value | 0.2 | −11.5 |
| | | | Median value | 7.15 | −0.33 |
| | | | Maximum value | 9.0 | 24.0 |
| Rate of change in | 10 | mg | Number of subjects | 6 | 6 |
| total myocardial | | | Average value | 6.64 | 6.78 |
| blood flow | | | Standard deviation | 26.28 | 15.98 |
| [%] | | | Minimum value | −38.2 | −12.6 |
| | | | Median value | 17.02 | 10.26 |
| | | | Maximum value | 29.8 | 23.0 |
| | 30 | mg | Number of subjects | 6 | 6 |
| | | | Average value | 20.07 | 11.15 |
| | | | Standard deviation | 31.94 | 13.34 |
| | | | Minimum value | −24.7 | −0.8 |
| | | | Median value | 19.88 | 8.23 |
| | | | Maximum value | 64.4 | 34.8 |
| | 100 | mg | Number of subjects | 6 | 5 |
| | | | Average value | 7.53 | 16.66 |
| | | | Standard deviation | 13.54 | 21.04 |
| | | | Minimum value | −15.5 | −16.9 |
| | | | Median value | 10.65 | 20.19 |
| | | | Maximum value | 20.0 | 36.5 |

| | | | |
|---|---|---|---|
| Placebo | Number of subjects | 6 | 5 |

| | | |
|---|---|---|
| Average value | 3.67 | 1.89 |
| Standard deviation | 15.38 | 20.80 |
| Minimum value | −21.1 | −21.1 |
| Median value | 4.08 | 0.59 |
| Maximum value | 27.1 | 25.5 |

TABLE 39

| | | | | Analysis of variance table | | | |
|---|---|---|---|---|---|---|---|
| | | | | | Degrees of | | |
| | Dosing | | Number of | | freedom | F- | p- |
| Test item | group | | subjects | Factor | (numerator) | value | value |
| Rate of change in | 10 | mg | 6 | Dosing group | 3 | 2.10 | 0.1342 |
| RCA (right coronary | 30 | mg | 6 | Time of | 1 | 0.00 | 0.9730 |
| artery) resting | | | | measurement | | | |
| myocardial blood | 100 | mg | 6 | Dosing group × | 3 | 1.00 | 0.4133 |
| flow [%] | | | | time of | | | |
| | | | | measurement | | | |
| | Placebo | | 6 | | | | |
| | Active | | 18 | Dosing group | 1 | 1.05 | 0.3179 |
| | group* | | | | | | |
| | Placebo | | 6 | Time of | 1 | 0.03 | 0.8702 |
| | | | | measurement | | | |
| | | | | Dosing group × | 1 | 0.02 | 0.8956 |
| | | | | time of | | | |
| | | | | measurement | | | |
| Rate of change in | 10 | mg | 6 | Dosing group | 3 | 0.45 | 0.7189 |
| LAD (left anterior | | | | | | | |
| descending coronary | 30 | mg | 6 | Time of | 1 | 0.02 | 0.8959 |
| artery) resting | | | | measurement | | | |
| myocardial blood | 100 | mg | 6 | Dosing group × | 3 | 0.29 | 0.8325 |
| flow [%] | | | | time of | | | |
| | | | | measurement | | | |

| | | | | | |
|---|---|---|---|---|---|
| Placebo | 6 | | | | |
| Active | 18 | Dosing group | 1 | 1.22 | 0.2815 |
| group* | | | | | |
| Placebo | 6 | Time of | 1 | 0.00 | 0.9560 |

| | | | |
|---|---|---|---|
| measurement | | | |
| Dosing group × | 1 | 0.05 | 0.8189 |
| time of | | | |
| measurement | | | |

| | | | | Analysis of variance table | | | |
|---|---|---|---|---|---|---|---|
| | | | | | Degrees of | | |
| | Dosing | | Number of | | freedom | F- | p- |
| Test item | group | | subjects | Factor | (numerator) | value | value |
| Rate of change in LCX | 10 | mg | 6 | Dosing group | 3 | 0.24 | 0.8688 |
| (left circumflex coronary | 30 | mg | 6 | Time of | 1 | 0.67 | 0.4214 |
| artery) resting | | | | measurement | | | |
| myocardial blood | 100 | mg | 6 | Dosing group × | 3 | 0.10 | 0.9595 |
| flow [%] | | | | time of | | | |
| | | | | measurement | | | |
| | Placebo | | 6 | | | | |
| | Active | | 18 | Dosing group | 1 | 0.55 | 0.4660 |
| | group* | | | | | | |
| | Placebo | | 6 | Time of | 1 | 0.58 | 0.4529 |
| | | | | measurement | | | |
| | | | | Dosing group × | 1 | 0.01 | 0.9391 |
| | | | | time of | | | |
| | | | | measurement | | | |
| Rate of change in | 10 | mg | 6 | Dosing group | 3 | 0.82 | 0.5007 |
| total myocardial | 30 | mg | 6 | Time of | 1 | 0.01 | 0.9264 |
| blood flow [%] | | | | measurement | | | |
| | 100 | mg | 6 | Dosing group × | 3 | 0.40 | 0.7555 |
| | | | | time of | | | |
| | | | | measurement | | | |

| | | | | | |
|---|---|---|---|---|---|
| Placebo | 6 | | | | |
| Active | 18 | Dosing group | 1 | 1.42 | 0.2458 |
| group* | | | | | |
| Placebo | 6 | Time of | 1 | 0.04 | 0.8487 |

| | | | |
|---|---|---|---|
| measurement | | | |
| Dosing group × | 1 | 0.02 | 0.8868 |
| time of | | | |
| measurement | | | |

*Combined with 10 mg, 30 mg, and 100 mg dosing groups

TABLE 40

| | | Left anterior | | |
|---|---|---|---|---|
| | | descending artery | Right coronary | Left circumflex |
| | Whole myocardium | (LAD) | artery (RCA) | artery (LCX) |
| Placebo group (N = 5)* | 1.89 ± 20.80% | 6.41 ± 32.39% | −0.91 ± 23.85% | 0.71 ± 14.00% |
| 10 mg group (N = 6) | 6.78 ± 15.98% | 13.38 ± 19.48% | −1.73 ± 17.50% | 8.32 ± 20.96% |
| 30 mg group (N = 6) | 11.15 ± 13.34% | 14.59 ± 16.85% | 11.76 ± 14.33% | 6.58 ± 15.81% |
| 100 mg group (N = 5)* | 16.66 ± 21.04% | 27.59 ± 32.47% | 16.13 ± 24.20% | 4.98 ± 8.23% |

*One case in each of the placebo group and 100 mg group was excluded from analysis at 26 weeks after administration due to discontinuation.
Reasons for discontinuation
Placebo group: Discontinued because the subject underwent mitral valvuloplasty due to exacerbation of mitral regurgitation.
100 mg group: Discontinued because the subject with putaminal hemorrhage stopped coming to the hospital after discharge
Rate of change in myocardial blood flow: (blood flow at 26 weeks after administration − blood flow at pre-administration)/blood flow at pre-administration × 100%

### (6) Changes in Brain Natriuretic Peptide (BNP)

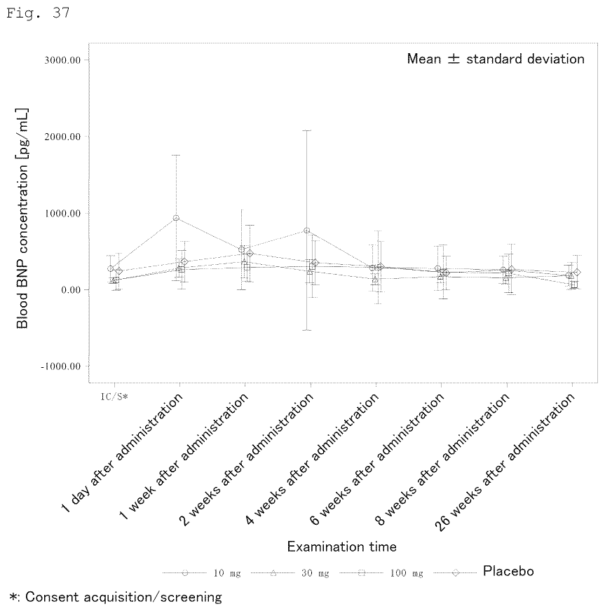

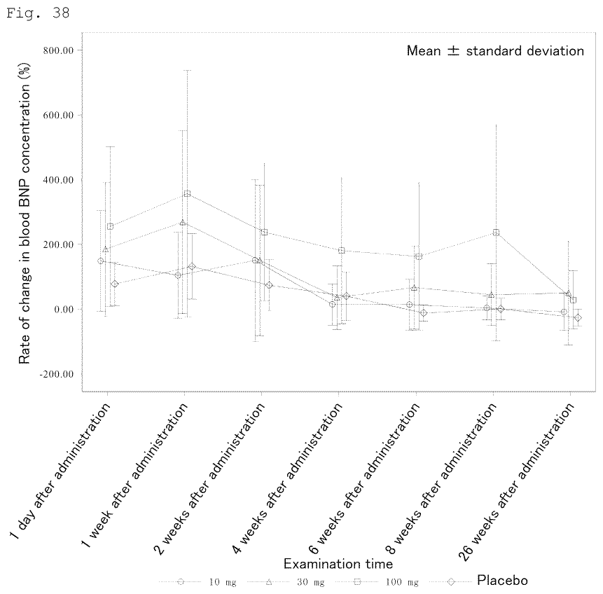

FIG. 37 shows a chart of time-dependent transition of measured blood BNP concentration values, FIG. 38 shows a chart of time-dependent transition of the rate of change, Table 41 shows the summary statistics of the measured values, Table 42 shows the summary statistics of the rate of change, and Table 43 shows the results of the analysis of variance of repeated measurements. In addition, a chart of time-dependent transition of the measured BNP concentration values and the rate of change for each subject, and a list of the measured values and the amount of change were attached in [Appendix 16.2.6].

The rate of change in blood BNP concentration (mean±standard deviation) was as follows, in the order of the YS-1402-10 mg group, 30 mg group, 100 mg group, and placebo group:

1 day after administration of the investigational drug: 148.41±156.07, 184.81±207.24, 255.29±247.65, 77.53±66.69; after 1 week: 104.10±132.51, 268.73±283.06, 356.63±381.21, 131.60±101.04;
after 2 weeks: 150.38±250.68, 150.47±232.24, 237.35±212.48, 73.67±78.76;
after 6 weeks: 13.57±78.60, 66.24±128.44, 162.51±228.08,−12.21±25.15%;
after 26 weeks: −8.84±57.09, 49.44±161.03, 28.00±90.06,−26.84±26.45%.
As a result of analysis of variance of repeated measurements with factors of dose group, time of measurement, and dose group× time of measurement, variations in dose group and dose group×time of measurement were not significant; however, only variation in time of measurement was significant. There was no significance in the active groups combined with three doses of YS-1402 and the placebo group.

The rate of change in blood BNP concentration increased after administration and then decreased in both the placebo group and the YS-1402 groups; however, no definite tendency was observed even at 26 weeks after administration.

TABLE 41

| | | | | | 1 day | 1 week | 2 weeks | 4 weeks | 6 weeks | 8 weeks | 26 weeks |
|---|---|---|---|---|---|---|---|---|---|---|---|
| | | | | | after | after | after | after | after | after | after |
| | Dosing | | Summary | | adminis- | adminis- | adminis- | adminis- | adminis- | adminis- | adminis- |
| Test item | group | | statistics | IC/S* | tration | tration | tration | tration | tration | tration | tration |
| Blood BNP | 10 | mg | Number of subjects | 6 | 5 | 6 | 6 | 6 | 6 | 6 | 6 |
| concentration | | | Average value | 271.60 | 935.90 | 520.80 | 773.52 | 282.15 | 277.13 | 257.00 | 183.88 |
| [pg/mL] | | | Standard deviation | 169.20 | 819.78 | 520.96 | 1304.17 | 302.24 | 289.99 | 180.44 | 136.86 |
| | | | Minimum value | 32.2 | 81.6 | 84.0 | 116.5 | 63.0 | 62.6 | 36.7 | 54.3 |
| | | | Median value | 333.85 | 594.50 | 393.10 | 290.15 | 182.55 | 181.65 | 212.25 | 160.80 |
| | | | Maximum value | 479.5 | 2193.2 | 1471.5 | 3426.5 | 882.9 | 835.2 | 517.3 | 424.5 |
| | 30 | mg | Number of subjects | 6 | 6 | 6 | 6 | 6 | 6 | 6 | 6 |
| | | | Average value | 116.50 | 280.87 | 363.60 | 237.90 | 137.35 | 168.50 | 154.00 | 179.90 |
| | | | Standard deviation | 33.68 | 122.76 | 211.82 | 152.21 | 72.65 | 81.60 | 84.53 | 178.60 |
| | | | Minimum value | 75.5 | 163.0 | 137.0 | 111.5 | 40.2 | 94.3 | 75.6 | 19.9 |
| | | | Median value | 120.30 | 241.05 | 329.90 | 201.90 | 142.10 | 158.50 | 120.30 | 139.25 |
| | | | Maximum value | 152.2 | 478.6 | 640.1 | 512.3 | 232.9 | 315.6 | 288.2 | 511.8 |
| | 100 | mg | Number of subjects | 6 | 6 | 6 | 6 | 6 | 6 | 6 | 5 |
| | | | Average value | 124.50 | 259.98 | 287.55 | 301.53 | 290.62 | 226.48 | 212.10 | 61.24 |
| | | | Standard deviation | 132.63 | 251.86 | 177.77 | 408.76 | 478.11 | 353.54 | 254.02 | 46.05 |
| | | | Minimum value | 10.0 | 81.3 | 116.5 | 56.0 | 50.2 | 20.0 | 88.9 | 19.2 |
| | | | Median value | 68.80 | 193.40 | 201.15 | 161.35 | 96.70 | 105.90 | 105.50 | 50.30 |
| | | | Maximum value | 352.4 | 759.6 | 519.3 | 1127.8 | 1262.9 | 943.7 | 728.5 | 134.2 |

| | | | | | | | | | |
|---|---|---|---|---|---|---|---|---|---|
| Placebo | Number of subjects | 6 | 6 | 6 | 6 | 6 | 6 | 6 | 5 |

| | | | | | | | | |
|---|---|---|---|---|---|---|---|---|
| Average value | 241.23 | 362.73 | 473.18 | 350.38 | 299.57 | 216.07 | 262.50 | 224.40 |
| Standard deviation | 236.05 | 269.22 | 371.52 | 290.90 | 333.75 | 217.36 | 328.69 | 218.47 |
| Minimum value | 33.3 | 69.9 | 99.3 | 106.5 | 94.8 | 26.0 | 44.7 | 50.7 |
| Median value | 186.80 | 321.60 | 366.40 | 268.55 | 182.60 | 144.40 | 130.30 | 122.00 |
| Maximum value | 702.6 | 827.9 | 1122.9 | 884.5 | 968.4 | 627.6 | 914.6 | 595.3 |

*Consent acquisition/screening

TABLE 42

| | | | | 1 day | 1 week | 2 weeks | 4 weeks | 6 weeks | 8 weeks | 26 weeks |
|---|---|---|---|---|---|---|---|---|---|---|
| | | | | after | after | after | after | after | after | after |
| | Dosing | | Summary | adminis- | adminis- | adminis- | adminis- | adminis- | adminis- | adminis- |
| Test item | group | | statistics | tration | tration | tration | tration | tration | tration | tration |
| Rate of change | 10 | mg | Number of subjects | 5 | 6 | 6 | 6 | 6 | 6 | 6 |
| in blood BNP | | | Average value | 148.41 | 104.10 | 150.38 | 14.18 | 13.57 | 3.12 | −8.84 |
| concentration | | | Standard deviation | 156.07 | 132.51 | 250.68 | 63.92 | 78.60 | 36.41 | 57.09 |
| [%] | | | Minimum value | −20.2 | −36.7 | −38.7 | −56.9 | −58.9 | −54.4 | −79.0 |
| | | | Median value | 71.52 | 83.01 | 28.93 | −2.49 | −19.12 | 10.93 | −8.52 |
| | | | Maximum value | 357.4 | 306.2 | 614.6 | 95.7 | 143.2 | 45.5 | 68.6 |
| | 30 | mg | Number of subjects | 6 | 6 | 6 | 6 | 6 | 6 | 6 |
| | | | Average value | 184.81 | 268.73 | 150.47 | 35.41 | 66.24 | 44.37 | 49.44 |
| | | | Standard deviation | 207.24 | 283.06 | 232.24 | 98.07 | 128.44 | 95.31 | 161.03 |
| | | | Minimum value | 21.9 | −8.9 | −25.9 | −73.3 | −35.4 | −38.9 | −75.2 |
| | | | Median value | 70.67 | 198.11 | 56.07 | 22.20 | 25.40 | 3.00 | −0.80 |
| | | | Maximum value | 496.0 | 697.1 | 578.5 | 208.5 | 318.0 | 201.6 | 367.0 |
| | 100 | mg | Number of subjects | 6 | 6 | 6 | 6 | 6 | 6 | 5 |
| | | | Average value | 255.29 | 356.63 | 237.35 | 180.31 | 162.51 | 236.01 | 28.00 |
| | | | Standard deviation | 247.65 | 381.21 | 212.48 | 225.53 | 228.08 | 333.43 | 90.06 |
| | | | Minimum value | −39.3 | 44.0 | −39.6 | −51.6 | −69.2 | −74.8 | −85.7 |
| | | | Median value | 214.96 | 215.48 | 284.55 | 116.45 | 150.75 | 144.72 | 58.63 |
| | | | Maximum value | 713.0 | 1065.0 | 460.0 | 491.5 | 463.0 | 881.0 | 122.1 |

| | | | | | | | | |
|---|---|---|---|---|---|---|---|---|
| Placebo | Number of subjects | 6 | 6 | 6 | 6 | 6 | 6 | 5 |

| | | | | | | | |
|---|---|---|---|---|---|---|---|
| Average value | 77.53 | 131.60 | 73.67 | 39.66 | −12.21 | 0.09 | −26.84 |
| Standard deviation | 66.69 | 101.04 | 78.76 | 75.73 | 25.15 | 33.31 | 26.45 |
| Minimum value | −5.1 | 59.8 | 14.0 | −35.2 | −48.0 | −46.5 | −57.3 |
| Median value | 81.78 | 79.24 | 44.45 | 25.30 | −16.30 | 3.01 | −25.38 |
| Maximum value | 169.7 | 308.9 | 219.8 | 184.7 | 22.8 | 34.2 | 10.0 |

TABLE 43

| | | | Analysis of variance table | | | |
|---|---|---|---|---|---|---|
| | Dosing | Number of | | Degrees of freedom | | |
| Test item | group | subjects | Factor | (numerator) | F-value | p-value |
| Rate of change in | 10 mg | 6 | Dosing group | 3 | 1.95 | 0.1545 |
| blood BNP | 30 mg | 6 | Time of | 6 | 3.27 | 0.0454 |
| concentration [%] | | | measurement | | | |
| | 100 mg | 6 | Dosing group × | 18 | 0.83 | 0.6513 |
| | | | time of | | | |
| | | | measurement | | | |
| | Placebo | 6 | | | | |
| | Active | 18 | Dosing group | 1 | 1.64 | 0.2132 |
| | group* | | | | | |
| | Placebo | 6 | Time of | 6 | 2.68 | 0.0671 |
| | | | measurement | | | |
| | | | Dosing group × | 6 | 0.88 | 0.5371 |
| | | | time of | | | |
| | | | measurement | | | |

*Combined with 10 mg, 30 mg, and 100 mg dosing groups

### (7) Assessment of QOL

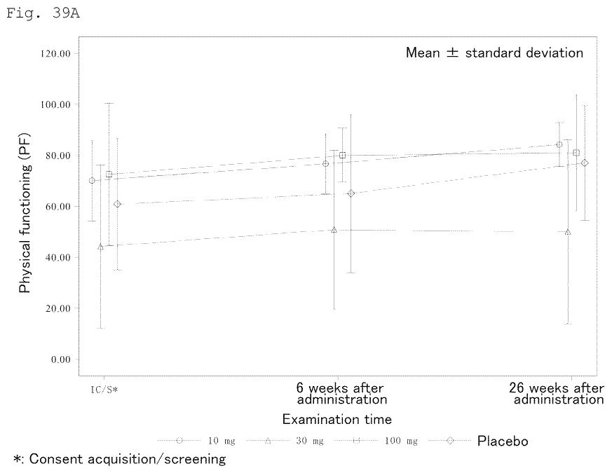

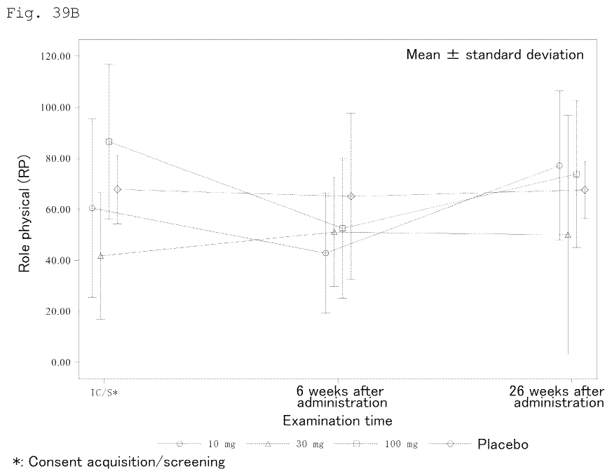

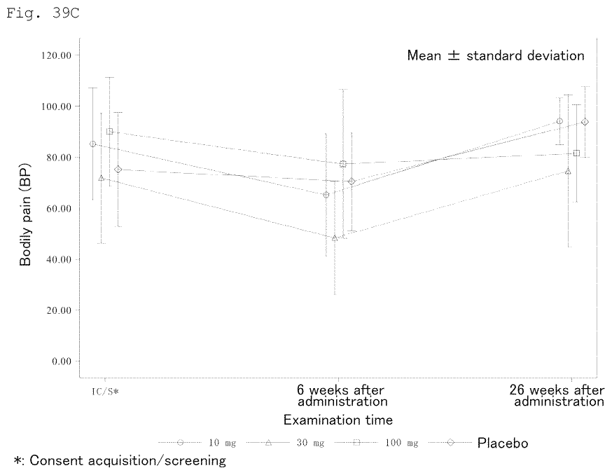

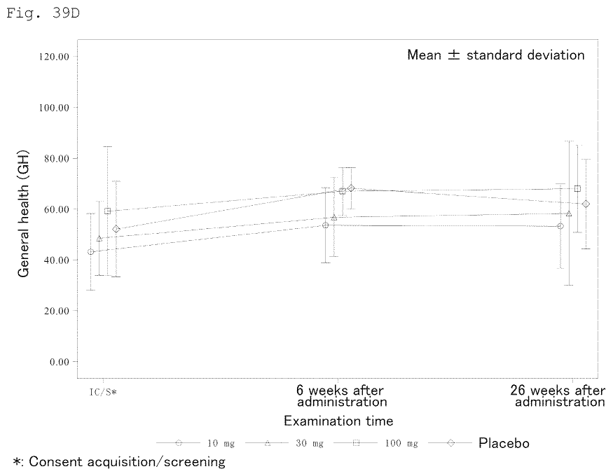

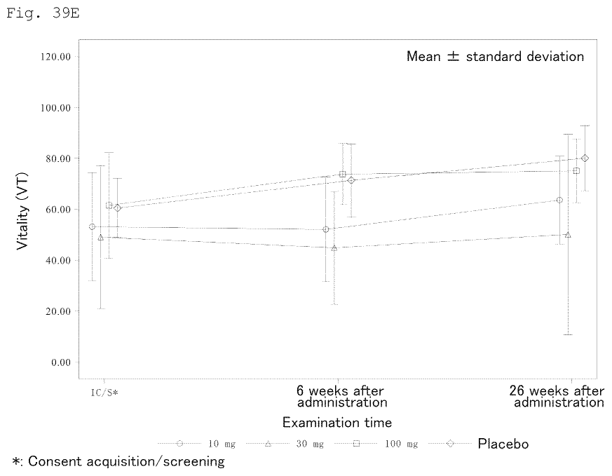

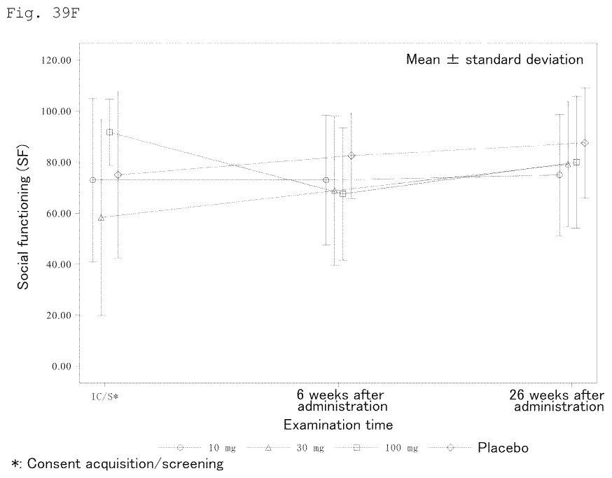

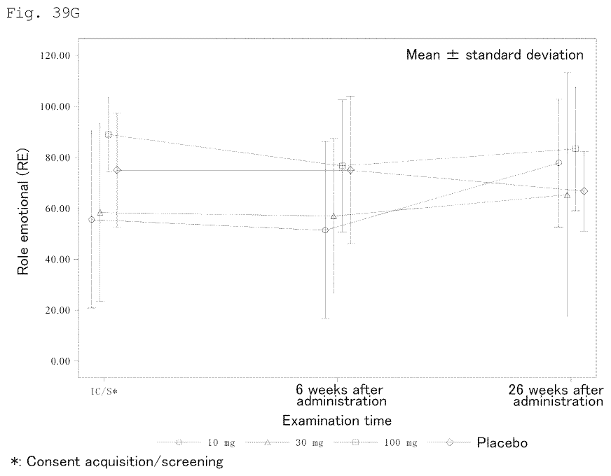

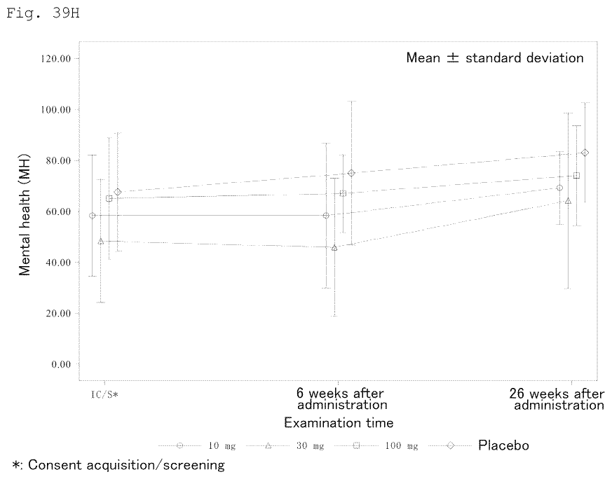

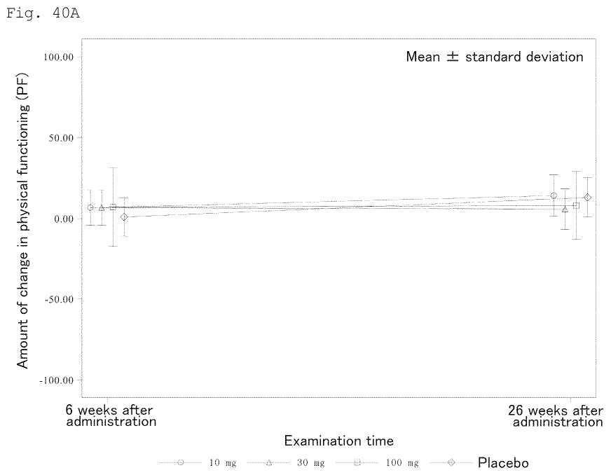

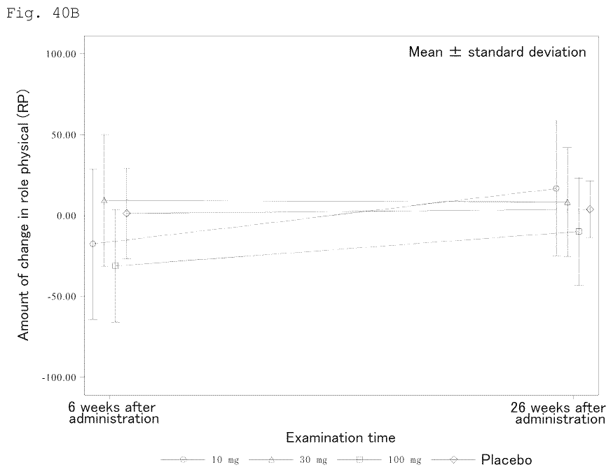

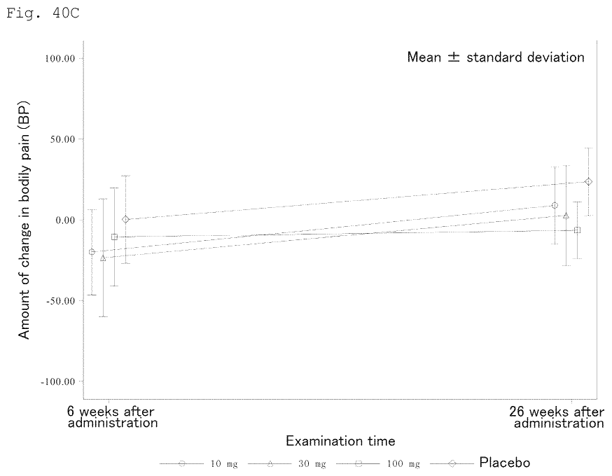

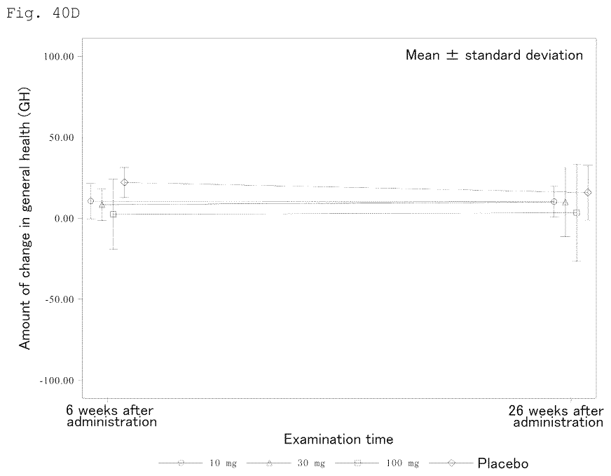

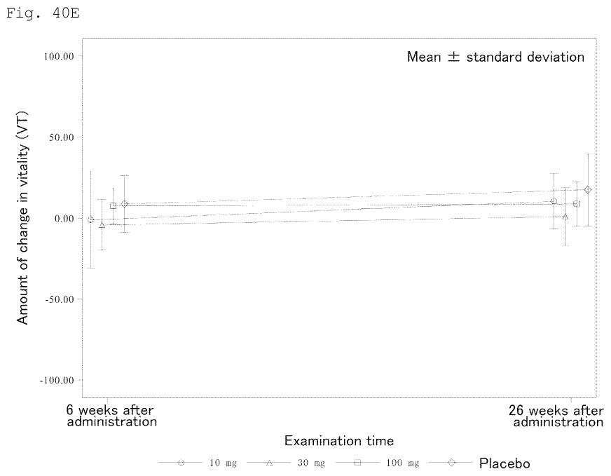

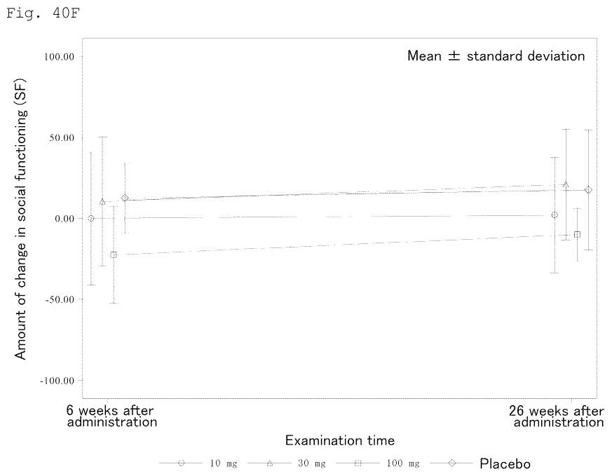

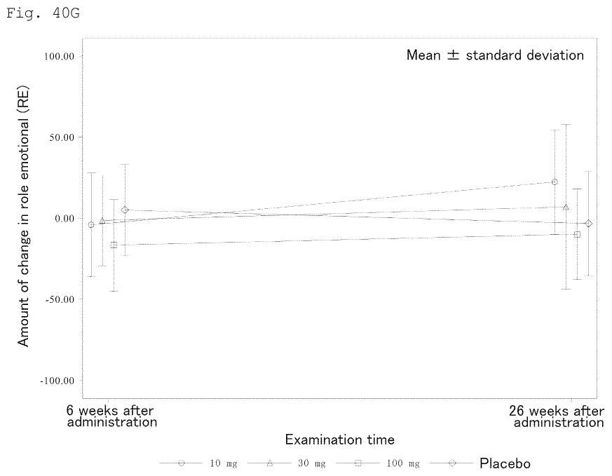

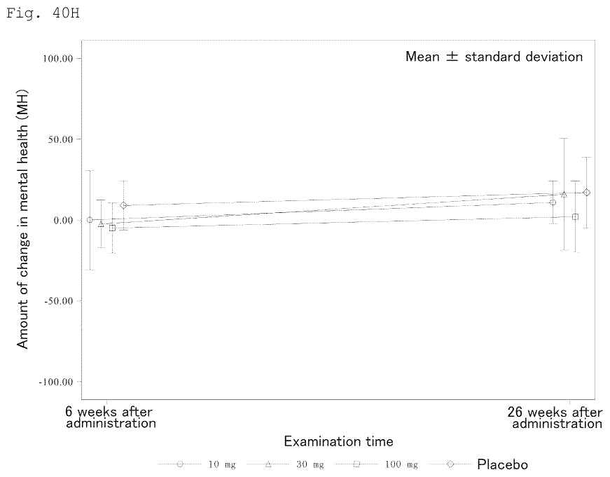

FIG. 39 shows a chart of time-dependent transition of measured score values of 8 subscale items of SF-36 [physical functioning, role physical, bodily pain, general health, vitality, social functioning, role emotional, and mental health] set for QOL assessment, FIG. 40 shows a chart of time-dependent transition of the amount of change, Table 44 shows the summary statistics of the measured values, Table 45 shows the summary statistics of the amount of change, and Table 46 shows the results of the analysis of variance of repeated measurements. In addition, a chart of time-dependent transition of the measured values of SF-36 subscale scores and the amount of change for each subject, and a list of QOL assessment in the measured values and the amount of change were attached in [Appendix 16.2.6].

As a result of analysis of variance, regarding 7 items except bodily pain, the analysis of variance of repeated measurements with factors of dose group, time of measurement, and dose group×time of measurement did not show any significance. The results were similar for the two groups: the active groups combined with three doses of YS-1402 and the placebo group. Regarding bodily pain, as a result of analysis of variance of repeated measurements with factors of dose group, time of measurement, and dose group×time of measurement, variations in dose group and dose group×time of measurement were not significant; however, only variation in time of measurement was significant. For the two groups, i.e., the active groups combined with three doses of YS-1402 and the placebo group, variations in dose group and dose group×time of measurement were not significant; however, only variation in time of measurement was significant.

The placebo group showed improvement in physical functioning, body pain, general health, vitality, social functioning, and mental health at 26 weeks after administration. On the other hand, no improvement tendency was observed in role physical and role emotional.

Physical functioning in the YS-1402 groups was improved 6 weeks after administration compared to the placebo group; however, there was no difference from the placebo group at 26 weeks after administration. Other items did not change compared to the placebo group.

TABLE 44

| | Dosing | Summary | | 6 weeks after | 26 weeks after |
|---|---|---|---|---|---|
| Subscale item | group | statistics | IC/S* | administration | administration |
| Physical functioning | 10 mg | Number of | 6 | 6 | 6 |
| (PF) | | subjects | | | |
| | | Average | 70.00 | 76.67 | 84.17 |
| | | value | | | |
| | | Standard | 15.81 | 11.69 | 8.61 |
| | | deviation | | | |
| | | Minimum | 45.0 | 65.0 | 70.0 |
| | | value | | | |
| | | Median | 72.50 | 75.00 | 85.00 |
| | | value | | | |
| | | Maximum | 90.0 | 90.0 | 95.0 |
| | | value | | | |
| | 30 mg | Number of | 6 | 6 | 6 |
| | | subjects | | | |
| | | Average | 44.17 | 50.83 | 50.00 |
| | | value | | | |
| | | Standard | 32.00 | 31.21 | 36.06 |
| | | deviation | | | |
| | | Minimum | 0.0 | 0.0 | 0.0 |
| | | value | | | |
| | | Median | 45.00 | 52.50 | 50.00 |
| | | value | | | |
| | | Maximum | 90.0 | 95.0 | 95.0 |
| | | value | | | |
| | 100 mg | Number of | 6 | 5 | 5 |
| | | subjects | | | |
| | | Average | 72.50 | 80.00 | 81.00 |
| | | value | | | |
| | | Standard | 27.88 | 10.61 | 22.75 |
| | | deviation | | | |
| | | Minimum | 30.0 | 65.0 | 45.0 |
| | | value | | | |
| | | Median | 80.00 | 80.00 | 85.00 |
| | | value | | | |
| | | Maximum | 100.0 | 95.0 | 100.0 |
| | | value | | | |
| | Placebo | Number of | 6 | 5 | 5 |
| | | subjects | | | |
| | | Average | 60.83 | 65.00 | 77.00 |
| | | value | | | |
| | | Standard | 25.77 | 31.02 | 22.53 |
| | | deviation | | | |
| | | Minimum | 20.0 | 15.0 | 40.0 |
| | | value | | | |
| | | Median | 65.00 | 80.00 | 80.00 |
| | | value | | | |
| | | Maximum | 95.0 | 90.0 | 95.0 |
| | | value | | | |
| Role physical | 10 mg | Number of | 6 | 6 | 6 |
| (RP) | | subjects | | | |
| | | Average | 60.43 | 42.73 | 77.10 |
| | | value | | | |
| | | Standard | 35.07 | 23.54 | 29.22 |
| | | deviation | | | |
| | | Minimum | 25.0 | 0.0 | 31.3 |
| | | value | | | |
| | | Median | 59.40 | 46.90 | 90.65 |
| | | value | | | |
| | | Maximum | 100.0 | 68.8 | 100.0 |
| | | value | | | |
| | 30 mg | Number of | 6 | 6 | 6 |
| | | subjects | | | |
| | | Average | 41.68 | 51.07 | 50.00 |
| | | value | | | |
| | | Standard | 24.90 | 21.42 | 46.77 |
| | | deviation | | | |
| | | Minimum | 0.0 | 31.3 | 0.0 |
| | | value | | | |
| | | Median | 50.05 | 46.90 | 56.25 |
| | | value | | | |
| | | Maximum | 62.5 | 87.5 | 100.0 |
| | | value | | | |
| | 100 mg | Number of | 6 | 5 | 5 |
| | | subjects | | | |
| | | Average | 86.47 | 52.52 | 73.76 |
| | | value | | | |
| | | Standard | 30.21 | 27.44 | 28.78 |
| | | deviation | | | |
| | | Minimum | 25.0 | 313 | 37.5 |
| | | value | | | |
| | | Median | 100.00 | 43.80 | 81.30 |
| | | value | | | |
| | | Maximum | 100.0 | 100.0 | 100.0 |
| | | value | | | |
| | Placebo | Number of | 6 | 5 | 5 |
| | | subjects | | | |
| | | Average | 67.73 | 65.02 | 67.54 |
| | | value | | | |
| | | Standard | 13.35 | 32.66 | 11.20 |
| | | deviation | | | |
| | | Minimum | 50.0 | 12.5 | 50.0 |
| | | value | | | |
| | | Median | 68.80 | 68.80 | 68.80 |
| | | value | | | |
| | | Maximum | 87.5 | 100.0 | 81.3 |
| | | value | | | |
| Bodily pain | 10 mg | Number of | 6 | 6 | 6 |
| (BP) | | subjects | | | |
| | | Average | 85.18 | 65.18 | 94.07 |
| | | value | | | |
| | | Standard | 21.93 | 24.18 | 9.19 |
| | | deviation | | | |
| | | Minimum | 46.7 | 34.4 | 82.2 |
| | | value | | | |
| | | Median | 96.65 | 64.45 | 100.00 |
| | | value | | | |
| | | Maximum | 100.0 | 100.0 | 100.0 |
| | | value | | | |
| | 30 mg | Number of | 6 | 6 | 6 |
| | | subjects | | | |
| | | Average | 71.87 | 48.33 | 74.63 |
| | | value | | | |
| | | Standard | 25.56 | 22.19 | 29.80 |
| | | deviation | | | |
| | | Minimum | 45.6 | 24.4 | 34.4 |
| | | value | | | |
| | | Median | 70.00 | 40.60 | 83.90 |
| | | value | | | |
| | | Maximum | 100.0 | 82.2 | 100.0 |
| | | value | | | |
| | 100 mg | Number of | 6 | 5 | 5 |
| | | subjects | | | |
| | | Average | 90.00 | 77.34 | 81.56 |
| | | value | | | |
| | | Standard | 21.38 | 29.18 | 19.10 |
| | | deviation | | | |
| | | Minimum | 46.7 | 35.6 | 56.7 |
| | | value | | | |
| | | Median | 100.00 | 93.30 | 82.20 |
| | | value | | | |
| | | Maximum | 100.0 | 100.0 | 100.0 |
| | | value | | | |
| | Placebo | Number of | 6 | 5 | 5 |
| | | subjects | | | |
| | | Average | 75.20 | 70.46 | 93.78 |
| | | value | | | |
| | | Standard | 22.30 | 19.19 | 13.91 |
| | | deviation | | | |
| | | Minimum | 45.6 | 56.7 | 68.9 |
| | | value | | | |
| | | Median | 73.90 | 57.80 | 100.00 |
| | | value | | | |
| | | Maximum | 100.0 | 100.0 | 100.0 |
| | | value | | | |
| General health | 10 mg | Number of | 6 | 6 | 6 |
| (GH) | | subjects | | | |
| | | Average | 43.17 | 53.67 | 53.33 |
| | | value | | | |
| | | Standard | 15.12 | 14.72 | 16.51 |
| | | deviation | | | |
| | | Minimum | 27.0 | 32.0 | 32.0 |
| | | value | | | |
| | | Median | 40.00 | 57.00 | 52.00 |
| | | value | | | |
| | | Maximum | 62.0 | 72.0 | 82. |
| | | value | | | |
| | 30 mg | Number of | 6 | 6 | 6 |
| | | subjects | | | |
| | | Average | 48.50 | 56.83 | 58.33 |
| | | value | | | |
| | | Standard | 14.52 | 15.60 | 28.30 |
| | | deviation | | | |
| | | Minimum | 30.0 | 35.0 | 15.0 |
| | | value | | | |
| | | Median | 43.50 | 58.50 | 64.50 |
| | | value | | | |
| | | Maximum | 67.0 | 77.0 | 92.0 |
| | | value | | | |
| | 100 mg | Number of | 6 | 5 | 5 |
| | | subjects | | | |
| | | Average | 59.17 | 67.00 | 68.00 |
| | | value | | | |
| | | Standard | 25.30 | 9.35 | 17.10 |
| | | deviation | | | |
| | | Minimum | 30.0 | 52.0 | 47.0 |
| | | value | | | |
| | | Median | 62.00 | 67.00 | 77.00 |
| | | value | | | |
| | | Maximum | 92.0 | 77.0 | 82.0 |
| | | value | | | |
| | Placebo | Number of | 6 | 5 | 5 |
| | | subjects | | | |
| | | Average | 52.17 | 68.20 | 62.00 |
| | | value | | | |
| | | Standard | 18.72 | 8.07 | 17.68 |
| | | deviation | | | |
| | | Minimum | 30.0 | 57.0 | 47.0 |
| | | value | | | |
| | | Median | 49.50 | 67.00 | 57.00 |
| | | value | | | |
| | | Maximum | 82.0 | 77.0 | 92.0 |
| | | value | | | |
| Vitality | 10 mg | Number of | 6 | 6 | 6 |
| (VT) | | subjects | | | |
| | | Average | 53.15 | 52.10 | 63.57 |
| | | value | | | |
| | | Standard | 21.21 | 20.42 | 17.41 |
| | | deviation | | | |
| | | Minimum | 25.0 | 12.5 | 31.3 |
| | | value | | | |
| | | Median | 53.15 | 59.40 | 65.65 |
| | | value | | | |
| | | Maximum | 81.3 | 68.8 | 81.3 |
| | | value | | | |
| | 30 mg | Number of | 6 | 6 | 6 |
| | | subjects | | | |
| | | Average | 49.00 | 44.82 | 50.05 |
| | | value | | | |
| | | Standard | 28.06 | 22.15 | 39.33 |
| | | deviation | | | |
| | | Minimum | 6.3 | 18.8 | 6.3 |
| | | value | | | |
| | | Median | 50.05 | 46.90 | 50.05 |
| | | value | | | |
| | | Maximum | 81.3 | 68.8 | 93.8 |
| | | value | | | |
| | 100 mg | Number of | 6 | 5 | 5 |
| | | subjects | | | |
| | | Average | 61.48 | 73.78 | 75.04 |
| | | value | | | |
| | | Standard | 20.71 | 12.01 | 12.49 |
| | | deviation | | | |
| | | Minimum | 37.5 | 56.3 | 56.3 |
| | | value | | | |
| | | Median | 65.65 | 75.00 | 81.30 |
| | | value | | | |
| | | Maximum | 81.3 | 87.5 | 87.5 |
| | | value | | | |
| | Placebo | Number of | 6 | 5 | 5 |
| | | subjects | | | |
| | | Average | 60.43 | 71.28 | 80.04 |
| | | value | | | |
| | | Standard | 11.63 | 14.39 | 12.81 |
| | | deviation | | | |
| | | Minimum | 50.0 | 56.3 | 68.8 |
| | | value | | | |
| | | Median | 56.30 | 68.80 | 75.00 |
| | | value | | | |
| | | Maximum | 75.0 | 93.8 | 93.8 |
| | | value | | | |
| Social functioning | 10 mg | Number of | 6 | 6 | 6 |
| (SF) | | subjects | | | |
| | | Average | 72.92 | 72.92 | 75.00 |
| | | value | | | |
| | | Standard | 32.03 | 25.52 | 23.72 |
| | | deviation | | | |
| | | Minimum | 12.5 | 37.5 | 50.0 |
| | | value | | | |
| | | Median | 75.00 | 75.00 | 75.00 |
| | | value | | | |
| | | Maximum | 100.0 | 100.0 | 100.0 |
| | | value | | | |
| | 30 mg | Number of | 6 | 6 | 6 |
| | | subjects | | | |
| | | Average | 58.33 | 68.75 | 79.17 |
| | | value | | | |
| | | Standard | 38.46 | 29.32 | 24.58 |
| | | deviation | | | |
| | | Minimum | 0.0 | 25.0 | 50.0 |
| | | value | | | |
| | | Median | 68.75 | 68.75 | 87.50 |
| | | value | | | |
| | | Maximum | 100.0 | 100.0 | 100.0 |
| | | value | | | |
| | 100 mg | Number of | 6 | 5 | 5 |
| | | subjects | | | |
| | | Average | 91.67 | 67.50 | 80.00 |
| | | value | | | |
| | | Standard | 12.91 | 25.92 | 25.92 |
| | | deviation | | | |
| | | Minimum | 75.0 | 37.5 | 37.5 |
| | | value | | | |
| | | Median | 100.00 | 62.50 | 87.50 |
| | | value | | | |
| | | Maximum | 100.0 | 100.0 | 100.0 |
| | | value | | | |
| | Placebo | Number of | 6 | 5 | 5 |
| | | subjects | | | |
| | | Average | 75.00 | 82.50 | 87.50 |
| | | value | | | |
| | | Standard | 32.60 | 16.77 | 21.65 |
| | | deviation | | | |
| | | Minimum | 12.5 | 62.5 | 50.0 |
| | | value | | | |
| | | Median | 81.25 | 75.00 | 100.00 |
| | | value | | | |
| | | Maximum | 100.0 | 100.0 | 100.0 |
| | | value | | | |
| Role emotional | 10 mg | Number of | 6 | 6 | 6 |
| (RE) | | subjects | | | |
| | | Average | 55.55 | 51.38 | 77.78 |
| | | value | | | |
| | | Standard | 34.82 | 34.73 | 25.09 |
| | | deviation | | | |
| | | Minimum | 16.7 | 0.0 | 50.0 |
| | | value | | | |
| | | Median | 54.15 | 50.00 | 83.35 |
| | | value | | | |
| | | Maximum | 100.0 | 100.0 | 100.0 |
| | | value | | | |
| | 30 mg | Number of | 6 | 6 | 6 |
| | | subjects | | | |
| | | Average | 58.33 | 56.95 | 65.27 |
| | | value | | | |
| | | Standard | 34.95 | 30.46 | 47.85 |
| | | deviation | | | |
| | | Minimum | 16.7 | 16.7 | 0.0 |
| | | value | | | |
| | | Median | 50.00 | 58.35 | 91.65 |
| | | value | | | |
| | | Maximum | 100.0 | 91.7 | 100.0 |
| | | value | | | |
| | 100 mg | Number of | 6 | 5 | 5 |
| | | subjects | | | |
| | | Average | 88.90 | 76.66 | 83.34 |
| | | value | | | |
| | | Standard | 14.58 | 25.95 | 24.28 |
| | | deviation | | | |
| | | Minimum | 66.7 | 41.7 | 41.7 |
| | | value | | | |
| | | Median | 95.85 | 83.30 | 91.70 |
| | | value | | | |
| | | Maximum | 100.0 | 100.0 | 100.0 |
| | | value | | | |
| | Placebo | Number of | 6 | 5 | 5 |
| | | subjects | | | |
| | | Average | 74.98 | 74.98 | 66.68 |
| | | value | | | |
| | | Standard | 22.37 | 28.86 | 15.61 |
| | | deviation | | | |
| | | Minimum | 50.0 | 25.0 | 50.0 |
| | | value | | | |
| | | Median | 70.80 | 83.30 | 66.70 |
| | | value | | | |
| | | Maximum | 100.0 | 100.0 | 91.7 |
| | | value | | | |
| Mental health | 10 mg | Number of | 6 | 6 | 6 |
| (MH) | | subjects | | | |
| | | Average | 58.33 | 58.33 | 69.17 |
| | | value | | | |
| | | Standard | 23.80 | 28.40 | 14.29 |
| | | deviation | | | |
| | | Minimum | 30.0 | 5.0 | 50.0 |
| | | value | | | |
| | | Median | 55.00 | 67.50 | 67.50 |
| | | value | | | |
| | | Maximum | 90.0 | 80.0 | 90.0 |
| | | value | | | |
| | 30 mg | Number of | 6 | 6 | 6 |
| | | subjects | | | |
| | | Average | 48.33 | 45.83 | 64.17 |
| | | value | | | |
| | | Standard | 24.22 | 27.10 | 34.41 |
| | | deviation | | | |
| | | Minimum | 20.0 | 5.0 | 10.0 |
| | | value | | | |
| | | Median | 52.50 | 42.50 | 72.50 |
| | | value | | | |
| | | Maximum | 75.0 | 80.0 | 100.0 |
| | | value | | | |
| | 100 mg | Number of | 6 | 5 | 5 |
| | | subjects | | | |
| | | Average | 65.00 | 67.00 | 74.00 |
| | | value | | | |
| | | Standard | 23.87 | 15.25 | 19.49 |
| | | deviation | | | |
| | | Minimum | 30.0 | 40.0 | 40.0 |
| | | value | | | |
| | | Median | 65.00 | 75.00 | 85.00 |
| | | value | | | |
| | | Maximum | 100.0 | 75.0 | 85.0 |
| | | value | | | |
| | Placebo | Number of | 6 | 5 | 5 |
| | | subjects | | | |
| | | Average | 67.50 | 75.00 | 83.00 |
| | | value | | | |
| | | Standard | 23.18 | 28.28 | 19.56 |
| | | deviation | | | |
| | | Minimum | 25.0 | 40.0 | 55.0 |
| | | value | | | |
| | | Median | 72.50 | 85.00 | 95.00 |
| | | value | | | |
| | | Maximum | 95.0 | 100.0 | 100.0 |
| | | value | | | |

*Consent acquisition/screening

TABLE 45

| | Dosing | Summary | 6 weeks after | 26 weeks after |
|---|---|---|---|---|
| Subscale item | group | statistics | administration | administration |
| Amount of change in | 10 mg | Number of | 6 | 6 |
| physical functioning (PF) | | subjects | | |
| | | Average value | 6.67 | 14.17 |
| | | Standard | 10.80 | 12.81 |
| | | deviation | | |
| | | Minimum value | −10.0 | −5.0 |
| | | Median value | 7.50 | 17.50 |
| | | Maximum | 20.0 | 25.0 |
| | | value | | |
| | 30 mg | Number of | 6 | 6 |
| | | subjects | | |
| | | Average value | 6.67 | 5.83 |
| | | Standard | 10.80 | 12.42 |
| | | deviation | | |
| | | Minimum value | −10.0 | −5.0 |
| | | Median value | 7.50 | 2.50 |
| | | Maximum | 20.0 | 30.0 |
| | | value | | |
| | 100 mg | Number of | 5 | 5 |
| | | subjects | | |
| | | Average value | 7.00 | 8.00 |
| | | Standard | 24.39 | 21.10 |
| | | deviation | | |
| | | Minimum value | −20.0 | −5.0 |
| | | Median value | 0.00 | 0.00 |
| | | Maximum | 35.0 | 45.0 |
| | | value | | |
| | Placebo | Number of | 5 | 5 |
| | | subjects | | |
| | | Average value | 1.00 | 13.00 |
| | | Standard | 11.94 | 12.04 |
| | | deviation | | |
| | | Minimum value | −10.0 | 0.0 |
| | | Median value | −5.00 | 10.00 |
| | | Maximum | 20.0 | 30.0 |
| | | value | | |
| Amount of change in role | 10 mg | Number of | 6 | 6 |
| physical (RP) | | subjects | | |
| | | Average value | −17.70 | 16.67 |
| | | Standard | 46.69 | 41.79 |
| | | deviation | | |
| | | Minimum value | −81.3 | −50.0 |
| | | Median value | −15.60 | 15.60 |
| | | Maximum | 31.3 | 68.8 |
| | | value | | |
| | 30 mg | Number of | 6 | 6 |
| | | subjects | | |
| | | Average value | 9.38 | 8.32 |
| | | Standard | 40.83 | 33.94 |
| | | deviation | | |
| | | Minimum value | −31.2 | −43.8 |
| | | Median value | 3.10 | 25.00 |
| | | Maximum | 87.5 | 37.5 |
| | | value | | |
| | 100 mg | Number of | 5 | 5 |
| | | subjects | | |
| | | Average value | −31.24 | −10.00 |
| | | Standard | 34.79 | 33.24 |
| | | deviation | | |
| | | Minimum value | −62.5 | −62.5 |
| | | Median value | −50.00 | 0.00 |
| | | Maximum | 12.5 | 25.0 |
| | | value | | |
| | Placebo | Number of | 5 | 5 |
| | | subjects | | |
| | | Average value | 1.24 | 3.76 |
| | | Standard | 28.08 | 17.47 |
| | | deviation | | |
| | | Minimum value | −37.5 | −25.0 |
| | | Median value | 0.00 | 12.50 |
| | | Maximum | 31.2 | 18.8 |
| | | value | | |
| Amount of change in | 10 mg | Number of | 6 | 6 |
| bodily pain (BP) | | subjects | | |
| | | Average value | −20.00 | 8.88 |
| | | Standard | 26.44 | 23.88 |
| | | deviation | | |
| | | Minimum value | −54.4 | −17.8 |
| | | Median value | −20.60 | 3.35 |
| | | Maximum | 11.1 | 53.3 |
| | | value | | |
| | 30 mg | Number of | 6 | 6 |
| | | subjects | | |
| | | Average value | −23.53 | 2.77 |
| | | Standard | 36.45 | 31.03 |
| | | deviation | | |
| | | Minimum value | −65.6 | −32.2 |
| | | Median value | −32.80 | 0.00 |
| | | Maximum | 36.6 | 54.4 |
| | | value | | |
| | 100 mg | Number of | 5 | 5 |
| | | subjects | | |
| | | Average value | −10.66 | −6.44 |
| | | Standard | 30.42 | 17.48 |
| | | deviation | | |
| | | Minimum value | −64.4 | −31.1 |
| | | Median value | 0.00 | 0.00 |
| | | Maximum | 11.1 | 10.0 |
| | | value | | |
| | Placebo | Number of | 5 | 5 |
| | | subjects | | |
| | | Average value | 0.22 | 23.54 |
| | | Standard | 27.11 | 20.91 |
| | | deviation | | |
| | | Minimum value | −42.2 | 0.0 |
| | | Median value | 0.00 | 20.00 |
| | | Maximum | 32.2 | 54.4 |
| | | value | | |
| Amount of change in | 10 mg | Number of | 6 | 6 |
| general health (GH) | | subjects | | |
| | | Average value | 10.50 | 10.17 |
| | | Standard | 10.86 | 9.45 |
| | | deviation | | |
| | | Minimum value | 2.0 | −3.0 |
| | | Median value | 7.00 | 8.50 |
| | | Maximum | 32.0 | 22.0 |
| | | value | | |
| | 30 mg | Number of | 6 | 6 |
| | | subjects | | |
| | | Average value | 8.33 | 9.83 |
| | | Standard | 9.61 | 21.32 |
| | | deviation | | |
| | | Minimum value | −7.0 | −15.0 |
| | | Median value | 10.00 | 8.50 |
| | | Maximum | 20.0 | 47.0 |
| | | value | | |
| | 100 mg | Number of | 5 | 5 |
| | | subjects | | |
| | | Average value | 2.40 | 3.40 |
| | | Standard | 21.59 | 30.09 |
| | | deviation | | |
| | | Minimum value | −20.0 | −25.0 |
| | | Median value | 0.00 | −5.00 |
| | | Maximum | 37.0 | 52.0 |
| | | value | | |
| | Placebo | Number of | 5 | 5 |
| | | subjects | | |
| | | Average value | 22.00 | 15.80 |
| | | Standard | 9.33 | 16.92 |
| | | deviation | | |
| | | Minimum value | 10.0 | −10.0 |
| | | Median value | 25.00 | 15.00 |
| | | Maximum | 33.0 | 32.0 |
| | | value | | |
| Amount of change in | 10 mg | Number of | 6 | 6 |
| vitality (VT) | | subjects | | |
| | | Average value | −1.05 | 10.42 |
| | | Standard | 29.97 | 17.07 |
| | | deviation | | |
| | | Minimum value | −50.0 | −6.2 |
| | | Median value | 6.25 | 3.10 |
| | | Maximum | 25.0 | 37.5 |
| | | value | | |
| | 30 mg | Number of | 6 | 6 |
| | | subjects | | |
| | | Average value | −4.18 | 1.05 |
| | | Standard | 15.62 | 17.86 |
| | | deviation | | |
| | | Minimum value | −25.0 | −25.0 |
| | | Median value | −6.25 | 3.15 |
| | | Maximum | 18.7 | 25.0 |
| | | value | | |
| | 100 mg | Number of | 5 | 5 |
| | | subjects | | |
| | | Average value | 7.50 | 8.76 |
| | | Standard | 11.17 | 13.71 |
| | | deviation | | |
| | | Minimum value | −6.2 | 0.0 |
| | | Median value | 6.20 | 0.00 |
| | | Maximum | 18.8 | 31.3 |
| | | value | | |
| | Placebo | Number of | 5 | 5 |
| | | subjects | | |
| | | Average value | 8.76 | 17.52 |
| | | Standard | 17.44 | 22.27 |
| | | deviation | | |
| | | Minimum value | −6.2 | −6.2 |
| | | Median value | 0.00 | 12.50 |
| | | Maximum | 37.5 | 43.8 |
| | | value | | |
| Amount of change in | 10 mg | Number of | 6 | 6 |
| social functioning (SF) | | subjects | | |
| | | Average value | 0.00 | 2.08 |
| | | Standard | 41.08 | 35.72 |
| | | deviation | | |
| | | Minimum value | −50.0 | −50.0 |
| | | Median value | 0.00 | 6.25 |
| | | Maximum | 62.5 | 50.0 |
| | | value | | |
| | 30 mg | Number of | 6 | 6 |
| | | subjects | | |
| | | Average value | 10.42 | 20.83 |
| | | Standard | 39.86 | 34.16 |
| | | deviation | | |
| | | Minimum value | −62.5 | −37.5 |
| | | Median value | 18.75 | 31.25 |
| | | Maximum | 50.0 | 50.0 |
| | | value | | |
| | 100 mg | Number of | 5 | 5 |
| | | subjects | | |
| | | Average value | −22.50 | −10.00 |
| | | Standard | 29.84 | 16.30 |
| | | deviation | | |
| | | Minimum value | −50.0 | −37.5 |
| | | Median value | −37.50 | 0.00 |
| | | Maximum | 25.0 | 0.0 |
| | | value | | |
| | Placebo | Number of | 5 | 5 |
| | | subjects | | |
| | | Average value | 12.50 | 17.50 |
| | | Standard | 21.65 | 37.08 |
| | | deviation | | |
| | | Minimum value | 0.0 | −25.0 |
| | | Median value | 0.00 | 12.50 |
| | | Maximum | 50.0 | 75.0 |
| | | value | | |
| Amount of change in role | 10 mg | Number of | 6 | 6 |
| emotional (RE) | | subjects | | |
| | | Average value | −4.17 | 22,23 |
| | | Standard | 31.97 | 32.36 |
| | | deviation | | |
| | | Minimum value | −41.7 | −25.0 |
| | | Median value | −8.35 | 25.00 |
| | | Maximum | 50.0 | 66.7 |
| | | value | | |
| | 30 mg | Number of | 6 | 6 |
| | | subjects | | |
| | | Average value | −1.38 | 6.93 |
| | | Standard | 28.11 | 50.67 |
| | | deviation | | |
| | | Minimum value | −25.0 | −50.0 |
| | | Median value | −12.50 | 0.00 |
| | | Maximum | 41.7 | 66.7 |
| | | value | | |
| | 100 mg | Number of | 5 | 5 |
| | | subjects | | |
| | | Average value | −16.68 | −10.00 |
| | | Standard | 28.25 | 27.86 |
| | | deviation | | |
| | | Minimum value | −58.3 | −58.3 |
| | | Median value | 0.00 | 0.00 |
| | | Maximum | 8.3 | 8.3 |
| | | value | | |
| | Placebo | Number of | 5 | 5 |
| | | subjects | | |
| | | Average value | 5.00 | −3.30 |
| | | Standard | 28.03 | 32.08 |
| | | deviation | | |
| | | Minimum value | −25.0 | −50.0 |
| | | Median value | 0.00 | 0.00 |
| | | Maximum | 41.7 | 33.4 |
| | | value | | |
| Amount of change in | 10 mg | Number of | 6 | 6 |
| mental health (MH) | | subjects | | |
| | | Average value | 0.00 | 10.83 |
| | | Standard | 30.82 | 13.20 |
| | | deviation | | |
| | | Minimum value | −35.0 | 0.0 |
| | | Median value | −7.50 | 5.00 |
| | | Maximum | 50.0 | 30.0 |
| | | value | | |
| | 30 mg | Number of | 6 | 6 |
| | | subjects | | |
| | | Average value | −2.50 | 15.83 |
| | | Standard | 14.75 | 34.56 |
| | | deviation | | |
| | | Minimum value | −25.0 | −10.0 |
| | | Median value | 2.50 | 5.00 |
| | | Maximum | 15.0 | 80.0 |
| | | value | | |
| | 100 mg | Number of | 5 | 5 |
| | | subjects | | |
| | | Average value | −5.00 | 2.00 |
| | | Standard | 15.41 | 21.97 |
| | | deviation | | |
| | | Minimum value | −25.0 | −25.0 |
| | | Median value | 0.00 | 10.00 |
| | | Maximum | 15.0 | 30.0 |
| | | value | | |
| | Placebo | Number of | 5 | 5 |
| | | subjects | | |
| | | Average value | 9.00 | 17.00 |
| | | Standard | 15.17 | 21.97 |
| | | deviation | | |
| | | Minimum value | −15.0 | −10.0 |
| | | Median value | 15.00 | 25.00 |
| | | Maximum | 25.0 | 45.0 |
| | | value | | |

TABLE 46

| | | | | Analysis of variance table | | | |
|---|---|---|---|---|---|---|---|
| | | | | | Degrees of | | |
| Subscale | Dosing | | Number of | | freedom | F- | p- |
| item | group | | subjects | Factor | (numerator) | value | value |
| Amount of | 10 | mg | 6 | Dosing group | 3 | 0.12 | 0.9501 |
| change in | 30 | mg | 6 | Time of | 1 | 2.49 | 0.1318 |
| physical | | | | measurement | | | |
| functioning | 100 | mg | 6 | Dosing group × | 3 | 0.89 | 0.4647 |
| (PF) | | | | time of | | | |
| | | | | measurement | | | |
| | Placebo | | 6 | | | | |
| | Active | | 18 | Dosing group | 1 | 0.03 | 0.8661 |
| | group* | | | | | | |
| | Placebo | | 6 | Time of | 1 | 4.11 | 0.0562 |
| | | | | measurement | | | |
| | | | | Dosing group × | 1 | 1.67 | 0.2104 |
| | | | | time of | | | |
| | | | | measurement | | | |
| Amount of | 10 | mg | 6 | Dosing group | 3 | 0.86 | 0.4796 |
| change in | 30 | mg | 6 | Time of | 1 | 3.41 | 0.0814 |
| role | | | | measurement | | | |
| physical | 100 | mg | 6 | Dosing group × | 3 | 1.22 | 0.3315 |
| (RP) | | | | time of | | | |
| | | | | measurement | | | |
| | Placebo | | 6 | | | | |
| | Active | | 18 | Dosing group | 1 | 0.12 | 0.7319 |
| | group* | | | | | | |
| | Placebo | | 6 | Time of | 1 | 1.19 | 0.2879 |
| | | | | measurement | | | |
| | | | | Dosing group × | 1 | 0.68 | 0.4199 |
| | | | | time of | | | |
| | | | | measurement | | | |
| | | | | Analysis of variance table | | | |
| | | | | | Degrees of | | |
| Subscale | Dosing | | Number of | | freedom | F- | p- |
| item | group | | subjects | Factor | (numerator) | value | value |
| Amount of | 10 | mg | 6 | Dosing group | 3 | 0.97 | 0.4279 |
| change in | 30 | mg | 6 | Time of | 1 | 11.33 | 0.0034 |
| bodily pain | | | | measurement | | | |
| (BP) | 100 | mg | 6 | Dosing group × | 3 | 0.80 | 0.5103 |
| | | | | time of | | | |
| | | | | measurement | | | |
| | Placebo | | 6 | | | | |
| | Active | | 18 | Dosing group | 1 | 3.07 | 0.0949 |
| | group* | | | | | | |
| | Placebo | | 6 | Time of | 1 | 8.94 | 0.0072 |
| | | | | measurement | | | |
| | | | | Dosing group × | 1 | 0.03 | 0.8615 |
| | | | | time of | | | |
| | | | | measurement | | | |
| Amount of | 10 | mg | 6 | Dosing group | 3 | 0.87 | 0.4755 |
| change in | 30 | mg | 6 | Time of | 1 | 0.11 | 0.7385 |
| general health | | | | measurement | | | |
| (GH) | 100 | mg | 6 | Dosing group × | 3 | 0.34 | 0.7990 |
| | | | | time of | | | |
| | | | | measurement | | | |
| | Placebo | | 6 | | | | |
| | Active | | 18 | Dosing group | 1 | 2.07 | 0.1656 |
| | group* | | | | | | |
| | Placebo | | 6 | Time of | 1 | 0.67 | 0.4229 |
| | | | | measurement | | | |
| | | | | Dosing group × | 1 | 1.06 | 0.3160 |
| | | | | time of | | | |
| | | | | measurement | | | |
| | | | | Analysis of variance table | | | |
| | | | | | Degrees of | | |
| Subscale | Dosing | | Number of | | freedom | F- | p- |
| item | group | | subjects | Factor | (numerator) | value | value |
| Amount of | 10 | mg | 6 | Dosing group | 3 | 0.75 | 0.5345 |
| change in | 30 | mg | 6 | Time of | 1 | 2.74 | 0.1151 |
| vitality | | | | measurement | | | |
| (VT) | 100 | mg | 6 | Dosing group × | 3 | 0.30 | 0.8260 |
| | | | | time of | | | |
| | | | | measurement | | | |
| | Placebo | | 6 | | | | |
| | Active | | 18 | Dosing group | 1 | 1.36 | 0.2568 |
| | group* | | | | | | |
| | Placebo | | 6 | Time of | 1 | 2.61 | 0.1219 |
| | | | | measurement | | | |
| | | | | Dosing group × | 1 | 0.07 | 0.7912 |
| | | | | time of | | | |
| | | | | measurement | | | |
| Amount of | 10 | mg | 6 | Dosing group | 3 | 1.19 | 0.3423 |
| change in | 30 | mg | 6 | Time of | 1 | 2.18 | 0.1571 |
| social | | | | measurement | | | |
| functioning | 100 | mg | 6 | Dosing group × | 3 | 0.23 | 0.8765 |
| (SF) | | | | time of | | | |
| | | | | measurement | | | |

| | | | | | |
|---|---|---|---|---|---|
| Placebo | 6 | | | | |
| Active | 18 | Dosing group | 1 | 0.73 | 0.4040 |
| group* | | | | | |
| Placebo | 6 | Time of | 1 | 1.26 | 0.2744 |

| | | | | measurement | | | |
|---|---|---|---|---|---|---|---|
| | | | | Dosing group × | 1 | 0.07 | 0.7936 |
| | | | | time of | | | |
| | | | | measurement | | | |
| | | | | Analysis of variance table | | | |
| | | | | | Degrees of | | |
| Subscale | Dosing | | Number of | | freedom | F- | p- |
| item | group | | subjects | Factor | (mimerator) | value | value |
| Amount of | 10 | mg | 6 | Dosing group | 3 | 0.54 | 0.6616 |
| change in | 30 | mg | 6 | Time of | 1 | 1.50 | 0.2359 |
| role | | | | measurement | | | |
| emotional | 100 | mg | 6 | Dosing group × | 3 | 1.12 | 0.3666 |
| (RE) | | | | time of | | | |
| | | | | measurement | | | |
| | Placebo | | 6 | | | | |
| | Active | | 18 | Dosing group | 1 | 0.00 | 0.9681 |
| | group* | | | | | | |
| | Placebo | | 6 | Time of | 1 | 0.14 | 0.7118 |
| | | | | measurement | | | |
| | | | | Dosing group × | 1 | 2.03 | 0.1693 |
| | | | | time of | | | |
| | | | | measurement | | | |
| Amount of | 10 | mg | 6 | Dosing group | 3 | 0.60 | 0.6219 |
| change in | 30 | mg | 6 | Time of | 1 | 3.06 | 0.0974 |
| mental | | | | measurement | | | |
| health | 100 | mg | 6 | Dosing group × | 3 | 0.17 | 0.9150 |
| (MH) | | | | time of | | | |
| | | | | measurement | | | |

| | | | | | |
|---|---|---|---|---|---|
| Placebo | 6 | | | | |
| Active | 18 | Dosing group | 1 | 1.18 | 0.2894 |
| group* | | | | | |
| Placebo | 6 | Time of | 1 | 2.00 | 0.1731 |

| | | | |
|---|---|---|---|
| measurement | | | |
| Dosing group × | 1 | 0.09 | 0.7656 |
| time of | | | |
| measurement | | | |

*Combined with 10 mg, 30 mg, and 100 mg dosing groups

### Relationship Between LVEF and LVESVI

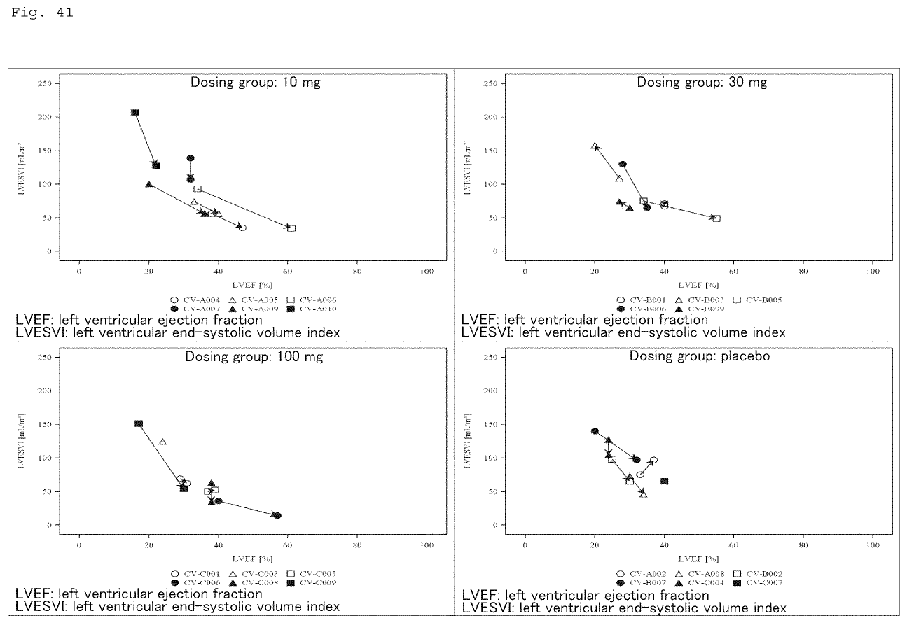

In order to examine the correlation between the improvement of left ventricular remodeling and the improvement of cardiac function, i.e., the correlation between the decrease in LVESVI in cardiac-gated CT and the increase in LVEF in echocardiography, LVEF values were plotted on the x-axis and LVESVI values on the y-axis, and the movement of each subject between two time points of the baseline and 26 weeks after administration was plotted for each dose group, and shown in FIG. 41. In addition, the percentage of cases with a right downward straight line (the coefficient of the slope of the straight line was negative and the LVESVI value at 26 weeks after administration was lower than the baseline value) was calculated for each dose group, and shown in Table 47.

The number of cases with a right downward straight line was 5/6 cases (83.3%), 2/5 cases (40.0%), 3/6 cases (50.0%), and 3/6 cases (50.0%) in the order of the YS-1402−10 mg group, 30 mg group, 100 mg group, and placebo group. There was no dose-dependent increase.

TABLE 47

| | | Number of subjects with negative* |
|---|---|---|
| Dosing group | Number of subjects | slope of straight line |
| 10 mg | 6 | 5 (83.3) |
| 30 mg | 5 | 2 (40.0) |
| 100 mg | 6 | 3 (50.0) |
| Placebo | 6 | 3 (50.0) |

( ): %
*When LVEF (left ventricular ejection fraction) is plotted on the x-axis, and LVESVI (left ventricular end-systolic volume index) on the y-axis, the slope between IC/S and 26 weeks after administration is negative, and the LVESVI value at 26 weeks after administration is lower than the value at IC/S.

Conclusion of the pharmacokinetic analysis of ONO-1301 when YS-1402 was attached to the left ventricle during coronary artery bypass surgery for ischemic cardiomyopathy

The pharmacokinetics of active ONO-1301 in blood when YS-1402 was attached to the left ventricle during coronary artery bypass surgery for ischemic cardiomyopathy were checked.

Regarding the summary statistics of the pharmacokinetic parameters of ONO-1301 in blood, in the order of the YS-1402−10 mg group, 30 mg group, and 100 mg group, Cmax (mean±standard deviation, hereinafter the same) was 2.0788±1.1579, 4.2967±1.5310, 8.8383±2.1971 ng/ml, and AUC0-t was 1059.9076±522.3988, 2640.5036±730.4192, 5572.9516±1190.7685 ng·h/ml. Cmax and AUC0-t increased with dose. On the other hand, when Cmax and AUC0-t in the YS-1402−10 mg group were each set to 1, Cmax and AUC0-t in the 30 mg group were 2.07 and 2.49 times, respectively, and similarly 4.25 and 5.26 times in the 100 mg group, which were less than the common ratio. MRT0-t was almost constant.

The blood concentration of ONO-1301 increased after administration and reached a plateau 7 to 14 days after administration in the YS-1402−10 mg group and 30 mg group. The blood concentration in the 100 mg group continually changed from about 4 ng/ml to 9 ng/ml from 24 hours after administration to 28 days, and then decreased rapidly. In all groups, drug concentrations in the blood almost disappeared 8 weeks after administration. The maximum Cmax for ONO-1301 in the YS-1402−100 mg group, which was the maximum dose group, was 11.900 ng/ml, which did not exceed the no-observed-effect level 15.61 ng/ml and the maximum no-observed-adverse-effect level 23.69 ng/ml, satisfying the secondary hypothesis that Cmax was 23.7 ng/ml or less.

Conclusion of the analysis of changes in indicators related to the improvement of cardiac function when YS-1402 was attached to the left ventricle during coronary artery bypass surgery for ischemic cardiomyopathy

Changes in indicators related to the improvement of cardiac function when YS-1402 was attached to the left ventricle during coronary artery bypass surgery for ischemic cardiomyopathy were searched. For the site of attachment, the area of reduced myocardial blood flow was identified in advance by preoperative ammonia PET. Lesion sites such as highly fibrotic regions and poor contraction regions in complex lesions and multivessel lesions where complete blood flow recovery to cardiac ischemia was difficult were also identified visually and tactilely in the coronary artery bypass surgery, and sheets were attached to the left ventricle site, including the peripheral part, where graft running was not affected.

For each evaluation item, as a result of analysis of variance of repeated measurements with factors of dose group, time of measurement, and dose group×time of measurement, variations in dose group and dose group×time of measurement were not significant. The results were similar for the two groups: the active groups combined with three doses of YS-1402 and the placebo group. On the other hand, variation in time of measurement was significant for various evaluation items, suggesting that coronary artery bypass surgery had a significant effect.

Regarding the amount of change in LVEF in echocardiography, the placebo group showed a slight increase over time. Compared to the placebo group, all the YS-1402 dosing groups showed improvement at 26 weeks after administration; however, there was no dose-related improvement. At 26 weeks after administration, the 10 mg dosing group showed improvement with an amount of change of 5.8% compared to the placebo group. A positive correlation was observed between the rate of change in total myocardial blood flow and the amount of change in LVEF from the baseline at 26 weeks after administration of the investigational drug; however, the correlation was not significant (p-value: 0.340). One case in the YS-1402 30 mg group developed a severe adverse event (congestive heart failure) due to poor medication compliance 26 weeks after administration and 1 week before the test, which significantly decreased LVEF and thus also affected the average value of LVEF.

The rate of change in CT in cardiac-gated CT decreased in the placebo group at 2 weeks after administration, but increased at 26 weeks more than before administration. The YS-1402 groups generally showed an increase over time, surpassing the placebo group at 26 weeks, and a dose-related increase was observed. Compared to the placebo group, the 100 mg dosing group showed improvement with a rate of change of 10.16%. A positive correlation was observed between the rate of change in total myocardial blood flow and the rate of change in CT from the baseline at 26 weeks after administration of the investigational drug; however, the correlation was not significant (p-value: 0.102).

Regarding changes in left ventricular remodeling, the rate of change in LVESVI in cardiac-gated CT decreased over time in the placebo group. The YS-1402 groups also showed a decrease over time. Particularly at 2 weeks, the degree of decrease in the YS-1402 groups was greater than in the placebo group; however, the decrease was not dose-dependent. Considering that a reduction of 10% or more in the rate of change in LVESVI is reportedly used as a criterion for cardiac resynchronization therapy responder, and in consideration of measurement errors, fluctuations within 10% were defined as “constant.” Then, aggregation was performed for each subject. As a result, decrease/constant/increase were 1/5/0 and 4/0/1 in the placebo group, in the order of 2 weeks and 26 weeks after administration. Similarly, these were 3/2/0 and 6/0/0 in the YS-1402−10 mg group, 3/2/0 and 3/1/2 in the 30 mg group, and 4/2/0 and 4/I/O in the 100 mg group. The placebo group generally showed improvement over time. Compared to the placebo group, the YS-1402 groups showed a better degree of improvement at 2 weeks after administration, and was dose-dependent. At week 26, there was an increase in subjects in the YS-1402 30 mg group and placebo group, while the degree of improvement in the 10 mg group and 100 mg group was good, but was not dose-dependent. The rate of change in LVEDVI in cardiac-gated CT decreased over time in the placebo group. Similarly, the YS-1402 groups generally showed a decrease over time; however, it was not dose-dependent. The rate of change in LVDs and LVDd in echocardiography decreased over time in the placebo group. The YS-1402 groups showed a greater decrease than the placebo group at the second week of administration; however, it was not dose-dependent. The decrease was not temporal or dose-dependent at 6 weeks and 26 weeks after administration. The amount of change in CTR in chest X-ray reached its maximum on the first day after administration, and then decreased over time in the placebo group. Similarly, the YS-1402 groups showed an approximate maximum value on the first day after administration, and then showed a decrease over time. The maximum values and midway values were not dose-dependent.

Regarding changes in symptoms of heart failure, both the placebo group and the YS-1402 groups showed a similar degree of improvement over time for changes in the NYHA classification. Particularly in the 100 mg dosing group, all cases were improved to Level I at 26 weeks after administration. Regarding the rate of change in 6-minute walking distance, both the placebo group and the YS-1402 groups generally showed an increase in the walking distance over time, and at 6 weeks after administration, the YS-1402 groups showed a greater degree of increase than the placebo group. On the other hand, the 6-minute walking distance at 26 weeks after administration was similar to the placebo group in the 10 mg group, and exceeded the placebo group in the 100 mg group, showing an increase with a rate of change of 6.49% compared to the placebo group. The 30 mg group was lower than the placebo group. The reason for this was thought to be that one case in the 30 mg group developed a severe adverse event (congestive heart failure) due to poor medication compliance 26 weeks after administration and 1 week before the test, resulting in a significant decrease in distance from before administration. Therefore, for reference, the 26-week data of this case was excluded; however, there was no dose-related increase.

Regarding changes in myocardial blood flow by ammonia PET, in the placebo group, the LAD resting myocardial blood flow, LCX resting myocardial blood flow, and total myocardial blood flow at 26 weeks after administration slightly increased compared to the baseline. At 26 weeks after administration, a dose-related increase in blood flow was observed in the LAD resting myocardial blood flow, RCA resting myocardial blood flow, and total myocardial blood flow in the YS-1402−10 mg group, 30 mg group, and 100 mg group. In the 100 mg group, the LAD resting myocardial blood flow, RCA resting myocardial blood flow, and total myocardial blood flow at 26 weeks after administration increased compared to the placebo group as shown below: the rate of change in LAD resting myocardial blood flow was 21.18%, the rate of change in RCA resting myocardial blood flow was 17.04%, and the rate of change in total myocardial blood flow was 14.77%. On the other hand, the LCX resting myocardial blood flow did not show a dose-dependent increase. A positive correlation was observed between AUC0-t and the rate of change in total myocardial blood flow from the baseline at 26 weeks after administration of the investigational drug; however, the correlation was not significant (p-value: 0.160).

No definite tendency was observed in blood BNP at 26 weeks after administration.

Regarding SF-36, which was set for QOL assessment, transition was different for each subscale, and no definite tendency was observed.

There was no dose-response relationship between LVEF and LVESVI.

As a result of investigating the relationship over time between myocardial blood flow, cardiac function (CT, LVEF), and 6-minute walking distance in the placebo group and YS-1402 dosing groups, the myocardial blood flow increased at 6 weeks after administration. At 26 weeks, even if it was similar or lower than that at 6 weeks, the cardiac function (CT, LVEF) was higher at 26 weeks than at 6 weeks (2 weeks for CT), and in some cases, the 6-minute walking distance increased. This suggested that the cardiac function and the symptoms of heart failure are improved with the increase in myocardial blood flow, and that the improvement in the cardiac function and the symptoms of heart failure continues even after the increase in myocardial blood flow has ceased. For example, in the YS-1402−100 mg group, the myocardial blood flow at 26 weeks after administration generally increased compared to 6 weeks, and the improvement in cardiac function (CT, LVEF) and the increase in 6-minute walking distance were recognized, suggesting the need for follow-up after 26 weeks.

### Evaluation of Safety

### 1) Adverse Events

Table 48 shows the frequency of occurrence of adverse events and side effects.

Adverse events were observed in all 6 cases in any dosing group. Of these, those that were determined to be side effects were 2 cases (33.3%) in the YS-1402−10 mg group, 1 case (16.7%) in the 30 mg group, and 1 case (16.7%) in the placebo group. No cases of side effects were observed in the 100 mg group.

Serious adverse events were observed in 2 cases (33.3%) in the YS-1402−10 mg group and 30 mg group, 3 cases (50.0%) in the 100 mg group, and 2 cases (33.3%) in the placebo group. Of these, those that were determined to be serious side effects were pneumonia in 1 case (16.7%) in the YS-1402−10 mg group and lung abscess in 1 case (16.7%) in the placebo group.

There were no adverse events leading to discontinuation or deaths.

TABLE 48

| | Dosing | Number of | Number of | |
|---|---|---|---|---|
| Endpoints | group | subjects | occurrences | Incidence |
| Adverse events | 10 mg | 6 | 6 | 100.0 |
| | 30 mg | 6 | 6 | 100.0 |
| | 100 mg | 6 | 6 | 100.0 |
| | Placebo | 6 | 6 | 100.0 |
| Serious adverse | 10 mg | 6 | 2 | 33.3 |
| events | 30 mg | 6 | 2 | 33.3 |
| | 100 mg | 6 | 3 | 50.0 |
| | Placebo | 6 | 2 | 33.3 |
| Side effects | 10 mg | 6 | 2 | 33.3 |
| | 30 mg | 6 | 1 | 16.7 |
| | 100 mg | 6 | 0 | 0.0 |
| | Placebo | 6 | 1 | 16.7 |
| Serious side | 10 mg | 6 | 1 | 16.7 |
| effects | 30 mg | 6 | 0 | 0.0 |
| | 100 mg | 6 | 0 | 0.0 |
| | Placebo | 6 | 1 | 16.7 |

Tables 49 and 50 show the frequency of occurrence of adverse events and side effects by SOC/PT, Tables 51 and 52 show the frequency of occurrence by degree, and Tables 53 and 54 show the frequency of occurrence by period of occurrence. Further, Table 55 shows the frequency of occurrence of adverse events by causal relationship, and Table 56 shows the frequency of occurrence by outcome.

By PT, abnormal clinical test values were frequently observed due to coronary artery bypass surgery; thus, the adverse events were classified into those of clinical test and those other than clinical test. That is, examination was performed on adverse events other than clinical test that occurred in at least 2 cases (33.3%) in any of the dosing groups by PT, and adverse events of clinical test that occurred in at least 5 cases (83.3%) in any of the dosing groups in the same way.

The adverse events other than clinical test were atrial fibrillation, tachycardia, diarrhea, edema, fever, dehydration, restlessness, sleep disturbance, and pleural effusion. The adverse events of clinical test were increased alanine aminotransferase, increased aspartate aminotransferase, decreased blood albumin, increased blood creatine phosphokinase, increased blood lactate dehydrogenase, increased C-reactive protein, decreased hematocrit, decreased hemoglobin, decreased lymphocyte count, increased neutrophil count, decreased platelet count, decreased erythrocyte count, increased platelet count, and increased brain natriuretic peptide. Of these, pleural effusion was suspected to be related to the dose of YS-1402. Other than the above, there were no adverse events of clinical test with a suspected relationship, including four cases or less of adverse events in any of the dosing groups.

Pleural effusion was observed in 4 cases (66.7%) in the YS-1402−10 mg group, 6 cases (100.0%) in each of the 30 mg group and 100 mg group, and 2 cases (33.3%) in the placebo group. The severity of the disease was mild in 4 cases (66.7%) in the YS-1402−10 mg group, mild in 5 cases (83.3%) and moderate in 1 case (16.7%) in the 30 mg group, mild in 1 case (16.7%) and moderate in 5 cases (83.31) in the 100 mg group, and mild in 2 cases (33.3%) in the placebo group. The time of onset was within 1 week after administration, and the outcome was recovery in all cases. The cause of pleural effusion was attributed to coronary artery bypass surgery and heart failure, and it was not considered to be a side effect. Pleural effusion could be treated by administration of diuretics or puncture, and was determined not to be a serious adverse event.

Among the adverse events, those that were determined to be side effects were 2 cases (33.3) in the YS-1402−10 mg group, 1 case (16.73) in the 30 mg group, and 1 case (16.7%) in the placebo group.

The aggregation by PT showed 1 case of pneumonia (16.7%) and 1 case of increased blood triglyceride and increased blood uric acid (16.7%) in the YS-1402−10 mg group, 1 case of increased alanine aminotransferase and increased aspartate aminotransferase (16.7%) in the 30 mg group, and 1 case of lung abscess (16.7%) in the placebo group. Of these, those determined to be serious were pneumonia in the YS-1402−10 mg group and lung abscess in the placebo group, and the severity was high in pneumonia and moderate in lung abscess. All four items in the clinical test were non-serious and mild.

Lengthy table referenced here
US20240197753A1-20240620-T00001
Please refer to the end of the specification for access instructions.

Lengthy table referenced here
US20240197753A1-20240620-T00002
Please refer to the end of the specification for access instructions.

Lengthy table referenced here
US20240197753A1-20240620-T00003
Please refer to the end of the specification for access instructions.

Lengthy table referenced here
US20240197753A1-20240620-T00004
Please refer to the end of the specification for access instructions.

Lengthy table referenced here
US20240197753A1-20240620-T00005
Please refer to the end of the specification for access instructions.

Lengthy table referenced here
US20240197753A1-20240620-T00006
Please refer to the end of the specification for access instructions.

Lengthy table referenced here
US20240197753A1-20240620-T00007
Please refer to the end of the specification for access instructions.

Lengthy table referenced here
US20240197753A1-20240620-T00008
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240197753A1). An electronic copy of the table will also be available from the USPTO upon request and payment of the fee set forth in 37 CFR 1.19(b)(3).

1. A pharmaceutical composition for improving cardiac function, comprising:
(A) a release formulation comprising at least poly(lactic-co-glycolic acid) (PLGA) and a prostaglandin 12 receptor agonist, the PLGA having an average molecular weight of 10000 to 30000; and
(B) a release formulation comprising at least poly(lactic-co-glycolic acid) (PLGA) and a prostaglandin 12 receptor agonist, the PLGA having an average molecular weight of 40000 to 60000.

2. The pharmaceutical composition for improving cardiac function according to claim 1, wherein the ratio of the release formulation (B) to the release formulation (A) (A:B) is 1:1 to 100:1 or 1:1 to 1:100.

3. The pharmaceutical composition for improving cardiac function according claim 1, wherein the release formulation (A) comprises 0.5 to 50 mg of PGI2 receptor agonist in one vial, and/or the release formulation (B) comprises 0.5 to 50 mg of PGI2 receptor agonist in one vial.

4. The pharmaceutical composition according to claim 1, comprising a patch liquid.

5. The pharmaceutical composition according to claim 4, wherein the patch liquid is a 5 w/v % mannitol aqueous solution comprising 0.2 w/v % of polysorbate.

6. The pharmaceutical composition according to claim 1, comprising a gelatin patch.

7. The pharmaceutical composition according to claim 6, wherein the gelatin patch is a porous sterile formulation comprising 10 g of gelatin per 1000 cm³.

8. The pharmaceutical composition according to claim 1, comprising a plasma fraction formulation.

9. The pharmaceutical composition according to claim 8, wherein the plasma fraction formulation comprises a fibrinogen powder, an aprotinin solution, a thrombin powder, and a calcium chloride solution.

10. The pharmaceutical composition according to claim 1, comprising, as the prostaglandin 12 receptor agonist, at least a compound of the following formula (I) or a salt thereof: 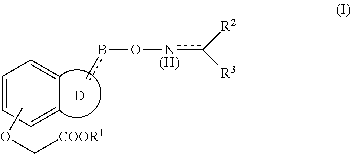
wherein 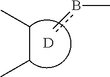
is 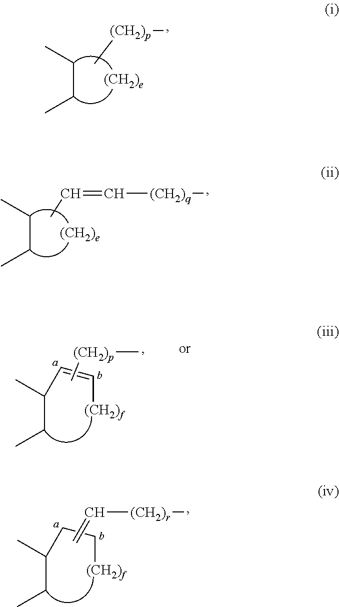
wherein
R¹ represents a hydrogen atom or a C1−4 alkyl group,
R² represents (i) a hydrogen atom, (ii) a C1-8 alkyl group, (iii) a phenyl group or a C4−7 cycloalkyl group, (iv) a 4- to 7-membered monocyclic ring containing one nitrogen atom, (v) a C1−4 alkyl group substituted with a benzene ring or a C4−7 cycloalkyl group, or (vi) a C1−4 alkyl group substituted with a 4- to 7-membered monocyclic ring containing one nitrogen atom,
R³ represents (i) a C1-8 alkyl group, (ii) a phenyl group or a C4−7 cycloalkyl group, (iii) a 4- to 7-membered monocyclic ring containing one nitrogen atom, (iv) a C1−4 alkyl group substituted with a benzene ring or a C4−7 cycloalkyl group, or (v) a C1−4 alkyl group substituted with a 4- to 7-membered monocyclic ring containing one nitrogen atom,
e represents an integer of 3 to 5,
f represents an integer of 1 to 3,
p represents an integer of 1 to 4,
q represents 1 or 2, and
r represents an integer of 1 to 3;
provided that when 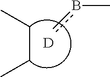
is a group represented by (iii) or (iv) above,
(CH₂)p- and ═CH—(CH₂)s- bind to the position of a or b on the ring, and
ring structures in R² and R³ are optionally substituted with 1 to 3 C1-4 alkyl groups, C1-4 alkoxy groups, halogen atoms, nitro groups, or trihalomethyl groups.

11. The pharmaceutical composition according to claim 1, comprising, as the prostaglandin 12 receptor agonist, at least the following compound (A) or a salt thereof:
(A) ({5-[2−(([(1E)-phenyl(pyridin-3-yl)methylene]amino)oxy)ethyl]-7,8-dihydronaphthalen-1-yl}oxy)acetic acid (ONO-1301) represented by the following formula (II):

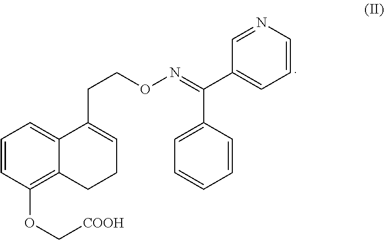 [SMILES: O=C(O)COc1cccc2c1CCC=C2CCO/N=C(\c1ccccc1)c1cccnc1]

12. The pharmaceutical composition according to claim 1, wherein the pharmaceutical composition is a sheet patch.

13. The pharmaceutical composition according to claim 1, wherein the pharmaceutical composition is administered to a patient with ischemic cardiomyopathy who undergoes coronary artery bypass surgery.

14. The pharmaceutical composition according to claim 1, wherein the prostaglandin 12 receptor agonist is released over 4 weeks after administration.

15. The pharmaceutical composition according to claim 1, which is a sustained-release formulation of microspheres (MS).

16. The pharmaceutical composition according to claim 15, wherein the sustained-release formulation has an average particle size of 3 to 300 μm.
